(19) United States
(12) Patent Application Publication
(10) Pub. No.: US 2019/0201410 A1
(43) Pub. Date: Jul. 4, 2019

(54) TRACE AMINE ASSOCIATED RECEPTOR 1 AGONISTS AND PARTIAL AGONISTS FOR PAIN TREATMENT

(71) Applicant: Purdue Pharma L.P., Stamford, CT (US)

(72) Inventors: Michele HUMMEL, Marlton, NJ (US); Donald J. KYLE, Yardley, PA (US); Laykea TAFESSE, Robbinsville, NJ (US); Garth WHITESIDE, Yardley, PA (US)

(73) Assignee: Purdue Pharma L.P., Stamford, CT (US)

(21) Appl. No.: 16/306,305

(22) PCT Filed: Jun. 2, 2017
(86) PCT No.: PCT/US17/35776

§ 371 Date: Nov. 30, 2018

Related U.S. Application Data
(60) Provisional application No. 62/344,833, filed on Jun. 2, 2016.
(60) Provisional application No. 62/440,747, filed on Dec. 30, 2016.

(51) Int. Cl. A61K 31/5386; A61K 31/421; A61K 31/455; A61K 31/4174; A61K 31/402; A61K 31/4439; A61K 31/5377; A61P 25/02

(52) U.S. Cl. CPC A61K 31/5386; A61K 31/421; A61K 31/455; A61P 25/02; A61K 31/402; A61K 31/4439; A61K 31/5377; A61K 31/4174

(57) ABSTRACT

This application relates to using trace amine associated receptor 1 (TAAR1) agonists and/or partial agonists for treating or preventing pain, especially neuropathic pain.

### FIELD OF INVENTION

The application relates to methods of treating or preventing pain (e.g., chronic pain) by administering a trace amine associated receptor 1 (“TAAR1”) agonist or partial agonist to a subject in need of such treatment or prevention.

### BACKGROUND OF THE INVENTION

Pain is the most common symptom for which patients seek medical advice and treatment. While acute pain is usually self-limited, chronic pain can persist for 3 months or longer and lead to significant changes in a patient's personality, lifestyle, functional ability and overall quality of life (K. M. Foley, Pain, in Cecil Textbook of Medicine 100-107, J. C. Bennett and F. Plum eds., 20th ed. 1996).

Chronic pain can be classified as either nociceptive or neuropathic. Nociceptive pain includes tissue injury-induced pain and inflammatory pain such as that associated with arthritis. Neuropathic pain is caused by damage to the peripheral or central nervous system and is maintained by aberrant somatosensory processing.

Neuropathic pain is a common variety of chronic pain. It can be defined as pain that results from an abnormal functioning of the peripheral and/or central nervous system. A critical component of this abnormal functioning is an exaggerated response of pain-related nerve cells either in the periphery or in the central nervous system. An example is the pain from causalgia wherein even a light touch to the skin is felt as an excruciating burning pain. Another example is allodynia, wherein stimuli which do not normally provoke pain can trigger a pain response.

Pain has been traditionally managed by administering non-opioid analgesics, such as acetylsalicylic acid, choline magnesium trisalicylate, acetaminophen, ibuprofen, fenoprofen, diflusinal, and naproxen; or opioid analgesics, including morphine, hydromorphone, methadone, levorphanol, fentanyl, oxycodone, and oxymorphone. In addition to the above-listed treatments, neuropathic pain, which can be difficult to treat, has also been treated with anti-epileptics (e.g. gabapentin, carbamazepine, valproic acid, topiramate, phenytoin); NMDA antagonists (e.g. ketamine, dextromethorphan); topical lidocaine (for post-herpetic neuralgia); and tricyclic antidepressants (e.g. fluoxetine, sertraline and amitriptyline).

The long term administration of narcotic analgesics to patients suffering from various types of chronic pain is subject to a number of serious drawbacks including the development of opiate tolerance and/or dependence, severe constipation, and so forth. Therefore, there is a need to develop novel methods for treating pain, especially chronic pain, such as a neuropathic pain.

TAAR1 is a 7-transmembrane domain G-protein coupled receptor (Gas) that responds to various trace amines (“TAs”) (Lindemann et al., “Trace amine-associated receptors form structurally and functionally distinct subfamilies of novel G protein-coupled receptors,” Genomics 85 (3): 372-85 (2005)). TAs include β-phenylethylamine, p-tyramine, tryptamine, octopamine, and synephrine. TAs are activated by thyroid hormone derivative, COMT (Catechol-O-Methyltransferase) products, and amphetamine. TAAR1 has low affinity for classic monoamines. TAAR1 signals through the cAMP/PKA (Protein Kinase A)/CREB (cAMP Responsive Element Binding Protein) and the PKC (Protein Kinase C)/Ca⁺⁺/NFAT (Nuclear Factor of Activated T-cells) pathways.

TAAR1 is expressed in brain, spinal cord, and peripheral tissues in rodents and monkeys (see Xie et al., “Trace Amine-Associated Receptor 1 as a Monoaminergic Modulator in Brain,” Biochem. Pharmacol. 78(9): 1095-1104 (2009)). It has been reported that human TAAR1 mRNA was detected by quantitative reverse transcription (RT)-PCR in low levels in discrete regions within the central nervous system (CNS) and in several peripheral tissues. Moderate levels were expressed in stomach, low levels expressed in amygdala, kidney, lung, and small intestine, whereas trace amounts were expressed in cerebellum, dorsal root ganglia, hippocampus, hypothalamus, liver, medulla, pancreas, pituitary, pontine reticular formation, prostate, skeletal muscle, and spleen (see Borowsky et al., “Trace amines: Identification of a family of mammalian G protein-coupled receptors,” Proc. Natl. Acad. Sci. U.S.A 98(16): 8966-8971 (2001)).

In vitro, TAAR1 activation drives the PKA and PKC cellular signaling cascades that result in inhibition of monoamine uptake and transporter reversal (efflux) in DAT (Dopamine Transporter)/TAAR1, NET (Norepinephrine Transporter)/TAAR1, and SERT (Serotonin Transporter)/TAAR1 co-transfected cells, as well as in mouse and primate striatal (DAT, SERT) and thalamic (NET) synaptosomes ex vivo.

In vivo, TAAR1 selective activation prevents both hyperdopaminergic- and hypoglutamatergic-induced hyperlocomotion in rodents, suggesting anxiolytic- and antipsychotic-like effects (see Revel et al., “TAAR1 activation modulates monoaminergic neurotransmission, preventing hyperdopaminergic and hypoglutamatergic activity,” Proc. Natl. Acad. Sci. U.S.A 108(20): 8485-8490 (2011)).

TAAR1 has been implicated as playing a role in schizophrenia, depression, addiction and Parkinson's disease. The present inventors have discovered that TAAR1 agonists and partial agonists are useful in the treatment of pain, especially neuropathic pain.

### BRIEF SUMMARY OF THE INVENTION

In one aspect, the present disclosure provides a method of treating pain in a subject, comprising administering to a subject in need of such treatment a therapeutically effective amount of a TAAR1 agonist or partial agonist, or a mixture thereof. In one embodiment, the pain to be treated is a neuropathic pain.

In another aspect, the present disclosure provides a method of treating pain in a subject, comprising administering to a subject in need of such treatment a therapeutically effective amount of a compound of Formula (I):

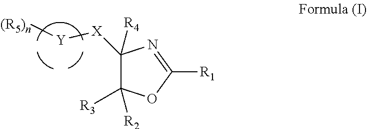

or a pharmaceutically acceptable salt, a racemic mixture, an enantiomer and/or optical isomer (stereoisomer), a tautomer, a prodrug, or a solvate thereof, wherein:

R₁ is NH₂, NH(lower alkyl), or

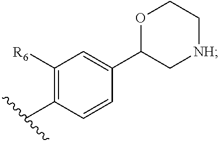

R₂ and R₃ are independently hydrogen or lower alkyl optionally substituted by halogen;

R₄ is hydrogen, phenyl, or lower alkyl;

R₅, each independently, is hydrogen, deuterium, tritium, cyano, halogen, lower alkyl optionally substituted by halogen, lower alkoxy optionally substituted by halogen, phenyl optionally substituted by halogen, phenyloxy, benzyl, benzyloxy, —C(O)O-lower alkyl, —NHC(O)-aryl wherein aryl is optionally substituted by lower alkyl or halogen, —O—(CH₂)ₒ—O-lower alkyl, —NH— cycloalkyl, cycloalkyl, piperidin-1-yl, or tetrahydropyran-4-yloxy, wherein the optional substituents for each R₅ are the same or different;

R₆ is hydrogen or halogen;

X is a bond, —(CHR)ₘ—, —O(CHR)ₘ—, —NRCHR′—, —CHROCHR′—, —SCHR—, —S(O)₂CH₂—, —CH₂SCH₂—, —CH₂N(R)CHR′—, -cycloalkyl-(CHR)ₘ—, or —SiRR′—CH₂—;

R and R′ are each independently hydrogen, lower alkyl optionally substituted by halogen, or benzyl optionally substituted by alkoxy or halogen, and when m>1, each R is the same or different;

Y is aryl, heteroaryl, cycloalkyl, or heterocycloalkyl;

m is 1, 2, 3, or 4;

n is 0, 1, 2, or 3; and

o is 2 or 3;

and

wherein the compound is a TAAR1 agonist or partial agonist.

In one embodiment, the compound of Formula (I) is 4-(3,4-dichlorophenyl)-4,5-dihydrooxazol-2-amine (compound 3), 4-((ethyl(phenyl)amino)methyl)-4,5-dihydrooxazol-2-amine (compound 2), 4-(3-fluoro-2-methylphenyl)-4,5-dihydrooxazol-2-amine, or 4-(2-phenylbutyl)-4,5-dihy drooxazol-2-amine.

In one embodiment, the pain to be treated is a neuropathic pain.

In another aspect, the present disclosure provides a method of treating pain in a subject, comprising administering to a subject in need of such treatment a therapeutically effective amount of a compound of Formula (IIa) or Formula (IIb):

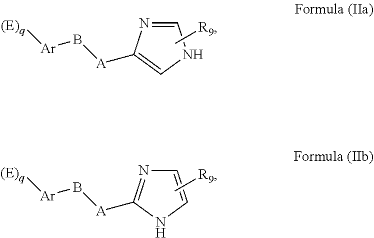

or a pharmaceutically acceptable salt, a racemic mixture, an enantiomer and/or optical isomer (stereoisomer), a tautomer, a prodrug, or a solvate thereof, wherein: —A—B— is —CH(R₈)—, —N(R₇)—CH(R₈)—, —CH(R)—N(R₇)—, —NH—NH—, —O—CH(R₈)—, —CH(R₈)—O—, —S—CH(R₈)—, —CH(R₈)—S—, or —CH(R₈)—CH(R₈)—;

R₇ and R₈ are each independently hydrogen, lower alkyl, lower alkenyl, cycloalkyl, lower alkyl substituted by hydroxy or halogen, —(CH₂)_{d}—S-lower alkyl,

—(CH₂)_{d}—O-lower alkyl, —(CH₂)_{d}—NHC(O)O-lower alkyl, —(CH₂)_{d}-aryl, or —(CH₂)_{d}-heteroaryl;

R₉ is hydrogen, halogen, lower alkyl, or amino;

Ar is phenyl, naphthyl, benzofuranyl, benzo[1,3]dioxolyl, pyrimidin-2-yl, pyrimidin-4-yl, or pyridin-3-yl,

E is hydrogen, halogen, lower alkyl optionally substituted by one or more halogens, lower alkoxy optionally substituted by one or more halogens, —(CH₂)ₚ-aryl, —(CH₂)ₚ-heteroaryl, —O—(CH₂)ₚ-aryl, —O—(CH₂)ₚ-heteroaryl, —(CH₂)ᵣ-phenyl optionally substituted by lower alkoxy, —(CH₂)ᵣ—C(O)-phenyl optionally substituted by lower alkoxy, —(CH₂)ᵣ—O-phenyl optionally substituted by lower alkoxy, cycloalkyl, morpholinyl, NO₂, amino, hydroxy, —CH(OH)-phenyl, or —NHC(O)aryl;

p is 0 or 1;

q is 0, 1, 2, 3, or 4;

r is 0, 1, 2, 3; and

d is 0, 1, 2 or 3;

and

wherein the compound is a TAAR1 agonist or partial agonist.

In another aspect, the present disclosure provides a method of treating pain in a subject, comprising administering to a subject in need of such treatment a therapeutically effective amount of a compound of Formula (III):

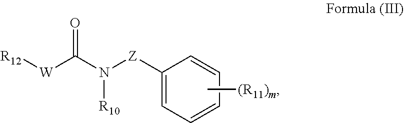

or a pharmaceutically acceptable salt, a racemic mixture, an enantiomer and/or optical isomer (stereoisomer), a tautomer, a prodrug, or a solvate thereof,

wherein:

R₁₀ is hydrogen or lower alkyl;

m is 1 or 2;

R₁₁ independently is —(CH₂)ₖ—(O)ⱼheterocycloalkyl, or —C(O)— heterocycloalkyl, wherein the heterocycloalkyl group is optionally substituted by lower alkyl, hydroxy, halogen, or —(CH₂)_{b}-aryl; or two R₁₁, together with the ring atoms they are attached to, forms heterocycloalkyl optionally substituted by amino;

k is 0, 1, or 2;

j is 0 or 1;

b is 0, 1, or 2;

R₁₂ is (i) lower alkyl, optionally substituted by one or more (same or different) halogens, or cycloalkyl optionally substituted by lower alkoxy or halogen; orindan-2-yl; or

(ii) heterocycloalkyl, optionally substituted by heteroaryl; or

(iii) aryl or heteroaryl, wherein the aromatic rings in aryl and heteroaryl are optionally substituted by one or two substituents independently selected from the group of lower alkyl, halogen, heteroaryl, hydroxy, CF₃, OCF₃, OCH₂CF₃, OCH₂-cycloalkyl, OCH₂C(CH₂OH)(CH₂Cl)(CH₃), S-lower alkyl, lower alkoxy, CH₂-lower alkoxy, lower alkynyl, cyano, —C(O)-phenyl, —O-phenyl, —O—CH₂-phenyl, phenyl, and —CH₂-phenyl, and wherein the phenyl rings are optionally substituted by halogen, —C(O)-lower alkyl, —C(O)OH, or —C(O)O-lower alkyl, or the aromatic rings are optionally substituted by heterocycloalkyl, OCH₂-oxetan-3-yl, or O-tetrahydropyran-4-yl, optionally substituted by lower alkyl;

W is a bond, —N(R₁₃)—, —CH₂NH—, —CH(R₁₄)—, —(CHR₁₄)ᵥ—O—, —O—(CHR₁₄)ᵥ, or —(CH₂)₂—;

Z is a bond or —CH₂—;

R₁₃ is hydrogen or lower alkyl;

R₁₄ is hydrogen, lower alkyl, optionally substituted by one or more (same or different) halogens, or lower alkoxy; and

v is 0, 1, 2, or 3;

and

wherein the compound is a TAAR1 agonist or partial agonist.

In another aspect, the present disclosure provides a method of treating pain in a subject, comprising administering to a subject in need of such treatment a therapeutically effective amount of a compound of Formula (IV):

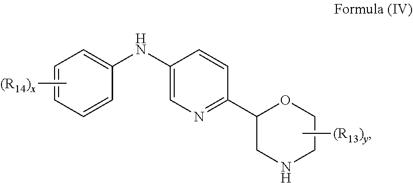

or a pharmaceutically acceptable salt, a racemic mixture, an enantiomer and/or optical isomer (stereoisomer), a tautomer, a prodrug, or a solvate thereof,

wherein:

R₁₃ is independently hydrogen, halogen, alkyl, alkoxy, haloalkyl, haloalkoxy, hydroxyl, cyano, amino, cycloalkyl, heterocycloalkyl, aryl, or heteroaryl;

R₁₄ is independently hydrogen, halogen, alkyl, alkoxy, haloalkyl, haloalkoxy, hydroxyl, cyano, amino, cycloalkyl, heterocycloalkyl, aryl, or heteroaryl;

x is 0, 1, or 2; and

y is 0, 1, or 2;

and

wherein the compound is a TAAR1 agonist or partial agonist.

It is to be understood that in the above “A—B” definition, either end of the group can be directly attached to the imidazole ring.

In one embodiment, the compound of Formulae (IIa) or (IIb) is N-((1H-imidazol-4-yl)methyl)-4-chloro-N-isopropylaniline (compound 1), or 2-(2,6-diethylbenzyl)-1H-imidazole.

In one embodiment, the pain to be treated is a neuropathic pain.

Additional embodiments and advantages of the disclosure will be set forth, in part, in the description that follows, and will flow from the description, or can be learned by practice of the disclosure. The embodiments and advantages of the disclosure will be realized and attained by means of the elements and combinations particularly pointed out in the appended claims.

It is to be understood that both the foregoing summary and the following detailed description are exemplary and explanatory only, and are not restrictive of the invention as claimed.

### BRIEF DESCRIPTION OF THE DRAWINGS/FIGURES

FIG. 1 depicts the effect of a TAAR1 partial agonist (compound 1) on SNL-induced mechanical hyperalgesia in rats following oral (“PO”) dosing.

FIG. 2 depicts the effect of a TAAR1 partial agonist (compound 1) on SNL-induced mechanical hyperalgesia in rats following intraperitoneal (“IP”) dosing.

FIG. 3 depicts the effect of a TAAR1 agonist (compound 2) on SNL-induced mechanical hyperalgesia in rats following IP dosing.

FIG. 4 depicts the effect of a TAAR1 partial agonist (compound 3) on SNL-induced mechanical hyperalgesia in rats following PO dosing.

FIG. 5 depicts the effect of a TAAR1 partial agonist (compound 3) on SNL-induced mechanical hyperalgesia in rats following IP dosing.

FIG. 6 depicts the effect of a TAAR1 agonist (compound 2) on SNL-induced mechanical hyperalgesia in rats following PO dosing.

FIG. 7 depicts the effect of a TAAR1 agonist (compound 2) on SNL-induced mechanical hyperalgesia in 254 bp β-arrestin₂ KO rats following IP dosing.

FIG. 8 depicts the effect of a TAAR1 agonist (compound 2) on SNL-induced mechanical hyperalgesia in 1276 bp GRK5 KO rats following IP dosing.

FIG. 9 depicts the effect of a TAAR1 partial agonist (compound 1) on rotarod performance in rats.

FIG. 10 depicts the effect of a TAAR1 agonist (compound 2) on rotarod performance in rats.

FIG. 11 depicts the effect of a TAAR1 agonist (compound 2) on nociceptive pain or analgesia in the tail flick test.

FIG. 12 depicts the effect of a TAAR1 agonist (compound 2) on nociceptive pain or analgesia in the hot plate test.

### DETAILED DESCRIPTION OF THE INVENTION

The headings provided herein are not limitations of the various aspects of the disclosure, which can be defined by reference to the specification as a whole. Before describing the present invention in detail, it is to be understood that this invention is not limited to specific methods, compositions or steps, as such can vary.

### I. Definitions

As used in this application, except as otherwise expressly provided herein, each of the following terms shall have the meaning set forth below. Additional definitions are set forth throughout the application.

Unless defined otherwise, all other technical and scientific terms used herein have the same meaning as commonly understood by one of ordinary skill in the art to which this disclosure is related.

In this disclosure and the appended claims, the singular forms “a”, “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

The term “about” as used in connection with a numerical value throughout the disclosure and the claims denotes an interval of accuracy, familiar and acceptable to a person skilled in the art. Such interval of accuracy is +10%.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “aryl” refers to a monocyclic or bicyclic aromatic ring system having 6 to 14 carbon atoms. Non-limiting exemplary aryl groups include phenyl, naphthyl, phenanthryl, anthracyl, indenyl, azulenyl, biphenyl, biphenylenyl, and fluorenyl groups.

The terms “comprising” or “comprise” as used herein are synonymous with the terms “include,” “including,” “contain,” and “containing,” and are inclusive or open-ended and do not exclude additional, unrecited elements or method steps.

The term “cycloalkyl” refers a monocyclic or bicyclic alkylene ring system having 3 to 12 carbon ring atoms. Non-limiting exemplary cycloalkyl groups include cyclopropyl, cyclobutyl, cyclopentyl, cyclohexyl, cycloheptyl, cyclooctyl, and norbornyl.

The terms “enantiomer” and “enantiomeric” refer to a molecule that cannot be superimposed on its mirror image and hence is optically active wherein the enantiomer rotates the plane of polarized light in one direction and its mirror image compound rotates the plane of polarized light in the opposite direction.

The term “halogen” denotes chlorine, iodine, fluorine and bromine.

The term “heteroaryl” refers to a monocyclic or bicyclic aromatic ring system having 5 to 14 ring atoms, and one or more ring atoms are heteroatoms independently chosen from oxygen, nitrogen, and sulfur. Non-limiting exemplary heteroaryl groups include thienyl, thianthrenyl, furyl, benzofuryl, pyranyl, isobenzofuranyl, benzooxazonyl, pyrrolyl, imidazolyl, pyrazolyl, pyridyl, pyrazinyl, pyrimidinyl, pyridazinyl, isoindolyl, indolyl, indazolyl, thiazolyl, isothiazolyl, phenothiazolyl, isoxazolyl, furazanyl, and phenoxazinyl.

The term “heterocycloalkyl” is a monocyclic or bicyclic system having 3 to 12 ring atoms, and one or more of the ring atoms are heteroatoms independently chosen from oxygen, nitrogen, and sulfur.

The term “lower alkenyl” denotes an alkyl group as defined above containing one, two or three carbon-to-carbon double bonds. Non-limiting exemplary alkenyl groups include ethenyl, propenyl, isopropenyl, butenyl, sec-butenyl, pentenyl, and hexenyl.

The term “lower alkoxy” denotes a group wherein the alkyl residue is as defined above and which is attached via an oxygen atom.

The term “lower alkoxy substituted by halogen” denotes an alkoxy group as defined above, wherein at least one hydrogen atom is replaced by halogen, for example OCF₃, OCHF₂, OCH₂F, OCH₂CF₃, OCH₂CH₂CF₃, OCH₂CF₂CF₃, and the like.

The term “lower alkyl” denotes a saturated straight- or branched-chain group containing from 1 to 7 carbon atoms, for example, methyl, ethyl, propyl, isopropyl, n-butyl, i-butyl, 2-butyl, t-butyl, pentyl, hexyl, and the like. In certain embodiments of the disclosure, lower alkyl groups are alkyl groups with 1-4 carbon atoms.

The term “lower alkyl substituted by halogen” denotes an alkyl group as defined above, wherein at least one hydrogen atom is replaced by halogen, for example CF₃, CHF₂, CH₂F, CH₂CF₃, CH₂CH₂CF₃, CH₂CF₂CF₃, and the like.

The term “pharmaceutically acceptable,” such as a pharmaceutically acceptable carrier or excipient, etc., means pharmacologically acceptable and substantially non-toxic to the subject to which the particular compound is administered.

The term “pharmaceutically acceptable salts” embraces salts with inorganic and organic acids, and inorganic and organic bases. The pharmaceutically acceptable salts include, but are not limited to, metal salts such as sodium salt, potassium salt, and the like; alkaline earth metals such as calcium salt, magnesium salt, and the like; organic amine salts such as triethylamine salt, pyridine salt, picoline salt, ethanolamine salt, triethanolamine salt, dicyclohexylamine salt, N,N′-dibenzylethylenediamine salt, and the like; inorganic acid salts such as hydrochloride, hydrobromide, phosphate, sulphate, and the like; organic acid salts such as citrate, lactate, tartrate, maleate, fumarate, mandelate, acetate, dichloroacetate, trifluoroacetate, oxalate, formate, and the like; sulfonates such as methanesulfonate, benzenesulfonate, p-toluenesulfonate, and the like; and amino acid salts such as arginate, asparginate, glutamate, and the like.

The term “racemic” refers to a mixture of equal parts of enantiomers and which mixture is optically inactive.

The term “stereoisomers” is a general term for all isomers of individual molecules that differ only in the orientation of their atoms in space. It includes enantiomers and isomers of compounds with more than one chiral center that are not mirror images of one another (diastereomers).

The term “TAAR1 agonists” as used herein means compounds that bind to TAAR1 receptor and activates the receptor to produce a biological response, e.g., treating pain.

The term “TAAR1 partial agonists” as used herein means compounds that bind to a trace amine associated receptor 1 (“TAAR1”) receptor and activates the receptor to produce a biological response, but have only partial efficacy at the receptor relative to a full agonist. Criteria to assess whether or not a compound is a TAAR1 partial agonist is also known in the art (such as Revel et al., “Trace Amine-Associated Receptor 1 Partial Agonism Reveals Novel Paradigm for Neuropsychiatric Therapeutics,” Biol. Psychiatry 72:934-934 (2012)).

The term “treating” or “treatment” refers to partially or completely alleviating, ameliorating, improving, relieving, delaying onset of, inhibiting progression of, reducing severity of, preventing relapse of, and/or reducing incidence of one or more symptoms or features of a disease, e.g., pain.

Where ranges are given, endpoints are included. Furthermore, unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. The dosage amounts described herein are expressed in amounts of a compound in its free form, and do not include the weight of a counterion (e.g., sulfate) or any water or solvent molecules.

### II. TAAR1 Agonists or Partial Agonists

In one aspect, the present disclosure provides a method of treating or preventing pain in a subject, by administering to a subject in need thereof a therapeutically effective amount of a compound that is a TAAR1 agonist, a partial agonist, or a mixture thereof. In one embodiment, the pain to be treated or prevented is a chronic pain (e.g., neuropathic pain).

Methods to screen TAAR1 agonists or partial agonists are known in the art. See e.g., Tan et al., “Deciphering the Code to Aminergic G-Protein Coupled Receptor Drug Design,” Chem. Biol. 15(4):343-353 (2008); Reese et al., “Trace Amine-Associated Receptor 1 Displays Species-Dependent Stereoselectivity for Isomers of Methamphetamine, Amphetamine, and Para-Hydroxyamphetamine,” J. Pharmacology & Experimental Therapeutics 321:178-186 (2007).

TAAR1 agonists stimulate cAMP production in human embryonic kidney (HEK)-293 cells that express rTAAR1 in a concentration-dependent and saturable manner. EC₅₀, the effective concentration of an agonist that produces half of the maximal effect, is used as a measure of potency. In some embodiments, the TAAR1 agonists or partial agonists of the present disclosure have EC₅₀≤10 μm, or EC₅₀≤1 μm. An in vitro cAMP assay (agonist activity assay) is described below.

After incubating in fresh medium for at least 2 hours, HEK293 cells stably transfected with rTAAR1 are harvested in Krebs-Ringer-HEPES buffer (KRH) and preincubated with 200 μM 3-isobutyl-1-methylxanthine (IBMX) for 20-30 minutes. Cells are incubated in KRH with 133 μM IBMX and 3 μL of the test compound, forskolin (10 μM), or vehicle (dimethyl sulfoxide, DMSO) for 1 hour at 37° C. (300 μL total volume). The cells are boiled for 20 minutes after addition of 100 μL 0.5 mM sodium acetate buffer. The cell lysate is centrifuged to remove cellular debris, and an aliquot (30 μL) is transferred to an opaque, flat bottom 96-well plate (Corning #3917). The cAMP content of the aliquot is measured by use of the Hithunter™ cAMP XS kit (DiscoveRX, Fremont, Calif.). The plate is shaken on a titer plate shaker for 2 minutes after addition of 20 μL of cAMP XS antibody/lysis mix. After incubation in the dark for 1 hour, 20 μL of cAMP XS ED reagent is added and the plate is shaken for 2 minutes. After another hour of incubation in the dark, 40 μL of cAMP XS EA/CL substrate mix is added and the plate is shaken for 2 minutes. The plate is sealed with an acetate plate sealer (Thermo Scientific #3501) and allowed to incubate in the dark for 15-18 hours before luminescence is measured (3 readings/well at 0.33 seconds/reading) on an Analyst™ AD Assay Detection System (LJL Biosystems) or a Packard Fusion Microplate Reader.

The above method can be used to identify TAAR1 agonists that exhibit high binding affinities for TAAR1 with high potencies. An identical assay format has been used with hTAAR1 as well, expressed in CHO-K1 cells.

### III. Aminooxazoline Compounds

In one aspect, the present disclosure provides a method of treating pain in a subject, by administering to a subject in need thereof a therapeutically effective amount of a compound of Formula (I):

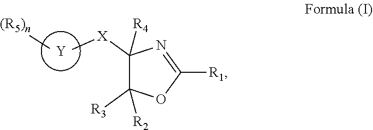

or a pharmaceutically acceptable salt, a racemic mixture, an enantiomer and/or optical isomer (stereoisomer), a tautomer, a prodrug, or a solvate thereof,
wherein:

R₁ is NH₂, NH(lower alkyl), or

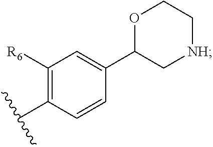

R₂ and R₃ are independently hydrogen or lower alkyl optionally substituted by halogen;

R₄ is hydrogen, phenyl, or lower alkyl;

R₅, each independently, is hydrogen, deuterium, tritium, cyano, halogen, lower alkyl optionally substituted by halogen, lower alkoxy optionally substituted by halogen, phenyl optionally substituted by halogen, phenyloxy, benzyl, benzyloxy, —C(O)O-lower alkyl, —NHC(O)-aryl wherein aryl is optionally substituted by lower alkyl or halogen, —O—(CH₂)ₒ—O-lower alkyl, —NH— cycloalkyl, cycloalkyl, piperidin-1-yl, or tetrahydropyran-4-yloxy, wherein the optional substituents for each R₅ are the same or different;

R₆ is hydrogen or halogen;

X is a bond, —(CHR)ₘ—, —O(CHR)ₘ—, —NRCHR′—, —CHROCHR′—, —SCHR—, —S(O)₂CH₂—, —CH₂SCH₂—, —CH₂N(R)CHR′—, -cycloalkyl-(CHR)ₘ—, or —SiRR′—CH₂—;

R and R′ are each independently hydrogen, lower alkyl optionally substituted by halogen, or benzyl optionally substituted by lower alkoxy or halogen, and when m>1, each R is the same or different;

Y is aryl, heteroaryl, cycloalkyl, or heterocycloalkyl;

m is 1, 2, 3, or 4;

n is 0, 1, 2, or 3; and

o is 2 or 3; and

wherein the compound is a TAAR1 agonist or partial agonist.

In one embodiment, the Y group is selected from the group of phenyl, naphthyl, thiophenyl, pyridinyl, cyclohexyl, 1,2,3,4-tetrahydro-naphthalen-2-yl, 2,3-dihydrobenzo[1,4]dioxin-6-yl, benzo[1,3]dioxol-5-yl, pyrimidyl, indanyl, 2,3-dihydroindol-1-yl, and 3,4-dihydro-quinolin-1-yl.

In one embodiment, R₂ and R₃ are, independently, hydrogen or C₁₋₇-alkyl optionally substituted by halogen; and R₄ is hydrogen, phenyl, or C₁₋₇-alkyl.

In another embodiment, R₅ is halogen, hydroxy, C₁₋₇-alkyl optionally substituted by halogen, C₁₋₇-alkoxy optionally substituted by halogen, phenyl optionally substituted by halogen, phenyloxy, benzyl, benzyloxy, —COO—C₁₋₇-alkyl, or —O—(CH₂)ₒ—O—C₁₋₇-alkyl, wherein o is 2 or 3.

In one embodiment, X is a bond, —CH₂—, —CH₂CH₂—, —CH₂CH₂CH₂—, —CH₂CH₂CH₂CH₂—, —OCH₂—, —CH₂O—, —CH₂CH₂O—, —CH₂OCH₂—, —CH(C₂H₅)CH₂—, —N(R)CH₂—,

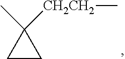

wherein R is hydrogen or C₁₋₇-alkyl.

In one embodiment, R₁ is NH₂, or

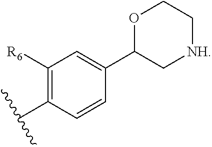

In one embodiment, R₁ is NH₂; R₂ and R₃ are hydrogen or C₁-C₇ alkyl; R₄ is hydrogen or C₁-C₇ alkyl; R₅ is hydrogen or halogen; X is a bond, —N(C₂H₅)CH₂—, or —CH(C₂H₅)CH₂—; and Y is phenyl.

In one embodiment, the compound of Formula (I) is of the structure:

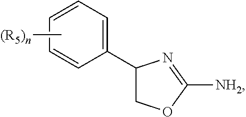

or a pharmaceutically acceptable salt, a racemic mixture, an enantiomer and/or optical isomer (stereoisomer), a tautomer, a prodrug, or a solvate thereof.

In one embodiment, the compound of Formula (I) is of the structure:

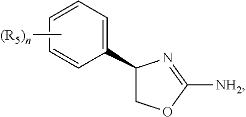

or a pharmaceutically acceptable salt, a racemic mixture, an enantiomer and/or optical isomer (stereoisomer), a tautomer, a prodrug, or a solvate thereof. In one embodiment, n is 1 or 2, and R₅, are each independently, is halogen or lower alkyl optionally substituted by halogen.

In one embodiment, the compound of Formula (I) is of the structure:

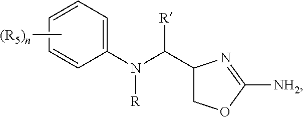

or a pharmaceutically acceptable salt, a racemic mixture, an enantiomer and/or optical isomer (stereoisomer), a tautomer, a prodrug, or a solvate thereof. In one embodiment, R and R′ are each independently hydrogen or lower alkyl optionally substituted by halogen.

In one embodiment, the compound of Formula (I) is of the structure:

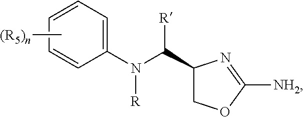

or a pharmaceutically acceptable salt, a racemic mixture, an enantiomer and/or optical isomer (stereoisomer), a tautomer, a prodrug, or a solvate thereof. In one embodiment, R and R′ are each independently hydrogen or lower alkyl optionally substituted by halogen.

In one embodiment, the compound of Formula (I) is of the structure:

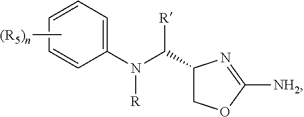

or a pharmaceutically acceptable salt, a racemic mixture, an enantiomer and/or optical isomer (stereoisomer), a tautomer, a prodrug, or a solvate thereof. In one embodiment, R and R′ are each independently hydrogen or lower alkyl optionally substituted by halogen.

In one embodiment, the compound of Formula (I) is of the structure:

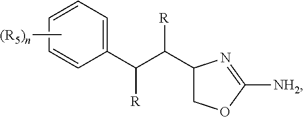

or a pharmaceutically acceptable salt, a racemic mixture, an enantiomer and/or optical isomer (stereoisomer), a tautomer, a prodrug, or a solvate thereof, wherein each R is the same or different.

In one embodiment, the compound of Formula (I) is of the structure:

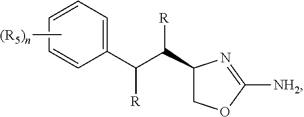

or a pharmaceutically acceptable salt, a racemic mixture, an enantiomer and/or optical isomer (stereoisomer), a tautomer, a prodrug, or a solvate thereof, wherein each R is the same or different.

In one embodiment, the compound of Formula (I) is 4-(3,4-dichlorophenyl)-4,5-dihydrooxazol-2-amine (compound 3), having the following structure:

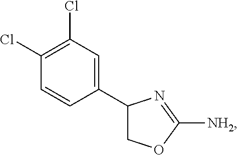 [SMILES: NC1=NC(c2ccc(Cl)c(Cl)c2)CO1]

or a pharmaceutically acceptable salt, a racemic mixture, an enantiomer and/or optical isomer (stereoisomer), a tautomer, a prodrug, or a solvate thereof.

In one embodiment, the compound of Formula (I) is (S)-4-(3,4-dichlorophenyl)-4,5-dihydrooxazol-2-amine, having the following structure:

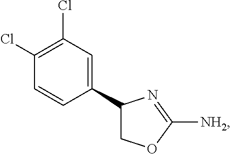

or a pharmaceutically acceptable salt, a racemic mixture, an enantiomer and/or optical isomer (stereoisomer), a tautomer, a prodrug, or a solvate thereof.

In one embodiment, the compound of Formula (I) is 4-((ethyl(phenyl)amino)methyl)-4,5-dihydrooxazol-2-amine (compound 2), having the following structure:

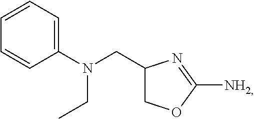 [SMILES: CCN(CC1COC(N)=N1)c1ccccc1]

or a pharmaceutically acceptable salt, a racemic mixture, an enantiomer and/or optical isomer (stereoisomer), a tautomer, a prodrug, or a solvate thereof.

In one embodiment, the compound of Formula (I) is of the structure:

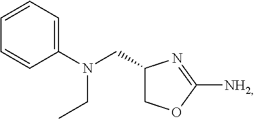 [SMILES: CCN(C[C@H]1COC(N)=N1)c1ccccc1]

or a pharmaceutically acceptable salt, a racemic mixture, an enantiomer and/or optical isomer (stereoisomer), a tautomer, a prodrug, or a solvate thereof.

In one embodiment, the compound of Formula (I) is 4-(3-fluoro-2-methylphenyl)-4,5-dihydrooxazol-2-amine, having the following structure:

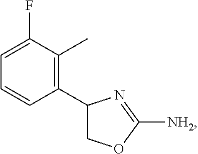

or a pharmaceutically acceptable salt, a racemic mixture, an enantiomer and/or optical isomer (stereoisomer), a tautomer, a prodrug, or a solvate thereof.

In one embodiment, the compound of Formula (I) is 4-(2-phenylbutyl)-4,5-dihydrooxazol-2-amine, having the following structure:

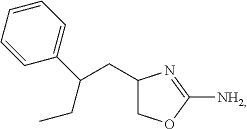 [SMILES: CCC(CC1COC(N)=N1)c1ccccc1]

or a pharmaceutically acceptable salt, a racemic mixture, an enantiomer and/or optical isomer (stereoisomer), a tautomer, a prodrug, or a solvate thereof.

In one embodiment, the compound of Formula (I) is 4-((1-(4-fluorophenyl)ethoxy)methyl)-4,5-dihydrooxazol-2-amine, having the following structure:

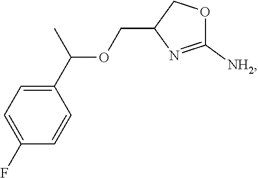 [SMILES: CC(OCC1COC(N)=N1)c1ccc(F)cc1]

or a pharmaceutically acceptable salt, a racemic mixture, an enantiomer and/or optical isomer (stereoisomer), a tautomer, a prodrug, or a solvate thereof.

In one embodiment, the compound of Formula (I) is N-(4-(2-(2-amino-4,5-dihydrooxazol-4-yl)ethyl)phenyl)-4-chlorobenzamide, having the following structure:

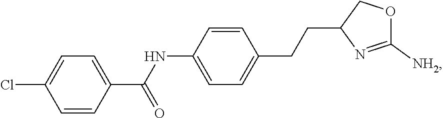 [SMILES: NC1=NC(CCc2ccc(NC(=O)c3ccc(Cl)cc3)cc2)CO1]

or a pharmaceutically acceptable salt, a racemic mixture, an enantiomer and/or optical isomer (stereoisomer), a tautomer, a prodrug, or a solvate thereof.

In another embodiment, the compound of Formula (I) is (S)—N-(4-(2-(2-amino-4,5-dihydrooxazol-4-yl)ethyl)phenyl)-4-chlorobenzamide, having the following structure:

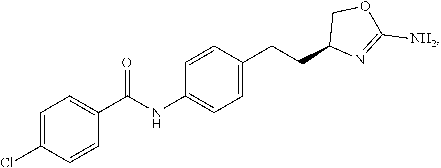 [SMILES: NC1=N[C@@H](CCc2ccc(NC(=O)c3ccc(Cl)cc3)cc2)CO1]

or a pharmaceutically acceptable salt, a racemic mixture, an enantiomer and/or optical isomer (stereoisomer), a tautomer, a prodrug, or a solvate thereof.

In another embodiment, the compound of Formula (I) is (S)-4-(3-fluoro-2-methylphenyl)-4,5-dihydrooxazol-2-amine, having the following structure:

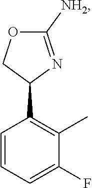

or a pharmaceutically acceptable salt, a racemic mixture, an enantiomer and/or optical isomer (stereoisomer), a tautomer, a prodrug, or a solvate thereof.

Some compounds disclosed herein contain one or more asymmetric centers and may thus give rise to enantiomers, diastereomers, and other stereoisomeric forms. The present methods also encompass the uses of all such possible forms, as well as their stereoisomeric, racemic, and resolved forms and mixtures thereof. All tautomers are also encompassed by the present methods.

Compounds of Formula (I) also include the following compounds:
(4R,5 S)-5-methyl-4-phenethyl-4,5-dihydro-oxazol-2-ylamine;
(4S,5 S)-5-methyl-4-phenethyl-4,5-dihydro-oxazol-2-ylamine;
(4S,5R)-5-methyl-4-phenethyl-4,5-dihydro-oxazol-2-ylamine;
(4S)-5-ethyl-4-phenethyl-4,5-dihydro-oxazol-2-ylamine;
(4S,5R)-5-methyl-4-(3-phenyl-propyl)-4,5-dihydro-oxazol-2-ylamine;
(4S,5 S)-5-methyl-4-(3-phenyl-propyl)-4,5-dihydro-oxazol-2-ylamine;
(4S,5 S)-4-benzyl-5-methyl-4,5-dihydro-oxazol-2-ylamine;
(4S,5R)-4-benzyl-5-methyl-4,5-dihydro-oxazol-2-ylamine;
(4S,5R)-4-[2-(4-chloro-phenyl)-ethyl]-5-methyl-4,5-dihydro-oxazol-2-ylamine;
(4S,5 S)-4-[2-(4-chloro-phenyl)-ethyl]-5-methyl-4,5-dihydro-oxazol-2-ylamine;
(4S,5R)-4-[2-(3,4-dichloro-phenyl)-ethyl]-5-methyl-4,5-dihydro-oxazol-2-ylamine;
(4S,5 S)-4-[2-(3,4-dichloro-phenyl)-ethyl]-5-methyl-4,5-dihydro-oxazol-2-ylamine;
(4S,5R)-4-[3-(4-chloro-phenyl)-propyl]-5-methyl-4,5-dihydro-oxazol-2-ylamine;
4-cyclohexyl-5-methyl-4,5-dihydro-oxazol-2-ylamine;
(4S,5 S)-4-(2-cyclohexyl-ethyl)-5-methyl-4,5-dihydro-oxazol-2-ylamine;
(4S,5R)-4-(2-cyclohexyl-ethyl)-5-methyl-4,5-dihydro-oxazol-2-ylamine;
(S)-4-((S)-2-phenyl-butyl)-4,5-dihydro-oxazol-2-ylamine;
(S)-4-(3-fluoro-2-methyl-phenyl)-4,5-dihydro-oxazol-2-ylamine;
(S)-4-(4-chloro-2-trifluoromethyl-phenyl)-4,5-dihydro-oxazol-2-ylamine;
3-[(S)-1-(S)-2-amino-4,5-dihydro-oxazol-4-ylmethyl)-propoxy]-phenol;
5-chloro-pyridine-2-carboxylic acid (4-pyrrolidin-3-yl-phenyl)-amide;
4-chloro-N-(4-pyrrolidin-3-yl-phenyl)-benzamide;
1-(5-chloro-pyridin-2-yl)-3-(4-pyrrolidin-3-yl-phenyl)urea;
(S)-4-[(S)-1-(4-fluoro-phenyl)-ethoxymethyl]-4,5-dihydro-oxazol-2-ylamine;
5-chloro-pyrimidine-2-carboxylic acid {4-[2-((S)-2-amino-4,5-dihydro-oxazol-4-yl)-ethyl]-phenyl}-amide;
N-{4-[2-((S)-2-amino-4,5-dihydro-oxazol-4-yl)-ethyl]-phenyl}-4-chlorobenzamide;
(R)-2-chloro-6-methyl-N-(4-(morpholin-2-yl)phenyl)isonicotinamide;
(S)—N-(4-(morpholin-2-yl)phenyl)-6-(2,2,2-trifluoroethoxy)nicotinamide;
(S)—N-(4-(morpholin-2-yl)phenyl)-2-(trifluoromethyl)isonicotinamide;
(S)-1-(4-fluorobenzyl)-3-(4-(morpholin-2-yl)phenyl)urea;
(S)-1-(3-cyanophenyl)-3-(4-(morpholin-2-yl)phenyl)urea;
(RS)-1-[4-(2-Amino-4,5-dihydro-oxazol-4-yl)-phenyl]-3-(4-chloro-phenyl)-urea;
(S)-6-chloro-N-(4-(morpholin-2-yl)phenyl)nicotinamide;
(R)-4-[(ethyl-phenyl-amino)-methyl]-4,5-dihydro-oxazol-2-ylamine;
(S)-4-[(ethyl-phenyl-amino)-methyl]-4,5-dihydro-oxazol-2-ylamine;
(S)-4-[(methyl-phenyl-amino)-methyl]-4,5-dihydro-oxazol-2-ylamine;
(S)-4-{[(3,4-dichloro-phenyl)-methyl-amino]-methyl}-4,5-dihydro-oxazol-2-ylamine;
(S)-4-{[(4-chloro-phenyl)-ethyl-amino]-methyl}-4,5-dihydro-oxazol-2-ylamine;
(S)-4-{[(3,4-dichloro-phenyl)-isopropyl-amino]-methyl}-4,5-dihydro-oxazol-2-ylamine;
(S)-4-{[(4-bromo-phenyl)-methyl-amino]-methyl}-4,5-dihydro-oxazol-2-ylamine;
(S)-4-{[(4-bromo-phenyl)-ethyl-amino]-methyl}-4,5-dihydro-oxazol-2-ylamine;
(S)-4-{[(3,4-dichloro-phenyl)-ethyl-amino]-methyl}-4,5-dihydro-oxazol-2-ylamine;
(S)-4-{[(3-bromo-phenyl)-methyl-amino]-methyl}-4,5-dihydro-oxazol-2-ylamine;
(S)-4-{[(3-bromo-phenyl)-ethyl-amino]-methyl}-4,5-dihydro-oxazol-2-ylamine;
(S)-4-{[(3-chloro-phenyl)-ethyl-amino]-methyl}-4,5-dihydro-oxazol-2-ylamine;
(S)-4-{[(4-chloro-2-fluoro-phenyl)-ethyl-amino]-methyl}-4,5-dihydro-oxazol-2-ylamine;
(S)-4-{[(4-chloro-2-fluoro-phenyl)-methyl-amino]-methyl}-4,5-dihydro-oxazol-2-ylamine;
(S)-4-{[ethyl-(2-fluoro-phenyl)-amino]-methyl}-4,5-dihydro-oxazol-2-ylamine;
(S)-4-{[(2-chloro-phenyl)-ethyl-amino]-methyl}-4,5-dihydro-oxazol-2-ylamine;
(R)-4-{[(4-chloro-phenyl)-methyl-amino]-methyl}-4,5-dihydro-oxazol-2-ylamine;
(R)-4-{[(4-chloro-phenyl)-ethyl-amino]-methyl}-4,5-dihydro-oxazol-2-ylamine;
(R)-4-{[(4-fluoro-phenyl)-methyl-amino]-methyl}-4,5-dihydro-oxazol-2-ylamine;
(R)-4-{[(4-chloro-phenyl)-isopropyl-amino]-methyl}-4,5-dihydro-oxazol-2-ylamine;
(S)-4-{[(2,4-difluoro-phenyl)-ethyl-amino]-methyl}-4,5-dihydro-oxazol-2-ylamine;
(S)-4-{[(2,4-difluoro-phenyl)-methyl-amino]-methyl}-4,5-dihydro-oxazol-2-ylamine;
(R)-4-{[(3,4-dichloro-phenyl)-methyl-amino]-methyl}-4,5-dihydro-oxazol-2-ylamine;
(R)-4-{[(3,4-dichloro-phenyl)-ethyl-amino]-methyl}-4,5-dihydro-oxazol-2-ylamine;
(R)-4-{[(3,4-dichloro-phenyl)-isopropyl-amino]-methyl}-4,5-dihydro-oxazol-2-ylamine;
(S)-4-{[(3,5-dichloro-phenyl)-methyl-amino]-methyl}-4,5-dihydro-oxazol-2-ylamine;
(S)-4-{[(3,5-dichloro-phenyl)-ethyl-amino]-methyl}-4,5-dihydro-oxazol-2-ylamine;
(S)-4-[(3-chloro-phenylamino)-methyl]-4,5-dihydro-oxazol-2-ylamine;
(S)-4-[(2-chloro-phenylamino)-methyl]-4,5-dihydro-oxazol-2-ylamine;
(S)-4-[(4-trifluoromethyl-phenylamino)-methyl]-4,5-dihydro-oxazol-2-ylamine;
(S)-4-[(2,4-difluoro-phenylamino)-methyl]-4,5-dihydro-oxazol-2-ylamine;
(S)-4-[(2-fluoro-4-methyl-phenylamino)-methyl]-4,5-dihydro-oxazol-2-ylamine;
(S)-4-{[ethyl-(4-trifluoromethyl-phenyl)-amino]-methyl}-4,5-dihydro-oxazol-2-ylamine;
(S)-4-{[methyl-(4-trifluoromethyl-phenyl)-amino]-methyl}-4,5-dihydro-oxazol-2-ylamine;
(S)-4-{[ethyl-(2-methyl-4-trifluoromethyl-phenyl)-amino]-methyl}-4,5-dihydro-oxazol-2-ylamine;
((S)-2-amino-4,5-dihydro-oxazol-4-ylmethyl)-(6-chloro-pyridin-2-yl)-amine;
(S)-4-{[(4-chloro-3-methoxy-phenyl)-methyl-amino]-methyl}-4,5-dihydro-oxazol-2-ylamine;
(S)-4-{[(4-chloro-3-methoxy-phenyl)-ethyl-amino]-methyl}-4,5-dihydro-oxazol-2-ylamine;
(S)-4-{[(4-fluoro-3-methoxy-phenyl)-methyl-amino]-methyl}-4,5-dihydro-oxazol-2-ylamine;
(R)-4-{[(4-fluoro-3-methoxy-phenyl)-methyl-amino]-methyl}-4,5-dihydro-oxazol-2-ylamine;
(R)-4-{[ethyl-(4-fluoro-3-methoxy-phenyl)-amino]-methyl}-4,5-dihydro-oxazol-2-ylamine;
(R)-4-{[(4-chloro-3-methoxy-phenyl)-methyl-amino]-methyl}-4,5-dihydro-oxazol-2-ylamine;
(R)-4-{[(4-chloro-3-methoxy-phenyl)-ethyl-amino]-methyl}-4,5-dihydro-oxazol-2-ylamine;
(S)-4-{[benzyl-(4-fluoro-3-methoxy-phenyl)-amino]-methyl}-4,5-dihydro-oxazol-2-ylamine;
(S)-4-{[benzyl-(4-fluoro-phenyl)-amino]-methyl}-4,5-dihydro-oxazol-2-ylamine;
(S)-4-{[benzyl-(4-chloro-phenyl)-amino]-methyl}-4,5-dihydro-oxazol-2-ylamine;
(S)-4-[(ethyl-m-tolyl-amino)-methyl]-4,5-dihydro-oxazol-2-ylamine;
(S)-4-{[ethyl-(3-ethyl-phenyl)-amino]-methyl}-4,5-dihydro-oxazol-2-ylamine;
(S)-4-{[ethyl-(4-ethyl-phenyl)-amino]-methyl}-4,5-dihydro-oxazol-2-ylamine;
(S)-4-[(methyl-naphthalen-2-yl-amino)-methyl]-4,5-dihydro-oxazol-2-ylamine,
(R)-4-[(methyl-naphthalen-2-yl-amino)-methyl]-4,5-dihydro-oxazol-2-ylamine;
(R)-4-[(ethyl-naphthalen-2-yl-amino)-methyl]-4,5-dihydro-oxazol-2-ylamine;
(S)-4-{[ethyl-(3-fluoro-5-trifluoromethyl-phenyl)-amino]-methyl}-4,5-dihydro-oxazol-2-ylamine;
(S)-4-[(3-fluoro-4-trifluoromethyl-phenylamino)-methyl]-4,5-dihydro-oxazol-2-ylamine;
(R)-4-{[ethyl-(3-fluoro-5-trifluoromethyl-phenyl)-amino]-methyl}-4,5-dihy dro-oxazol-2-ylamine;
(S)-4-[(ethyl-indan-5-yl-amino)-methyl]-4,5-dihydro-oxazol-2-ylamine;
(S)-4-{[methyl-(3-oxazol-5-yl-phenyl)-amino]-methyl}-4,5-dihydro-oxazol-2-ylamine;
(S)-4-{[ethyl-(3-oxazol-5-yl-phenyl)-amino]-methyl}-4,5-dihydro-oxazol-2-ylamine;
(4S)-4-(4-(1-(4-Chlorophenyl)-2,2,2-trifluoroethylamino)phenethyl)-4,5-dihydrooxazol-2-amine (1:1 mixture of epimers);
(+)—(S)-4-(4-((S)-1-(4-Chlorophenyl)-2,2,2-trifluoroethylamino)phenethyl)-4,5-dihydrooxazol-2-amine;
(−)-(S)-4-(4-((R)-1-(4-chlorophenyl)-2,2,2-trifluoroethylamino)phenethyl)-4,5-dihydrooxazol-2-amine;
(4S)-4-(4-(2,2,2-Trifluoro-1-(3-fluorophenyl)ethylamino)phenethyl)-4,5-dihydrooxazol-2-amine;
(4S)-4-(4-(2,2,2-Trifluoro-1-(4-(trifluoromethyl)phenyl)ethylamino)phenethyl)-4,5-dihydrooxazol-2-amine;
{5-[2-((S)-2-Amino-4,5-dihydro-oxazol-4-yl)-ethyl]-pyridin-2-yl}-(5-chloro-pyrimidin-2-yl)-amine;
1-{4-[2-((S)-2-amino-4,5-dihydro-oxazol-4-yl)-ethyl]-phenyl}-3-(4-chloro-phenyl)-urea;
N-{4-[3-((S)-2-amino-4,5-dihydro-oxazol-4-yl)-propyl]-phenyl}-4-chloro-benzamide;
N-{4-[2-((S)-2-amino-4,5-dihydro-oxazol-4-yl)-ethyl]-phenyl}-4-chloro-benzamide;
1-{4-[2-((S)-2-amino-4,5-dihydro-oxazol-4-yl)-ethyl]-phenyl}-3-(3,4-dichloro-phenyl)-urea;
1-{4-[2-((S)-2-amino-4,5-dihydro-oxazol-4-yl)-ethyl]-phenyl}-3-(4-trifluoromethyl-phenyl)-urea;
N-{4-[2-((S)-2-amino-4,5-dihydro-oxazol-4-yl)-ethyl]-phenyl}-4-fluoro-benzamide;
N-{4-[2-((S)-2-amino-4,5-dihydro-oxazol-4-yl)-ethyl]-phenyl}-4-trifluoromethyl-benzamide;
N-{4-[2-((S)-2-amino-4,5-dihydro-oxazol-4-yl)-ethyl]-phenyl}-3-chloro-benzamide;
N-{4-[2-((S)-2-amino-4,5-dihydro-oxazol-4-yl)-ethyl]-phenyl}-6-chloro-nicotinamide;
5-chloro-pyridine-2-carboxylic acid {4-[2-((S)-2-amino-4,5-dihydro-oxazol-4-yl)-ethyl]-phenyl}-amide;
5-chloro-pyrimidine-2-carboxylic acid {4-[2-((S)-2-amino-4,5-dihydro-oxazol-4-yl)-ethyl]-phenyl}-amide;
1-{4-[2-((S)-2-amino-4,5-dihydro-oxazol-4-yl)-ethyl]-phenyl}-3-(5-chloro-pyridin-2-yl)-urea;
1-{4-[2-((S)-2-amino-4,5-dihydro-oxazol-4-yl)-ethyl]-phenyl}-3-(6-chloro-pyridin-3-yl)-urea;
4,4-difluoro-cyclohexanecarboxylic acid {4-[2-((S)-2-amino-4,5-dihydro-oxazol-4-yl)-ethyl]-phenyl}-amide;
1-methyl-cyclopropanecarboxylic acid {4-[2-((S)-2-amino-4,5-dihydro-oxazol-4-yl)-ethyl]-phenyl}-amide;
cyclopentanecarboxylic acid {4-[2-((S)-2-amino-4,5-dihydro-oxazol-4-yl)-ethyl]-phenyl}-amide;
3,3-difluoro-cyclobutanecarboxylic acid {4-[2-((S)-2-amino-4,5-dihydro-oxazol-4-yl)-ethyl]-phenyl}-amide;
cyclobutanecarboxylic acid {4-[2-((S)-2-amino-4,5-dihydro-oxazol-4-yl)-ethyl]-phenyl}-amide;
cyclopropanecarboxylic acid {4-[2-((S)-2-amino-4,5-dihydro-oxazol-4-yl)-ethyl]-phenyl}-amide;
cyclohexanecarboxylic acid {4-[2-((S)-2-amino-4,5-dihydro-oxazol-4-yl)-ethyl]-phenyl}-amide;
1-trifluoromethyl-cyclopropanecarboxylic acid {4-[2-((S)-2-amino-4,5-dihydro-oxazol-4-yl)-ethyl]-phenyl}-amide;
1-(4-chloro-phenyl)-cyclopropanecarboxylic acid {4-[2-((S)-2-amino-4,5-dihydro-oxazol-4-yl)-ethyl]-phenyl}-amide;
5-trifluoromethyl-pyridine-2-carboxylic acid {4-[2-((S)-2-amino-4,5-dihydro-oxazol-4-yl)-ethyl]-phenyl}-amide;
N-{4-[2-((S)-2-amino-4,5-dihydro-oxazol-4-yl)-ethyl]-phenyl}-6-trifluoromethyl-nicotinamide;
1-{4-[2-((S)-2-amino-4,5-dihydro-oxazol-4-yl)-ethyl]-phenyl}-3-(4-fluoro-phenyl)-imidazolidin-2-one;
N-{4-[2-((S)-2-amino-4,5-dihydro-oxazol-4-yl)-ethyl]-phenyl}-2-(4-chloro-phenyl)-propionamide;
N-{4-[2-((S)-2-amino-4,5-dihydro-oxazol-4-yl)-ethyl]-phenyl}-4-cyano-benzamide;
N-{4-[2-((S)-2-amino-4,5-dihydro-oxazol-4-yl)-ethyl]-phenyl}-4-ethoxy-benzamide;
N-{4-[2-((S)-2-amino-4,5-dihydro-oxazol-4-yl)-ethyl]-phenyl}-4-propyl-benzamide;
N-{4-[2-((S)-2-amino-4,5-dihydro-oxazol-4-yl)-ethyl]-phenyl}-4-ethynyl-benzamide;
N-{4-[2-((S)-2-amino-4,5-dihydro-oxazol-4-yl)-ethyl]-phenyl}-4-methoxymethyl-benzamide;
N-{4-[2-((S)-2-amino-4,5-dihydro-oxazol-4-yl)-ethyl]-phenyl}-2-(4-ethoxy-phenyl)-acetamide;
N-{4-[2-((S)-2-amino-4,5-dihydro-oxazol-4-yl)-ethyl]-phenyl}-2-(4-methoxy-phenyl)-acetamide;
N-{4-[2-((S)-2-amino-4,5-dihydro-oxazol-4-yl)-ethyl]-phenyl}-2-(4-fluoro-phenyl)-acetamide;
N-{4-[2-((S)-2-amino-4,5-dihydro-oxazol-4-yl)-ethyl]-phenyl}-2-(4-chloro-phenyl)-acetamide;
N-{4-[2-((S)-2-amino-4,5-dihydro-oxazol-4-yl)-ethyl]-phenyl}-4-methoxy-benzamide;
N-{4-[2-((S)-2-amino-4,5-dihydro-oxazol-4-yl)-ethyl]-phenyl}-2-(4-chloro-phenyl)-isobutyramide;
N-{4-[2-((S)-2-amino-4,5-dihydro-oxazol-4-yl)-ethyl]-phenyl}-2-(4-bromo-phenyl)-2-methoxy-acetamide;
(S)—N-{4-[2-((S)-2-amino-4,5-dihydro-oxazol-4-yl)-ethyl]-phenyl}-2-methoxy-2-phenyl-acetamide;
N-{4-[2-((S)-2-amino-4,5-dihydro-oxazol-4-yl)-ethyl]-phenyl}-2-(4-chloro-phenyl)-2-methoxy-acetamide;
4-trifluoromethyl-cyclohexanecarboxylic acid {4-[2-((S)-2-amino-4,5-dihydro-oxazol-4-yl)-ethyl]-phenyl}-amide;
N-{4-[2-((S)-2-amino-4,5-dihydro-oxazol-4-yl)-ethyl]-phenyl}-3-(2-chloro-phenyl)-propionamide;
N-{4-[2-((S)-2-amino-4,5-dihydro-oxazol-4-yl)-ethyl]-phenyl}-2-(3-trifluoromethyl-phenyl)-propionamide;
N-{4-[2-((S)-2-amino-4,5-dihydro-oxazol-4-yl)-ethyl]-phenyl}-2-(3-trifluoromethoxy-phenyl)-propionamide;
2-methoxy-pyrimidine-5-carboxylic acid {4-[2-((S)-2-amino-4,5-dihydro-oxazol-4-yl)-ethyl]-phenyl}-amide;
N-{4-[2-((S)-2-amino-4,5-dihydro-oxazol-4-yl)-ethyl]-phenyl}-6-pyrazol-1-yl-nicotinamide;
1H-benzoimidazole-2-carboxylic acid {4-[2-((S)-2-amino-4,5-dihydro-oxazol-4-yl)-ethyl]-phenyl}-amide;
3,5-difluoro-pyridine-2-carboxylic acid {4-[2-((S)-2-amino-4,5-dihydro-oxazol-4-yl)-ethyl]-phenyl}-amide;
6-fluoro-pyridine-2-carboxylic acid {4-[2-((S)-2-amino-4,5-dihydro-oxazol-4-yl)-ethyl]-phenyl}-amide;
6-chloro-3-fluoro-pyridine-2-carboxylic acid {4-[2-((S)-2-amino-4,5-dihydro-oxazol-4-yl)-ethyl]-phenyl}-amide;
4-chloro-pyridine-2-carboxylic acid {4-[2-((S)-2-amino-4,5-dihydro-oxazol-4-yl)-ethyl]-phenyl}-amide;
quinoline-2-carboxylic acid {4-[2-((S)-2-amino-4,5-dihydro-oxazol-4-yl)-ethyl]-phenyl}-amide;
5-bromo-pyridine-2-carboxylic acid {4-[2-((S)-2-amino-4,5-dihydro-oxazol-4-yl)-ethyl]-phenyl}-amide;
isoquinoline-1-carboxylic acid {4-[2-((S)-2-amino-4,5-dihydro-oxazol-4-yl)-ethyl]-phenyl}-amide;
1-{4-[2-((S)-2-amino-4,5-dihydro-oxazol-4-yl)-ethyl]-phenyl}-3-(4-fluoro-phenyl)-urea;
1-{4-[2-((S)-2-amino-4,5-dihydro-oxazol-4-yl)-ethyl]-phenyl}-3-(3-chloro-phenyl)-urea;
(S)—N-(4-(2-(2-amino-4,5-dihydrooxazol-4-yl)ethyl)phenyl)-5-fluoropicolin-amide;
N-{4-[2-((S)-2-amino-4,5-dihydro-oxazol-4-yl)-ethyl]-phenyl}-6-methoxy-nicotinamide;
N-{4-[2-((S)-2-amino-4,5-dihydro-oxazol-4-yl)-ethyl]-phenyl}-6-methyl-nicotinamide;
(S)—N-(4-(2-(2-amino-4,5-dihydrooxazol-4-yl)ethyl)phenyl)-2-fluoronicotinamide;
N-{4-[2-((S)-2-amino-4,5-dihydro-oxazol-4-yl)-ethyl]-phenyl}-5-fluoro-nicotinamide;
3-chloro-5-trifluoromethyl-pyridine-2-carboxylic acid {4-[2-((S)-2-amino-4,5-dihydro-oxazol-4-yl)-ethyl]-phenyl}-amide;
[1,6]naphthyridine-2-carboxylic acid {4-[2-((S)-2-amino-4,5-dihydro-oxazol-4-yl)-ethyl]-phenyl}-amide;
[1,8]naphthyridine-2-carboxylic acid {4-[2-((S)-2-amino-4,5-dihydro-oxazol-4-yl)-ethyl]-phenyl}-amide;
N-{4-[2-((S)-2-amino-4,5-dihydro-oxazol-4-yl)-ethyl]-phenyl}-6-bromo-nicotinamide;
N-{4-[2-((S)-2-amino-4,5-dihydro-oxazol-4-yl)-ethyl]-phenyl}-2,5-difluoro-nicotinamide;
N-{4-[2-((S)-2-amino-4,5-dihydro-oxazol-4-yl)-ethyl]-phenyl}-5,6-dichloro-nicotinamide;
N-{4-[2-((S)-2-amino-4,5-dihydro-oxazol-4-yl)-ethyl]-phenyl}-2,6-difluoro-nicotinamide;
N-{4-[2-((S)-2-amino-4,5-dihydro-oxazol-4-yl)-ethyl]-phenyl}-6-cyano-nicotinamide;
6-bromo-pyridine-2-carboxylic acid {4-[2-((S)-2-amino-4,5-dihydro-oxazol-4-yl)-ethyl]-phenyl}-amide;
N-{4-[2-((S)-2-amino-4,5-dihydro-oxazol-4-yl)-ethyl]-phenyl}-6-(2,2,2-trifluoro-ethoxy)-nicotinamide;
N-{4-[2-((S)-2-amino-4,5-dihydro-oxazol-4-yl)-ethyl]-phenyl}-2-methoxy-nicotinamide;
(S)—N-(4-(2-(2-amino-4,5-dihydrooxazol-4-yl)ethyl)phenyl)-5-methoxypicolinamide;
(S)—N-(4-(2-(2-amino-4,5-dihydrooxazol-4-yl)ethyl)phenyl)-5-cyanopicolinamide;
(S)—N-(4-(2-(2-amino-4,5-dihydrooxazol-4-yl)ethyl)phenyl)-6-fluoronicotinamide;
(S)—N-(4-(2-(2-amino-4,5-dihydrooxazol-4-yl)ethyl)phenyl)-6-chloropyridazine-3-carboxamide;
(S)—N-(4-(2-(2-amino-4,5-dihydrooxazol-4-yl)ethyl)phenyl)-2-(piperidin-1-yl)pyrimidine-5-carboxamide;
(S)—N-(4-(2-(2-amino-4,5-dihydrooxazol-4-yl)ethyl)phenyl)-5-chloronicotinamide;
(S)—N-(4-(2-(2-amino-4,5-dihydrooxazol-4-yl)ethyl)phenyl)-2-(3,4-dichlorophenyl)-2,2-difluoroacetamide;
(S)—N-(4-(2-(2-amino-4,5-dihydrooxazol-4-yl)ethyl)phenyl)-5-chloropyrazine-2-carboxamide;
(S)—N-(4-(2-(2-amino-4,5-dihydrooxazol-4-yl)ethyl)phenyl)-5-methoxypyrazine-2-carboxamide;
(S)—N-(4-(2-(2-amino-4,5-dihydrooxazol-4-yl)ethyl)phenyl)-6-methoxypyrazine-2-carboxamide;
(S)—N-(4-(2-(2-amino-4,5-dihydrooxazol-4-yl)ethyl)phenyl)-5-methylpyrazine-2-carboxamide;
(S)-4-(2-(2-amino-4,5-dihydrooxazol-4-yl)ethyl)-N-(5-chloropyridin-2-yl)benzamide;
(S)—N-(4-(2-(2-amino-4,5-dihydrooxazol-4-yl)ethyl)phenyl)-4-chloro-2-fluorobenzamide;
(S)—N-(4-(2-(2-amino-4,5-dihydrooxazol-4-yl)ethyl)phenyl)-2,4-dichlorobenzamide;
(S)—N-(4-(2-(2-amino-4,5-dihydrooxazol-4-yl)ethyl)phenyl)-4-chloro-2-methoxybenzamide;
(S)-4-(2-(2-amino-4,5-dihydrooxazol-4-yl)ethyl)-N-(4-cyanophenyl)benzamide;
(S)-4-(2-(2-amino-4,5-dihydrooxazol-4-yl)ethyl)-N-(4-ethynylphenyl)benzamide;
{4-[2-((S)-2-amino-4,5-dihydro-oxazol-4-yl)-ethyl]-phenyl}-carbamic acid 4-chloro-benzyl ester;
{4-[2-((S)-2-amino-4,5-dihydro-oxazol-4-yl)-ethyl]-phenyl}-carbamic acid 4-methoxy-phenyl ester;
{4-[2-((S)-2-amino-4,5-dihydro-oxazol-4-yl)-ethyl]-phenyl}-carbamic acid 4-fluoro-phenyl ester;
{4-[2-((S)-2-amino-4,5-dihydro-oxazol-4-yl)-ethyl]-phenyl}-carbamic acid 3-trifluoromethyl-phenyl ester;
1-{4-[2-((S)-2-amino-4,5-dihydro-oxazol-4-yl)-ethyl]-phenyl}-3-(5-chloro-pyrimidin-2-yl)-urea;
(S)—N-(4-(2-(2-amino-4,5-dihydrooxazol-4-yl)ethyl)phenyl)-5-ethoxypicolinamide;
(S)—N-(4-(2-(2-amino-4,5-dihydrooxazol-4-yl)ethyl)phenyl)-5-chloro-4-methylpicolinamide;
(S)—N-(4-(2-(2-amino-4,5-dihydrooxazol-4-yl)ethyl)phenyl)-4-chloro-6-methylpicolinamide;
(S)—N-(4-(2-(2-amino-4,5-dihydrooxazol-4-yl)ethyl)phenyl)-4,6-dimethylpicolinamide;
(S)—N-(4-(2-(2-amino-4,5-dihydrooxazol-4-yl)ethyl)phenyl)-4,6-dichloropicolinamide;
(S)—N-(4-(2-(2-amino-4,5-dihydrooxazol-4-yl)ethyl)phenyl)-5-(trifluoromethyl)pyrazine-2-carboxamide;
(S)—N-(4-(2-(2-amino-4,5-dihydrooxazol-4-yl)ethyl)phenyl)-6-chloropyrazine-2-carboxamide;
(S)—N-(4-(2-(2-amino-4,5-dihydrooxazol-4-yl)ethyl)phenyl)-5-ethoxypyrazine-2-carboxamide;
(S)—N-(4-(2-(2-amino-4,5-dihydrooxazol-4-yl)ethyl)phenyl)-5-morpholinopyrazine-2-carboxamide;
(S)—N-(4-(2-(2-amino-4,5-dihydrooxazol-4-yl)ethyl)phenyl)-6-chloro-5-methoxypicolinamide;
(S)—N-(4-(2-(2-amino-4,5-dihydrooxazol-4-yl)ethyl)phenyl)-5-chlorothiophene-2-carboxamide;
(S)—N-(4-(2-(2-amino-4,5-dihydrooxazol-4-yl)ethyl)phenyl)-3-cyclopropylpropanamide;
(S)—N-(4-(2-(2-amino-4,5-dihydrooxazol-4-yl)ethyl)phenyl)-5-methylpicolinamide;
(S)—N-(4-(2-(2-amino-4,5-dihydrooxazol-4-yl)ethyl)phenyl)-2-methylisonicotinamide;
(S)—N-(4-(2-(2-amino-4,5-dihydrooxazol-4-yl)ethyl)phenyl)-2-chloroisonicotinamide;
(S)—N-(4-(2-(2-amino-4,5-dihydrooxazol-4-yl)ethyl)phenyl)-2-chloro-3-fluoroisonicotinamide;
(S)—N-(4-(2-(2-amino-4,5-dihydrooxazol-4-yl)ethyl)phenyl)-2,6-dichloroisonicotinamide;
(S)—N-(4-(2-(2-amino-4,5-dihydrooxazol-4-yl)ethyl)phenyl)-5-ethylpicolinamide;
(S)—N-(4-(2-(2-amino-4,5-dihydrooxazol-4-yl)ethyl)phenyl)-2,5-dimethyloxazole-4-carboxamide;
(S)—N-(4-(2-(2-amino-4,5-dihydrooxazol-4-yl)ethyl)phenyl)-2-(dimethylamino)-5-isopropylthiazole-4-carboxamide;
(S)—N-(4-(2-(2-amino-4,5-dihydrooxazol-4-yl)ethyl)phenyl)-2,6-dimethoxypyrimidine-4-carboxamide;
(S)—N-(4-(2-(2-amino-4,5-dihydrooxazol-4-yl)ethyl)phenyl)-5-methyloxazole-4-carboxamide;
(S)—N-(4-(2-(2-amino-4,5-dihydrooxazol-4-yl)ethyl)phenyl)-4-chloro-1-methyl-1H-pyrazole-3-carboxamide;
(S)—N-(4-(2-(2-amino-4,5-dihydrooxazol-4-yl)ethyl)phenyl)-1-(difluoromethyl)-1H-pyrazole-3-carboxamide;
(S)—N-(4-(2-(2-amino-4,5-dihydrooxazol-4-yl)ethyl)phenyl)-2-tert-butylisonicotinamide;
(S)—N-(4-(2-(2-amino-4,5-dihydrooxazol-4-yl)ethyl)phenyl)-2-isopropylisonicotinamide;
(S)—N-(4-(2-(2-amino-4,5-dihydrooxazol-4-yl)ethyl)phenyl)-2,4′-bipyridine-4-carboxamide;
(S)—N-(4-(2-(2-amino-4,5-dihydrooxazol-4-yl)ethyl)phenyl)-5-methylthiophene-2-carboxamide;
(S)—N-(4-(2-(2-amino-4,5-dihydrooxazol-4-yl)ethyl)phenyl)-4,5-dimethylthiophene-2-carboxamide;
2-ethyl-6-methoxy-pyrimidine-4-carboxylic acid {4-[2-((S)-2-amino-4,5-dihydro-oxazol-4-yl)-ethyl]-phenyl}-amide;
N-(4-(2-((4S,5 S)-2-amino-5-methyl-4,5-dihydrooxazol-4-yl)ethyl)phenyl)-4-chlorobenzamide;
N-(4-(2-((4S,5R)-2-amino-5-methyl-4,5-dihydrooxazol-4-yl)ethyl)phenyl)-4-chlorobenzamide;
2-isopropyl-6-methoxy-pyrimidine-4-carboxylic acid {4-[2-((S)-2-amino-4,5-dihydro-oxazol-4-yl)-ethyl]-phenyl}-amide;
(S)—N-(4-(2-(2-amino-4,5-dihydrooxazol-4-yl)ethyl)phenyl)-5-ethylpyrazine-2-carboxamide;
(S)—N-(4-(2-(2-amino-4,5-dihydrooxazol-4-yl)ethyl)phenyl)-4-cyclopropyl-2-(trifluoromethyl)pyrimidine-5-carboxamide;
(S)—N-(4-(2-(2-amino-4,5-dihydrooxazol-4-yl)ethyl)phenyl)-6-ethylpyrazine-2-carboxamide;
(S)—N-(4-(2-(2-amino-4,5-dihydrooxazol-4-yl)ethyl)phenyl)-5-ethyloxazole-4-carboxamide;
(S)—N-(4-(2-(2-amino-4,5-dihydrooxazol-4-yl)ethyl)phenyl)-5-cyclopropyloxazole-4-carboxamide;
(S)—N-(4-(2-(2-amino-4,5-dihydrooxazol-4-yl)ethyl)phenyl)-5-isopropyloxazole-4-carboxamide;
(S)—N-(4-(2-(2-amino-4,5-dihydrooxazol-4-yl)ethyl)phenyl)-2-methyl-5-(trifluoromethyl)oxazole-4-carboxamide;
(S)—N-(4-(2-(2-amino-4,5-dihydrooxazol-4-yl)ethyl)phenyl)-2-ethyloxazole-4-carboxamide;
5-ethoxy-pyrazine-2-carboxylic acid {4-[2-((4S,5S)-2-amino-5-methyl-4,5-dihydro-oxazol-4-yl)-ethyl]-phenyl}-amide;
2-chloro-6-methoxy-pyrimidine-4-carboxylic acid {4-[2-((4S,5S)-2-amino-5-methyl-4,5-dihydro-oxazol-4-yl)-ethyl]-phenyl}-amide;
5-methyl-oxazole-4-carboxylic acid {4-[2-((4S,5S)-2-amino-5-methyl-4,5-dihydro-oxazol-4-yl)-ethyl]-phenyl}-amide;
5-trifluoromethyl-pyridine-2-carboxylic acid {4-[2-((4S,5S)-2-amino-5-methyl-4,5-dihydro-oxazol-4-yl)-ethyl]-phenyl}-amide;
5-ethyl-pyrazine-2-carboxylic acid {4-[2-((4S,5S)-2-amino-5-methyl-4,5-dihydro-oxazol-4-yl)-ethyl]-phenyl}-amide;
(S)—N-(4-(2-(2-amino-4,5-dihydrooxazol-4-yl)ethyl)phenyl)-6-isopropylpyrazine-2-carboxamide;
2-chloro-6-methoxy-pyrimidine-4-carboxylic acid {4-[2-((S)-2-amino-4,5-dihydro-oxazol-4-yl)-ethyl]-phenyl}-amide;
1-{4-[(S)-1-((S)-2-amino-4,5-dihydro-oxazol-4-ylmethyl)-propyl]-phenyl}-3-(4-fluoro-phenyl)-urea;
1-{4-[(S)-1-((S)-2-amino-4,5-dihydro-oxazol-4-ylmethyl)-propyl]-phenyl}-3-(4-trifluoromethyl-phenyl)-urea;
N-{4-[(R)-1-((S)-2-amino-4,5-dihydro-oxazol-4-ylmethyl)-2,2,2-trifluoro-ethoxy]-phenyl}-4-chloro-benzamide;
(S)-4-(2-chloro-phenyl)-4,5-dihydro-oxazol-2-ylamine;
(RS)-4-(4-chloro-phenyl)-4,5-dihydro-oxazol-2-ylamine;
(RS)-4-(2,4-difluoro-phenyl)-4,5-dihydro-oxazol-2-ylamine;
(S)-4-(2,3-difluoro-phenyl)-4,5-dihydro-oxazol-2-ylamine;
(RS)-4-(3,4-dichlorophenyl)-4,5-dihydro-oxazol-2-ylamine;
(RS)-4-(2,5-difluoro-phenyl)-4,5-dihydro-oxazol-2-ylamine;
(RS)-4-(2,3,4-trifluoro-phenyl)-4,5-dihydro-oxazol-2-ylamine;
(RS)-4-(3-chloro-2-fluoro-phenyl)-4,5-dihydro-oxazol-2-ylamine;
(RS)-4-(5-chloro-2-fluoro-phenyl)-4,5-dihydro-oxazol-2-ylamine;
(RS)-4-(4-bromo-phenyl)-4,5-dihydro-oxazol-2-ylamine;
(S)-4-(3-bromo-2,4-difluoro-phenyl)-4,5-dihydro-oxazol-2-ylamine;
(S)-4-(2,4-difluoro-phenyl)-4,5-dihydro-oxazol-2-ylamine;
(S)-4-(3,4-dichloro-phenyl)-4,5-dihydro-oxazol-2-ylamine;
(S)-4-(2,5-difluoro-phenyl)-4,5-dihydro-oxazol-2-ylamine;
(S)-4-(4-bromo-phenyl)-4,5-dihydro-oxazol-2-ylamine;
(S)-4-(2,4,5-trifluoro-phenyl)-4,5-dihydro-oxazol-2-ylamine;
(S)-4-(3-chloro-2-fluoro-phenyl)-4,5-dihydro-oxazol-2-ylamine;
(S)-4-(2,3,5-trifluoro-phenyl)-4,5-dihydro-oxazol-2-ylamine;
(S)-4-(3,4-dichloro-phenyl)-4-methyl-4,5-dihydro-oxazol-2-ylamine;
(S)-4-(4-chloro-phenyl)-4-methyl-4,5-dihydro-oxazol-2-ylamine;
(S)-4-(3,4-difluoro-phenyl)-4,5-dihydro-oxazol-2-ylamine;
(S)-4-(2,3-dichloro-phenyl)-4,5-dihydro-oxazol-2-ylamine;
(S)-4-(4-chloro-phenyl)-4,5-dihydro-oxazol-2-ylamine;
(S)-4-(3,4,5-trifluoro-phenyl)-4,5-dihydro-oxazol-2-ylamine;
(S)-4-(3-chloro-4-fluoro-phenyl)-4,5-dihydro-oxazol-2-ylamine;
(S)-4-(4-chloro-3-fluoro-phenyl)-4,5-dihydro-oxazol-2-ylamine;
(S)-4-(3,5-difluoro-phenyl)-4,5-dihydro-oxazol-2-ylamine;
(S)-4-(3-chloro-5-fluoro-phenyl)-4,5-dihydro-oxazol-2-ylamine;
(S)-4-(2-chloro-phenyl)-4-methyl-4,5-dihydro-oxazol-2-ylamine;
(RS)-4-(3-chloro-phenyl)-4,5-dihydro-oxazol-2-ylamine;
(RS)-4-(3-bromo-phenyl)-4,5-dihydro-oxazol-2-ylamine;
(S)-4-(2,5-difluoro-phenyl)-4-methyl-4,5-dihydro-oxazol-2-ylamine;
(RS)-4-(3-fluoro-phenyl)-4,5-dihydro-oxazol-2-ylamine;
(RS)-4-(3,5-dichloro-phenyl)-4,5-dihydro-oxazol-2-ylamine;
(+)-(S)-4-(5-chloro-2-fluoro-phenyl)-4,5-dihydro-oxazol-2-ylamine;
(+)-(S)-4-(3-chloro-phenyl)-4,5-dihydro-oxazol-2-ylamine;
(S)-4-(2-fluoro-phenyl)-4-methyl-4,5-dihydro-oxazol-2-ylamine;
(+)-(S)-4-(3-bromo-phenyl)-4,5-dihydro-oxazol-2-ylamine;
(+)-(S)-4-(3-fluoro-phenyl)-4,5-dihydro-oxazol-2-ylamine;
(+)-(S)-4-(3,5-dichloro-phenyl)-4,5-dihydro-oxazol-2-ylamine;
(S)-4-(2,4-difluoro-phenyl)-4-methyl-4,5-dihydro-oxazol-2-ylamine;
(RS)-4-methyl-4-(2,3,5-trifluoro-phenyl)-4,5-dihydro-oxazol-2-ylamine;
(S)-4-(4-bromo-phenyl)-4-methyl-4,5-dihydro-oxazol-2-ylamine;
(RS)-4-(2,4-dichloro-phenyl)-4,5-dihydro-oxazol-2-ylamine;
(S)-4-(4-chloro-3-fluoro-phenyl)-4-methyl-4,5-dihydro-oxazol-2-ylamine;
(RS)-4-(2-bromo-phenyl)-4,5-dihydro-oxazol-2-ylamine;
(RS)-4-methyl-4-(2,4,5-trifluoro-phenyl)-4,5-dihydro-oxazol-2-ylamine;
(RS)-4-(4-bromo-3-fluoro-phenyl)-4,5-dihydro-oxazol-2-ylamine;
(RS)-4-(2,5-dichloro-phenyl)-4,5-dihydro-oxazol-2-ylamine;
(+)-(S)-4-(2-bromo-phenyl)-4,5-dihydro-oxazol-2-ylamine;
(S)-4-(3-chloro-5-fluoro-phenyl)-4-methyl-4,5-dihydro-oxazol-2-ylamine;
(RS)-4-methyl-4-(2,3,4-trifluoro-phenyl)-4,5-dihydro-oxazol-2-ylamine;
(S)-4-(5-chloro-2-fluoro-phenyl)-4-methyl-4,5-dihydro-oxazol-2-ylamine;
(RS)-4-methyl-4-(4-chloro-2,5-difluoro-phenyl)-4,5-dihydro-oxazol-2-ylamine;
(RS)-4-methyl-4-(3,4,5-trifluoro-phenyl)-4,5-dihydro-oxazol-2-ylamine;
(S)-4-(3-chloro-4-fluoro-phenyl)-4-methyl-4,5-dihydro-oxazol-2-ylamine;
(S)-4-(4-chloro-2,5-difluoro-phenyl)-4-methyl-4,5-dihydro-oxazol-2-ylamine;
(RS)-4-(4-bromo-2-chloro-phenyl)-4,5-dihydro-oxazol-2-ylamine;
(RS)-4-(2,4-dichloro-phenyl)-4-methyl-4,5-dihydro-oxazol-2-ylamine;
(−)-(R)-4-(2,4-dichloro-phenyl)-4,5-dihydro-oxazol-2-ylamine;
(+)-(S)-4-(2,4-dichloro-phenyl)-4,5-dihydro-oxazol-2-ylamine;
(+)-(S)-4-(2,5-dichloro-phenyl)-4,5-dihydro-oxazol-2-ylamine;
(RS)-4-(2,5-dichloro-phenyl)-4-methyl-4,5-dihydro-oxazol-2-ylamine;
(+)-(S)-4-(2,3,4-trifluoro-phenyl)-4,5-dihydro-oxazol-2-ylamine;
(S)-4-methyl-4-(3,4,5-trifluoro-phenyl)-4,5-dihydro-oxazol-2-ylamine;
(RS)-4-(3-chloro-2-fluoro-phenyl)-4-methyl-4,5-dihydro-oxazol-2-ylamine;
(S)-4-(4-chloro-phenyl)-4-ethyl-4,5-dihydro-oxazol-2-ylamine;
(−)-(R)-4-(4-bromo-2-chloro-phenyl)-4,5-dihydro-oxazol-2-ylamine;
(+)-(S)-4-(4-bromo-2-chloro-phenyl)-4,5-dihydro-oxazol-2-ylamine;
(RS)-4-(2-chloro-4-fluoro-phenyl)-4-methyl-4,5-dihydro-oxazol-2-ylamine;
(+)-(S)-4-(4-bromo-3-fluoro-phenyl)-4,5-dihydro-oxazol-2-ylamine;
(RS)-4-(4-bromo-3-chloro-phenyl)-4,5-dihydro-oxazol-2-ylamine;
(S)-4-(3,4-dichloro-phenyl)-4-ethyl-4,5-dihydro-oxazol-2-ylamine;
(+)-(S)-4-(4-bromo-3-chloro-phenyl)-4,5-dihydro-oxazol-2-ylamine;
(RS)-4-(4-bromo-2-fluoro-phenyl)-4,5-dihydro-oxazol-2-ylamine;
(S)-4-(4-bromo-2-fluoro-phenyl)-4,5-dihydro-oxazol-2-ylamine,
(RS)-4-(2-trifluoromethyl-phenyl)-4,5-dihydro-oxazol-2-ylamine;
(RS)-4-o-tolyl-4,5-dihydro-oxazol-2-ylamine;
(RS)-4-(4-trifluoromethoxy-phenyl)-4,5-dihydro-oxazol-2-ylamine;
(RS)-4-(4-methoxy-3-methyl-phenyl)-4,5-dihydro-oxazol-2-ylamine;
(S)-4-o-tolyl-4,5-dihydro-oxazol-2-ylamine;
(RS)-4-(4-benzyloxy-phenyl)-4,5-dihydro-oxazol-2-ylamine;
(S)-4-(2-trifluoromethyl-phenyl)-4,5-dihydro-oxazol-2-ylamine;
(S)-4-(4-methoxy-3-methyl-phenyl)-4,5-dihydro-oxazol-2-ylamine;
(S)-4-methyl-4-(4-trifluoromethyl-phenyl)-4,5-dihydro-oxazol-2-ylamine;
(S)-4-(3-trifluoromethyl-phenyl)-4,5-dihydro-oxazol-2-ylamine;
(S)-4-methyl-4-p-tolyl-4,5-dihydro-oxazol-2-ylamine;
(S)-4-methyl-4-(3-trifluoromethyl-phenyl)-4,5-dihydro-oxazol-2-ylamine;
(RS)-4-(4-methoxy-2-trifluoromethyl-phenyl)-4,5-dihydro-oxazol-2-ylamine;
(+)-(S)-4-(4-methoxy-2-trifluoromethyl-phenyl)-4,5-dihydro-oxazol-2-ylamine;
(RS)-4-biphenyl-4-yl-4,5-dihydro-oxazol-2-ylamine;
(S)-4-biphenyl-4-yl-4,5-dihydro-oxazol-2-ylamine;
(RS)-4-(4′-chloro-biphenyl-4-yl)-4,5-dihydro-oxazol-2-ylamine;
(RS)-4-(3-chloro-4-methoxyphenyl)-4,5-dihydro-oxazol-2-ylamine;
(S)-4-(3-chloro-4-methoxy-phenyl)-4,5-dihydro-oxazol-2-ylamine;
(S)-4-(2-fluoro-4-trifluoromethyl-phenyl)-4-methyl-4,5-dihydro-oxazol-2-ylamine;
(RS)-4-(4-fluoro-2-trifluoromethyl-phenyl)-4,5-dihydro-oxazol-2-ylamine;
(RS)-4-(5-chloro-2-trifluoromethyl-phenyl)-4,5-dihydro-oxazol-2-ylamine;
(RS)-4-(4-chloro-2-trifluoromethyl-phenyl)-4,5-dihydro-oxazol-2-ylamine;
(RS)-4-(4,5-dichloro-2-trifluoromethyl-phenyl)-4,5-dihydro-oxazol-2-ylamine;
(+)-(S)-4-(4-chloro-2-trifluoromethyl-phenyl)-4,5-dihydro-oxazol-2-ylamine;
(+)-(S)-4-(4,5-dichloro-2-trifluoromethyl-phenyl)-4,5-dihydro-oxazol-2-ylamine;
(−)-(R)-4-(4,5-dichloro-2-trifluoromethyl-phenyl)-4,5-dihydro-oxazol-2-ylamine;
(+)-(S)-4-(4-fluoro-2-trifluoromethyl-phenyl)-4,5-dihydro-oxazol-2-ylamine;
(RS)-4-(5-fluoro-2-trifluoromethyl-phenyl)-4,5-dihydro-oxazol-2-ylamine;
(RS)-4-(5-fluoro-2-methyl-phenyl)-4,5-dihydro-oxazol-2-ylamine;
(+)-(S)-4-(5-fluoro-2-trifluoromethyl-phenyl)-4,5-dihydro-oxazol-2-ylamine;
(RS)-4-(4-chloro-3-methyl-phenyl)-4-methyl-4,5-dihydro-oxazol-2-ylamine;
(RS)-4-(5-chloro-2-methyl-phenyl)-4,5-dihydro-oxazol-2-ylamine;
(RS)-4-(4-chloro-2-fluoro-5-methyl-phenyl)-4-methyl-4,5-dihydro-oxazol-2-ylamine;
(S)-4-(4-chloro-3-methyl-phenyl)-4-methyl-4,5-dihydro-oxazol-2-ylamine;
(RS)-4-(4-bromo-2-trifluoromethyl-phenyl)-4,5-dihydro-oxazol-2-ylamine;
(S)-4-(4-chloro-2-fluoro-5-methyl-phenyl)-4-methyl-4,5-dihydro-oxazol-2-ylamine;
(+)-(S)-4-(5-chloro-2-methyl-phenyl)-4,5-dihydro-oxazol-2-ylamine;
(RS)-4-(4-fluoro-2-methyl-phenyl)-4,5-dihydro-oxazol-2-ylamine;
(+)-(S)-4-(4-bromo-2-trifluoromethyl-phenyl)-4,5-dihydro-oxazol-2-ylamine;
(+)-(S)-4-(5-chloro-2-trifluoromethyl-phenyl)-4,5-dihydro-oxazol-2-ylamine;
(RS)-4-(4-chloro-2-ethyl-phenyl)-4,5-dihydro-oxazol-2-ylamine;
(+)-(S)-4-(4-fluoro-2-methyl-phenyl)-4,5-dihydro-oxazol-2-ylamine;
(RS)-4-(3-chloro-4-methyl-phenyl)-4,5-dihydro-oxazol-2-ylamine;
(RS)-4-(4-chloro-2-cyclopropyl-phenyl)-4,5-dihydro-oxazol-2-ylamine;
(S)-4-(4-chloro-2-ethyl-phenyl)-4,5-dihydro-oxazol-2-ylamine;
(RS)-4-(3-fluoro-2-methyl-phenyl)-4,5-dihydro-oxazol-2-ylamine;
(S)-4-(4-chloro-2-cyclopropyl-phenyl)-4,5-dihydro-oxazol-2-ylamine;
(RS)-4-(3-fluoro-2-trifluoromethyl-phenyl)-4,5-dihydro-oxazol-2-ylamine;
(S)-4-(3-chloro-4-methyl-phenyl)-4,5-dihydro-oxazol-2-ylamine;
(S)-4-(2-chloro-benzyl)-4,5-dihydro-oxazol-2-ylamine;
(S)-4-(3-trifluoromethyl-benzyl)-4,5-dihydro-oxazol-2-ylamine;
(RS)-4-(2-fluoro-5-methyl-benzyl)-4,5-dihydro-oxazol-2-ylamine;
(R)-4-phenethyl-4,5-dihydro-oxazol-2-ylamine;
(S)-4-phenethyl-4,5-dihydro-oxazol-2-ylamine;
(RS)-4-methyl-4-phenethyl-4,5-dihydro-oxazol-2-ylamine;
(S)-4-methyl-4-phenethyl-4,5-dihydro-oxazol-2-ylamine;
(S)-4-[2-(4-fluoro-phenyl)-ethyl]-4,5-dihydro-oxazol-2-ylamine;
(S)-4-[2-(4-fluoro-3-methoxy-phenyl)-ethyl]-4,5-dihydro-oxazol-2-ylamine;
(S)-4-[2-(2,4-difluoro-phenyl)-ethyl]-4,5-dihydro-oxazol-2-ylamine;
(S)-4-[2-(3,4-difluoro-phenyl)-ethyl]-4,5-dihydro-oxazol-2-ylamine;
(S)-4-[2-(2-trifluoromethyl-phenyl)-ethyl]-4,5-dihydro-oxazol-2-ylamine;
(S)-4-[2-(2-fluoro-phenyl)-ethyl]-4,5-dihydro-oxazol-2-ylamine;
(S)-4-[2-(3-fluoro-phenyl)-ethyl]-4,5-dihydro-oxazol-2-ylamine;
(S)-4-[2-(3,5-difluoro-phenyl)-ethyl]-4,5-dihydro-oxazol-2-ylamine;
(S)-4-(2-o-tolyl-ethyl)-4,5-dihydro-oxazol-2-ylamine;
(S)-4-(2-m-tolyl-ethyl)-4,5-dihydro-oxazol-2-ylamine;
(S)-4-(2-p-tolyl-ethyl)-4,5-dihydro-oxazol-2-ylamine;
(S)-4-[2-(4-trifluoromethyl-phenyl)-ethyl]-4,5-dihydro-oxazol-2-ylamine;
(S)-4-[2-(3-trifluoromethyl-phenyl)-ethyl]-4,5-dihydro-oxazol-2-ylamine;
(R)-4-[2-(3,4-dichloro-phenyl)-ethyl]-4,5-dihydro-oxazol-2-ylamine;
(S)-4-[2-(3,4-dichloro-phenyl)-ethyl]-4,5-dihydro-oxazol-2-ylamine;
(S)-4-[2-(4-chloro-2-fluoro-phenyl)-ethyl]-4,5-dihydro-oxazol-2-ylamine;
(S)-4-[2-(4-fluoro-3-trifluoromethyl-phenyl)-ethyl]-4,5-dihydro-oxazol-2-ylamine;
(S)-4-[2-(3-fluoro-4-trifluoromethyl-phenyl)-ethyl]-4,5-dihydro-oxazol-2-ylamine;
(S)-4-[2-(3,5-dichloro-phenyl)-ethyl]-4,5-dihydro-oxazol-2-ylamine;
(S)-4-[2-(2-chloro-4-fluoro-phenyl)-ethyl]-4,5-dihydro-oxazol-2-ylamine;
(S)-4-[2-(4-fluoro-3-methyl-phenyl)-ethyl]-4,5-dihydro-oxazol-2-ylamine;
(S)-4-{2-[3-(1,1,2,2-tetrafluoro-ethoxy)-phenyl]-ethyl}-4,5-dihydro-oxazol-2-ylamine;
(S)-4-[2-(2-fluoro-3-trifluoromethyl-phenyl)-ethyl]-4,5-dihydro-oxazol-2-ylamine;
(S)-4-[2-(3-bromo-phenyl)-ethyl]-4,5-dihydro-oxazol-2-ylamine;
(S)-4-[2-(4-chloro-3-trifluoromethyl-phenyl)-ethyl]-4,5-dihydro-oxazol-2-ylamine;
(S)-4-[2-(3-fluoro-4-trifluoromethoxy-phenyl)-ethyl]-4,5-dihydro-oxazol-2-ylamine;
(S)-4-[2-(4-fluoro-3-trifluoromethoxy-phenyl)-ethyl]-4,5-dihydro-oxazol-2-ylamine;
(S)-4-[2-(2,3-dichloro-phenyl)-ethyl]-4,5-dihydro-oxazol-2-ylamine;
(S)-4-[2-(3-chloro-4-trifluoromethoxy-phenyl)-ethyl]-4,5-dihydro-oxazol-2-ylamine;
(S)-4-[2-(2-chloro-3-trifluoromethyl-phenyl)-ethyl]-4,5-dihydro-oxazol-2-ylamine;
(S)-4-(1-methyl-2-phenyl-ethyl)-4,5-dihydro-oxazol-2-ylamine;
(S)-4-[2-(2-fluoro-5-trifluoromethyl-phenyl)-ethyl]-4,5-dihydro-oxazol-2-ylamine;
(S)-4-[2-(3-fluoro-5-trifluoromethyl-phenyl)-ethyl]-4,5-dihydro-oxazol-2-ylamine;
(S)-4-[2-(2,5-bis-trifluoromethyl-phenyl)-ethyl]-4,5-dihydro-oxazol-2-ylamine;
(S)-4-[2-(4-chloro-phenyl)-butyl]-4,5-dihydro-oxazol-2-ylamine;
(S)-4-(2-phenyl-butyl)-4,5-dihydro-oxazol-2-ylamine;
(S)-4-[2-(4-trifluoromethyl-phenyl)-propyl]-4,5-dihydro-oxazol-2-ylamine;
(S)-4-[2-(4-trifluoromethyl-phenyl)-butyl]-4,5-dihydro-oxazol-2-ylamine;
(S)-4-[2-(3-trifluoromethyl-phenyl)-propyl]-4,5-dihydro-oxazol-2-ylamine;
(S)-4-[2-(3-trifluoromethyl-phenyl)-butyl]-4,5-dihydro-oxazol-2-ylamine;
(S)-4-[2-(3,5-difluoro-phenyl)-propyl]-4,5-dihydro-oxazol-2-ylamine;
(S)-4-[2-(3,5-difluoro-phenyl)-butyl]-4,5-dihydro-oxazol-2-ylamine;
(S)-4-[2-(4-fluoro-phenyl)-butyl]-4,5-dihydro-oxazol-2-ylamine;
(S)-4-[2-(3,4-difluoro-phenyl)-butyl]-4,5-dihydro-oxazol-2-ylamine;
(S)-4-[2-(2-fluoro-phenyl)-propyl]-4,5-dihydro-oxazol-2-ylamine;
(S)-4-[2-(3,4-dichloro-phenyl)-propyl]-4,5-dihydro-oxazol-2-ylamine;
(S)-4-[2-(3,4-dichloro-phenyl)-butyl]-4,5-dihydro-oxazol-2-ylamine;
(S)-4-[2-(2-fluoro-phenyl)-butyl]-4,5-dihydro-oxazol-2-ylamine;
(S)-4-[2-(2-chloro-phenyl)-ethyl]-4-methyl-4,5-dihydro-oxazol-2-ylamine;
(S)-4-[2-(3-chloro-phenyl)-ethyl]-4,5-dihydro-oxazol-2-ylamine;
(S)-4-[2-(4-chloro-phenyl)-ethyl]-4-methyl-4,5-dihydro-oxazol-2-ylamine;
(S)-4-[2-(3-chloro-phenyl)-ethyl]-4-methyl-4,5-dihydro-oxazol-2-ylamine;
(S)-4-[2-(4-fluoro-phenyl)-ethyl]-4-methyl-4,5-dihydro-oxazol-2-ylamine;
(S)-4-[2-(4-chloro-phenyl)-ethyl]-4,5-dihydro-oxazol-2-ylamine;
(S)-4-[2-(2-chloro-phenyl)-ethyl]-4,5-dihydro-oxazol-2-ylamine;
(S)-4-[2-(3-chloro-phenyl)-butyl]-4,5-dihydro-oxazol-2-ylamine;
(S)-4-((S)-2-phenyl-butyl)-4,5-dihydro-oxazol-2-ylamine;
(S)-4-((R)-2-phenyl-butyl)-4,5-dihydro-oxazol-2-ylamine;
(S)-4-[2-(2-chloro-6-fluoro-phenyl)-ethyl]-4,5-dihydro-oxazol-2-ylamine;
(S)-4-[2-(2,4-dichloro-phenyl)-ethyl]-4-methyl-4,5-dihydro-oxazol-2-ylamine;
(S)-4-[2-(2-bromo-phenyl)-ethyl]-4,5-dihydro-oxazol-2-ylamine;
(S)-4-[2-(2,5-dichloro-phenyl)-ethyl]-4,5-dihydro-oxazol-2-ylamine;
(S)-4-[2-(3-fluoro-phenyl)-butyl]-4,5-dihydro-oxazol-2-ylamine;
(S)-4-[2-(3-chloro-2-fluoro-phenyl)-butyl]-4,5-dihydro-oxazol-2-ylamine;
(S)-4-[2-(3-chloro-4-fluoro-phenyl)-butyl]-4,5-dihydro-oxazol-2-ylamine;
(S)-4-[2-(3-chloro-5-fluoro-phenyl)-butyl]-4,5-dihydro-oxazol-2-ylamine;
(S)-4-[2-(5-chloro-2-fluoro-phenyl)-butyl]-4,5-dihydro-oxazol-2-ylamine;
(S)-4-(4-fluoro-phenoxymethyl)-4,5-dihydro-oxazol-2-ylamine;
(S)-4-(4-chloro-phenoxymethyl)-4,5-dihydro-oxazol-2-ylamine;
(S)-4-(3,4-dichloro-phenoxymethyl)-4,5-dihydro-oxazol-2-ylamine;
(S)-4-(3,5-dichloro-phenoxymethyl)-4,5-dihydro-oxazol-2-ylamine,
(S)-4-(4-bromo-phenoxymethyl)-4,5-dihydro-oxazol-2-ylamine;
(S)-4-(3-chloro-phenoxymethyl)-4,5-dihydro-oxazol-2-ylamine;
(S)-4-(2,4-difluoro-phenoxymethyl)-4,5-dihydro-oxazol-2-ylamine;
(S)-4-(2-fluoro-phenoxymethyl)-4,5-dihydro-oxazol-2-ylamine;
(R)-4-(3-Phenyl-propyl)-4,5-dihydro-oxazol-2-ylamine;
(R)-4-phenylsulfanylmethyl-4,5-dihydro-oxazol-2-ylamine;
(R)-4-benzenesulfonylmethyl-4,5-dihydro-oxazol-2-ylamine;
(R)-4-benzylsulfanylmethyl-4,5-dihydro-oxazol-2-ylamine;
(R)-4-(4-chloro-phenylsulfanylmethyl)-4,5-dihydro-oxazol-2-ylamine;
(S)-4-[1-(4-chloro-phenyl)-cyclopropylmethyl]-4,5-dihydro-oxazol-2-ylamine;
(S)-4-[1-(4-chloro-phenyl)-cyclobutylmethyl]-4,5-dihydro-oxazol-2-ylamine;
(S)-4-(1-phenyl-cyclopropylmethyl)-4,5-dihydro-oxazol-2-ylamine;
(S)-4-[(benzyl-ethyl-amino)-methyl]-4,5-dihydro-oxazol-2-ylamine;
(R)-4-[(dimethyl-phenyl-silanyl)-methyl]-4,5-dihydro-oxazol-2-ylamine;
(RS)-4-(2,3-dihydro-benzo[1,4]dioxin-6-yl)-4,5-dihydro-oxazol-2-ylamine;
(RS)-4-naphthalen-2-yl-4,5-dihydro-oxazol-2-ylamine;
(RS)-4-naphthalen-1-yl-4,5-dihydro-oxazol-2-ylamine;
(R)-4-naphthalen-1-ylmethyl-4,5-dihydro-oxazol-2-ylamine;
(S)-4-naphthalen-1-ylmethyl-4,5-dihydro-oxazol-2-ylamine;
(S)-4-naphthalen-2-ylmethyl-4,5-dihydro-oxazol-2-ylamine;
(RS)-4-(1,2,3,4-tetrahydro-naphthalen-2-yl)-4,5-dihydro-oxazol-2-ylamine;
(S)-4-naphthalen-2-yl-4,5-dihydro-oxazol-2-ylamine;
(S)-4-[2-(3-fluoro-pyridin-4-yl)-ethyl]-4,5-dihydro-oxazol-2-ylamine;
(S)-4-[2-(6-trifluoromethyl-pyridin-2-yl)-ethyl]-4,5-dihydro-oxazol-2-ylamine;
(S)-4-[2-(2-methyl-pyridin-4-yl)-ethyl]-4,5-dihydro-oxazol-2-ylamine;
(S)-4-(2-cyclohexyl-ethyl)-4,5-dihydro-oxazol-2-ylamine;
(S)-4-phenyl-4,5-dihydro-oxazol-2-ylamine;
(R)-4-phenyl-4,5-dihydro-oxazol-2-ylamine;
(S)-4-benzyl-4,5-dihydro-oxazol-2-ylamine;
(RS)-4-phenethyl-4,5-dihydro-oxazol-2-ylamine;
(S)-4-(4-(Naphthalen-1-ylamino)phenethyl)-4,5-dihydrooxazol-2-amine;
(S)-4-(4-(8-Chloronaphthalen-1-ylamino)phenethyl)-4,5-dihydrooxazol-2-amine;
(S)-4-{2-[4-(4-Chloro-phenylamino)-phenyl]-ethyl}-4,5-dihydro-oxazol-2-ylamine;
(S)-4-{2-[4-(4-Chloro-2-fluoro-phenylamino)-phenyl]-ethyl}-4,5-dihydro-oxazol-2-ylamine;
(S)-4-{2-[4-(4-Trifluoromethyl-phenylamino)-phenyl]-ethyl}-4,5-dihydro-oxazol-2-ylamine;
(S)-4-{2-[4-(4-Methoxy-phenylamino)-phenyl]-ethyl}-4,5-dihydro-oxazol-2-ylamine;
(S)-4-(4-(3-Methyl-4-(trifluoromethoxy)phenylamino)phenethyl)-4,5-dihydrooxazol-2-amine;
(S)-4-[2-(4-Phenylamino-phenyl)-ethyl]-4,5-dihydro-oxazol-2-ylamine;
(S)-4-(4-(p-Tolylamino)phenethyl)-4,5-dihydrooxazol-2-amine;
(S)-4-(4-(3,4-Dichlorophenylamino)phenethyl)-4,5-dihydrooxazol-2-amine;
(S)-4-(4-(Quinolin-8-ylamino)phenethyl)-4,5-dihydrooxazol-2-amine;
(S)-4-(4-(5-Fluoropyridin-2-ylamino)phenethyl)-4,5-dihydrooxazol-2-amine;
(S)-4-(4-(6-Methylquinolin-8-ylamino)phenethyl)-4,5-dihydrooxazol-2-amine;
{4-[2-((S)-2-Amino-4,5-dihydro-oxazol-4-yl)-ethyl]-phenyl}-(5-trifluoromethyl-pyridin-2-yl)-amine;
{4-[2-((S)-2-Amino-4,5-dihydro-oxazol-4-yl)-ethyl]-phenyl}-(5-chloro-pyridin-2-yl)-amine;
6-{-4-[2-((S)-2-Amino-4,5-dihydro-oxazol-4-yl)-ethyl]-phenylamino}-nicotinonitrile;
(S)-4-(4-(6-(Trifluoromethyl)pyrimidin-4-ylamino)phenethyl)-4,5-dihydrooxazol-2-amine;
{4-[2-((S)-2-Amino-4,5-dihydro-oxazol-4-yl)-ethyl]-phenyl}-(5-chloro-pyrimidin-2-yl)-amine;
{4-[2-((S)-2-Amino-4,5-dihydro-oxazol-4-yl)-ethyl]-phenyl}-(5-fluoro-pyrimidin-2-yl)-amine;
{4-[2-((S)-2-Amino-4,5-dihydro-oxazol-4-yl)-ethyl]-phenyl}-(3-fluoro-pyridin-2-yl)-amine;
{4-[2-((S)-2-Amino-4,5-dihydro-oxazol-4-yl)-ethyl]-phenyl}-(4-fluoro-pyridin-2-yl)-amine;
{4-[2-((S)-2-Amino-4,5-dihydro-oxazol-4-yl)-ethyl]-phenyl}-(4-trifluoromethyl-pyridin-2-yl)-amine;
{4-[2-((S)-2-Amino-4,5-dihydro-oxazol-4-yl)-ethyl]-phenyl}-(6-methyl-pyrimidin-4-yl)-amine;
{4-[2-((S)-2-Amino-4,5-dihydro-oxazol-4-yl)-ethyl]-phenyl}-(6-methyl-pyridin-2-yl)-amine;
{4-[2-((S)-2-Amino-4,5-dihydro-oxazol-4-yl)-ethyl]-phenyl}-(6-trifluoromethyl-pyridin-2-yl)-amine;
{4-[2-((S)-2-Amino-4,5-dihydro-oxazol-4-yl)-ethyl]-phenyl}-(6-chloro-pyrazin-2-yl)-amine;
{4-[2-((S)-2-Amino-4,5-dihydro-oxazol-4-yl)-ethyl]-phenyl}-(6-chloro-pyridin-2-yl)-amine;
{4-[2-((S)-2-Amino-4,5-dihydro-oxazol-4-yl)-ethyl]-phenyl}-(5-methyl-pyridin-2-yl)-amine;
{4-[2-((S)-2-Amino-4,5-dihydro-oxazol-4-yl)-ethyl]-phenyl}-(5-methoxy-pyridin-2-yl)-amine;
6-{-4-[2-((S)-2-Amino-4,5-dihydro-oxazol-4-yl)-ethyl]-phenylamino}-pyrazine-2-carbonitrile;
{4-[2-((S)-2-Amino-4,5-dihydro-oxazol-4-yl)-ethyl]-phenyl}-(6-chloro-pyrimidin-4-yl)-amine;
{4-[2-((S)-2-Amino-4,5-dihydro-oxazol-4-yl)-ethyl]-phenyl}-(6-methoxy-pyrimidin-4-yl)-amine;
{4-[2-((S)-2-Amino-4,5-dihydro-oxazol-4-yl)-ethyl]-phenyl}-(2-methyl-pyrimidin-4-yl)-amine;
(S)-4-(4-(Pyrimidin-4-ylamino)phenethyl)-4,5-dihydrooxazol-2-amine hydrochloride;
{4-[2-((S)-2-Amino-4,5-dihydro-oxazol-4-yl)-ethyl]-phenyl}-(4-methoxy-pyrimidin-2-yl)-amine;
{4-[2-((S)-2-Amino-4,5-dihydro-oxazol-4-yl)-ethyl]-phenyl}-(5-ethyl-pyrimidin-2-yl)-amine;
{4-[2-((S)-2-Amino-4,5-dihydro-oxazol-4-yl)-ethyl]-phenyl}-(5-methoxy-pyrimidin-2-yl)-amine;
5-{-4-[2-((S)-2-Amino-4,5-dihydro-oxazol-4-yl)-ethyl]-phenylamino}-pyrazine-2-carbonitrile;
{4-[2-((S)-2-Amino-4,5-dihydro-oxazol-4-yl)-ethyl]-phenyl}-(4-trifluoromethyl-pyrimidin-2-yl)-amine;
{4-[2-((S)-2-Amino-4,5-dihydro-oxazol-4-yl)-ethyl]-phenyl}-(3-chloro-pyrazin-2-yl)-amine;
{4-[2-((S)-2-Amino-4,5-dihydro-oxazol-4-yl)-ethyl]-phenyl}-(6-chloro-4-trifluoromethyl-pyridin-2-yl)-amine;
{4-[2-((S)-2-Amino-4,5-dihydro-oxazol-4-yl)-ethyl]-phenyl}-(6-methyl-pyrazin-2-yl)-amine;
{4-[2-((S)-2-Amino-4,5-dihydro-oxazol-4-yl)-ethyl]-phenyl}-(6-chloro-2-methoxy-pyrimidin-4-yl)-amine;
{4-[2-((S)-2-Amino-4,5-dihydro-oxazol-4-yl)-ethyl]-phenyl}-(4-methylsulfanyl-pyrimidin-2-yl)-amine;
{4-[2-((S)-2-Amino-4,5-dihydro-oxazol-4-yl)-ethyl]-phenyl}-(5-methyl-pyrimidin-2-yl)-amine;
1-(2-{4-[2-((S)-2-Amino-4,5-dihydro-oxazol-4-yl)-ethyl]-phenylamino}-pyrimidin-5-yl)-ethanone;
{4-[2-((S)-2-Amino-4,5-dihydro-oxazol-4-yl)-ethyl]-phenyl}-(4-methyl-pyrimidin-2-yl)-amine;
{4-[2-((S)-2-Amino-4,5-dihydro-oxazol-4-yl)-ethyl]-phenyl}-(5-propyl-pyrimidin-2-yl)-amine;
{4-[2-((S)-2-Amino-4,5-dihydro-oxazol-4-yl)-ethyl]-phenyl}-(2-chloro-pyrimidin-5-yl)-amine;
{4-[2-((S)-2-Amino-4,5-dihydro-oxazol-4-yl)-ethyl]-phenyl}-(5-bromo-pyrimidin-2-yl)-amine;
{4-[2-((4S,5S)-2-Amino-5-methyl-4,5-dihydro-oxazol-4-yl)-ethyl]-phenyl}-(5-chloro-pyrimidin-2-yl)-amine;
{4-[2-((S)-2-Amino-4,5-dihydro-oxazol-4-yl)-ethyl]-phenyl}-(5-cyclopropyl-pyrimidin-2-yl)-amine;
{4-[2-((S)-2-Amino-4,5-dihydro-oxazol-4-yl)-ethyl]-phenyl}-(5-ethoxy-pyrimidin-2-yl)-amine;
(S)-4-(4-(5-(Trifluoromethyl)pyrimidin-2-ylamino)phenethyl)-4,5-dihy drooxazol-2-amine;
(S)-4-(4-(5-tert-Butylpyrimidin-2-ylamino)phenethyl)-4,5-dihydrooxazol-2-amine;
(S)-4-(4-(5-(Pentan-3-yl)pyrimidin-2-ylamino)phenethyl)-4,5-dihydrooxazol-2-amine;
2-{-4-[2-((S)-2-Amino-4,5-dihydro-oxazol-4-yl)-ethyl]-phenylamino}-pyrimidine-5-carbonitrile;
(S)-4-(4-(5-Cyclobutylpyrimidin-2-ylamino)phenethyl)-4,5-dihydrooxazol-2-amine;
(S)-4-(4-(5-Isopropylpyrimidin-2-ylamino)phenethyl)-4,5-dihydrooxazol-2-amine;
{4-[2-((S)-2-Amino-4,5-dihydro-oxazol-4-yl)-ethyl]-2-methyl-phenyl}-(5-chloro-pyrimidin-2-yl)-amine; and
(S)-4-(4-(5-(2,2,2-Trifluoroethoxy)pyrimidin-2-ylamino)phenethyl)-4,5-dihydrooxazol-2-amine;
   or a pharmaceutically acceptable salt, a racemic mixture, an enantiomer and/or optical isomer (stereoisomer), a tautomer, a prodrug, or a solvate thereof.

The compounds described herein can be prepared according to methods known in the art, for example, the methods as described in U.S. Pat. Nos. 7,902,238 B2, 8,354,441 B2, 8,604,061 B2, 8,673,950 B2, and 9,132,136 B2; U.S. Patent Application Publication Nos. 2010/0311798 A1, 2013/0345201 A1, 2013/0345241 A1, 2015/0045359 A1; and WO 2008/092785 A1, WO 2008/098857 A1, and WO 2012/016879 A1. These patent documents are herein incorporated in their entireties.

### IV. Imidazole Compounds

In another aspect, the present disclosure provides method of treating pain in a subject, comprising administering to a subject in need of such treatment a therapeutically effective amount of a compound of Formulae (IIa) or (IIb):

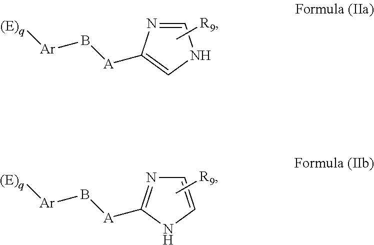

or a pharmaceutically acceptable salt, a racemic mixture, an enantiomer and/or optical isomer (stereoisomer), a tautomer, a prodrug, or a solvate thereof,

wherein: —A—B— is —CH(R₈)—, —N(R)—CH(R₈)—, —CH(R₈)—N(R_{T})—, —NH—NH—, —O—CH(R₈)—, —CH(R₈)—O—, —S—CH(R₈), —CH(R₈)—S—, or —CH(R₈)—CH(R₈)—;

R₇ and R₈ are each independently hydrogen, lower alkyl, lower alkenyl, cycloalkyl, lower alkyl substituted by hydroxy or halogen, —(CH₂)_{d}—S-lower alkyl, —(CH₂)_{d}—O-lower alkyl, —(CH₂)_{d}—NHC(O)O-lower alkyl, —(CH₂)_{d}-aryl, or —(CH₂)_{d}-heteroaryl;

R₉ is hydrogen, halogen, lower alkyl, or amino;

Ar is phenyl, naphthyl, benzofuranyl, benzo[1,3]dioxolyl, pyrimidin-2-yl, pyrimidin-4-yl, or pyridin-3-yl,

E is hydrogen, halogen, lower alkyl optionally substituted by one or more halogens, lower alkoxy optionally substituted by one or more halogens, —(CH₂)ₚ-aryl, —(CH₂)ₚ-heteroaryl, —O—(CH₂)ₚ-aryl, —O—(CH₂)ₚ-heteroaryl, —(CH₂)ᵣ-phenyl optionally substituted by lower alkoxy, —(CH₂)ᵣ—C(O)-phenyl optionally substituted by lower alkoxy, —(CH₂)ᵣ—O-phenyl optionally substituted by lower alkoxy, cycloalkyl, morpholinyl, NO₂, amino, hydroxy, —CH(OH)-phenyl, or —NHC(O)aryl;

p is 0 or 1;

q is 0, 1, 2, 3, or 4;

r is 0, 1, 2, 3; and

d is 0, 1, 2, or 3;

and

wherein the compound is a TAAR1 agonist or partial agonist.

In one embodiment, R₉ is hydrogen.

In one embodiment, —A—B— is-CH₂— or —CH₂—N(R₇)—, wherein R₇ is hydrogen, lower alkyl, cycloalkyl, phenyl, or benzyl.

In one embodiment, Ar is phenyl.

In one embodiment, E is hydrogen, halogen, hydroxyl, amino, C₁-C₇ alkyl optionally substituted by one or more halogens, C₁-C₇ alkoxy optionally substituted by one or more halogens, phenyl, benzyl, pyridyl, pyrrolyl, phenyloxy, benzyloxy, naphthyl, —CHOH-phenyl, or —O-[3-pyrindyl].

In one embodiment, R₉ is hydrogen; —A—B— is —CH₂— or —CH₂—N(R₇)—, wherein R₇ is hydrogen, methyl, ethyl, propyl, isopropyl, cyclopropyl, or benzyl; Ar is phenyl; and E is hydrogen, halogen, C₁-C₄ alkyl optionally substituted by one to five halogens, C₁-C₄ alkyloxy optionally substituted by one to five halogens, phenyl, or benzyl.

In one embodiment, the compound of Formula (IIa) is N-((1H-imidazol-4-yl)methyl)-4-chloro-N-isopropylaniline (compound 1), having the following structure:

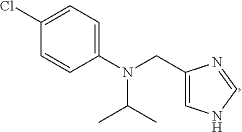 [SMILES: CC(C)N(Cc1c[nH]cn1)c1ccc(Cl)cc1]

or a pharmaceutically acceptable salt, a racemic mixture, an enantiomer and/or optical isomer (stereoisomer), a tautomer, a prodrug, or a solvate thereof.

In one embodiment, the compound of Formula (IIb) is 2-(2,6-diethylbenzyl)-1H-imidazole, having the following structure:

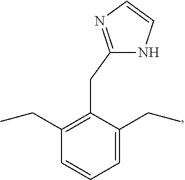 [SMILES: CCc1cccc(CC)c1Cc1ncc[nH]1]

or a pharmaceutically acceptable salt, a racemic mixture, an enantiomer and/or optical isomer (stereoisomer), a tautomer, a prodrug, or a solvate thereof.

Compounds of Formulae (IIa) and (IIb) also include the following compounds:
(3-chloro-phenyl)-(1H-imidazol-2-ylmethyl)-methyl-amine;
(4-chloro-phenyl)-(1H-imidazol-2-ylmethyl)-methyl-amine;
(1H-imidazol-2-ylmethyl)-(3-methoxy-phenyl)-methyl-amine;
(4-fluoro-phenyl)-(1H-imidazol-2-ylmethyl)-isopropyl-amine;
ethyl-(3-fluoro-phenyl)-(1H-imidazol-2-ylmethyl)-amine;
(3-chloro-phenyl)-(1H-imidazol-2-ylmethyl)-isopropyl-amine;
(3-chloro-phenyl)-ethyl-(1H-imidazol-2-ylmethyl)-amine;
(2,5-difluoro-phenyl)-(3H-imidazol-4-ylmethyl)-isopropyl-amine;
(1H-imidazol-2-ylmethyl)-isopropyl-m-tolyl-amine;
(3-benzyloxy-phenyl)-(1H-imidazol-2-ylmethyl)-isopropyl-amine;
(1H-imidazol-2-ylmethyl)-isopropyl-[3-(pyridin-3-yloxy)-phenyl]-amine;
(3-benzyl-phenyl)-(1H-imidazol-2-ylmethyl)-isopropyl-amine;
biphenyl-3-yl-(1H-imidazol-2-ylmethyl)-isopropyl-amine;
[3-(4-chloro-phenoxy)-phenyl]-(1H-imidazol-2-ylmethyl)-isopropyl-amine;
Ethyl-(1H-imidazol-2-ylmethyl)-(3-phenoxy-phenyl)-amine;
(3-benzyloxy-phenyl)-ethyl-(1H-imidazol-2-ylmethyl)-amine;
(3,4-dichloro-phenyl)-ethyl-(1H-imidazol-2-ylmethyl)-amine;
(1H-imidazol-2-ylmethyl)-(2-methoxy-ethyl)-phenyl-amine;
(1H-imidazol-2-ylmethyl)-(3-phenoxy-phenyl)-amine;
(3-benzyloxy-phenyl)-(1H-imidazol-2-ylmethyl)-amine;
(4-chloro-3-methoxy-phenyl)-(1H-imidazol-2-ylmethyl)-amine;
(3-bromo-phenyl)-(1H-imidazol-2-ylmethyl)-amine;
(3,4-difluoro-phenyl)-(1H-imidazol-2-ylmethyl)-amine;
(3-chloro-4-fluoro-phenyl)-(1H-imidazol-2-ylmethyl)-amine;
(3,4-dichloro-phenyl)-(1H-imidazol-2-ylmethyl)-amine;
(4-chloro-phenyl)-(4,5-dihydro-1H-imidazol-2-ylmethyl)-amine;
(3-chloro-phenyl)-(4,5-dihydro-1H-imidazol-2-ylmethyl)-amine;
(4-bromo-3-chloro-phenyl)-(4,5-dihydro-1H-imidazol-2-ylmethyl)-amine;
(4-chloro-3-fluoro-phenyl)-(4,5-dihydro-1H-imidazol-2-ylmethyl)-amine;
(3-chloro-phenyl)-(4,5-dihydro-1H-imidazol-2-ylmethyl)-methyl-amine;
(3,4-dichloro-phenyl)-(4,5-dihydro-1H-imidazol-2-ylmethyl)-methyl-amine;
(4,5-dihydro-1H-imidazol-2-ylmethyl)-[4-(4-methoxy-benzyl)-phenyl]-amine;
(4,5-dihydro-1H-imidazol-2-ylmethyl)-(2-methoxy-phenyl)-methyl-amine;
2-(2-chloro-phenoxymethyl)-4,5-dihydro-1H-imidazole;
2-(2,3-dichloro-phenoxymethyl)-4,5-dihydro-1H-imidazole;
2-(3-trifluoromethyl-phenoxymethyl)-4,5-dihydro-1H-imidazole;
2-[1-(2,6-dichloro-phenoxy)-ethyl]-4,5-dihydro-1H-imidazole;
3-{4-[1-(4,5-dihydro-1H-imidazol-2-yl)-ethoxy]-phenyl}-1-(4-isopropoxy-phenyl)-propan-1-one;
2-[4-(4-isopropoxy-phenoxymethyl)-phenoxymethyl]-4,5-dihydro-1H-imidazole;
2-(3-benzyl-phenoxymethyl)-4,5-dihydro-1H-imidazole;
2-[4-(3-phenyl-propyl)-phenoxymethyl]-4,5-dihydro-1H-imidazole;
2-(2-chloro-3-trifluoromethyl-phenoxymethyl)-4,5-dihydro-1H-imidazole;
2-(2,3-difluoro-phenoxymethyl)-4,5-dihydro-1H-imidazole;
4-(4,5-dihydro-1H-imidazol-2-ylmethoxy)-2,3-dimethyl-phenol;
2-(3,4-dichloro-phenoxymethyl)-4,5-dihydro-1H-imidazole;
N-(3-chloro-2-methyl-phenyl)-N′-(4,5-dihydro-1H-imidazol-2-yl)-hydrazine;
N-(4,5-dihydro-1H-imidazol-2-yl)-N′-(3,4-dimethyl-phenyl)-hydrazine;
2-(2,3-dichloro-phenylsulfanylmethyl)-4,5-dihydro-1H-imidazole;
(4,5-dihydro-1H-imidazol-2-ylmethyl)-(4-methyl-benzofuran-5-yl)-amine;
2-(benzofuran-6-yloxymethyl)-4,5-dihydro-1H-imidazole;
2-[2-(3-chloro-phenyl)-2-methoxy-ethyl]-1H-imidazole;
(R,S)-(1H-imidazol-2-yl)-(1-phenyl-ethyl)-amine;
(R,S)-(1H-imidazol-2-yl)-(1-phenyl-propyl)-amine;
(R,S)-(1H-imidazol-2-yl)-(1-o-tolyl-ethyl)-amine;
(R,S)-[1-(2,3-dichloro-phenyl)-ethyl]-(1H-imidazol-2-yl)-amine;
(R,S)-[1-(2-chloro-phenyl)-ethyl]-(1H-imidazol-2-yl)-amine;
2-(2,3-difluoro-phenoxymethyl)-1H-imidazole;
2-(2,3-dichloro-phenylsulfanylmethyl)-1H-imidazole;
2-(2,3-dichloro-benzenesulfonylmethyl)-1H-imidazole;
4-[2-(2-chloro-phenyl)-ethyl]-1H-imidazole;
4-[2-(2-methoxy-phenyl)-ethyl]-1H-imidazole;
4-[2-(3-chloro-phenyl)-ethyl]-1H-imidazole;
4-[2-(3-fluoro-phenyl)-ethyl]-1H-imidazole;
4-[2-(3-trifluoromethyl-phenyl)-ethyl]-1H-imidazole;
4-[2-(3-methoxy-phenyl)-ethyl]-1H-imidazole;
4-[2-(4-chloro-phenyl)-ethyl]-1H-imidazole;
4-[2-(3,5-dichloro-phenyl)-ethyl]-1H-imidazole;
4-(2-phenyl-butyl)-1H-imidazole;
4-(2,3-dichloro-phenoxymethyl)-1H-imidazole;
4-(2,3-difluoro-phenoxymethyl)-1H-imidazole;
4-(3,4-dichloro-phenoxymethyl)-1H-imidazole;
4-(4-chloro-3-fluoro-phenoxymethyl)-1H-imidazole;
5-(benzofuran-6-yloxymethyl)-1H-imidazole;
5-(2,3-dichloro-phenylsulfanylmethyl)-1-imidazole;
4-(4-chloro-phenylsulfanylmethyl)-5-methyl-1H-imidazole; and
4-(naphthalen-2-ylsulfanylmethyl)-1H-imidazole;
   or a pharmaceutically acceptable salt, a racemic mixture, an enantiomer and/or optical isomer (stereoisomer), a tautomer, a prodrug, or a solvate thereof.

The compounds described herein can be prepared according to methods known in the art, for example, the methods as described in U.S. Pat. Nos. 7,652,055 B2, 7,812,047 B2, 7,834,044 B2, 7,858,652 B2, 7,858,653 B2, and 8,399,463 B2; and U.S. Patent Application Publication Nos. 2008/0096906 A1 and 2015/0045359 A1. These patent documents are incorporated herein in their entireties.

### V. Additional Useful Compounds in the Method of Treating or Preventing Pain

In another aspect, the present disclosure provides a method of treating pain in a subject, comprising administering to a subject in need of such treatment a therapeutically effective amount of a compound of Formula (III):

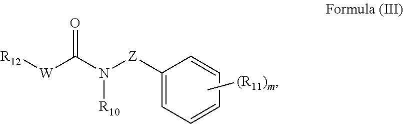

or a pharmaceutically acceptable salt, a racemic mixture, an enantiomer and/or optical isomer (stereoisomer), a tautomer, a prodrug, or a solvate thereof,

wherein:

R₁₀ is hydrogen or lower alkyl;

m is 1 or 2;

R₁₁ independently is —(CH₂)ₖ—(O)ⱼheterocycloalkyl, or —C(O)— heterocycloalkyl, wherein the heterocycloalkyl group is optionally substituted by lower alkyl, hydroxy, halogen, or —(CH₂)_{b}-aryl; or two R₁₁, together with the ring atoms they are attached to, forms heterocycloalkyl optionally substituted by amino;

k is 0, 1, or 2;

j is 0 or 1;

b is 0, 1, or 2;

R₁₂ is (i) lower alkyl, optionally substituted by one or more (same or different) halogens, or cycloalkyl optionally substituted by lower alkoxy or halogen; orindan-2-yl; or

(ii) heterocycloalkyl, optionally substituted by heteroaryl; or

(iii) aryl or heteroaryl, wherein the aromatic rings in aryl and heteroaryl are optionally substituted by one or two substituents independently selected from the group of lower alkyl, halogen, heteroaryl, hydroxy, CF₃, OCF₃, OCH₂CF₃, OCH₂-cycloalkyl, OCH₂C(CH₂OH)(CH₂Cl)(CH₃), S-lower alkyl, lower alkoxy, CH₂-lower alkoxy, lower alkynyl, cyano, —C(O)-phenyl, —O-phenyl, —O—CH₂-phenyl, phenyl, and —CH₂-phenyl, and wherein the phenyl rings are optionally substituted by halogen, —C(O)-lower alkyl, —C(O)OH, or —C(O)O-lower alkyl, or the aromatic rings are optionally substituted by heterocycloalkyl, OCH₂-oxetan-3-yl, or O-tetrahydropyran-4-yl, optionally substituted by lower alkyl;

W is a bond, —N(R₁₃)—, —CH₂NH—, —CH(R₁₄)—, —(CHR₁₄)ᵥ—O—, —O—(CHR₁₄)ᵥ, or —(CH₂)₂—;

Z is a bond or —CH₂—;

R₁₃ is hydrogen or lower alkyl;

R₁₄ is hydrogen, lower alkyl, optionally substituted by one or more (same or different) halogens, or lower alkoxy; and

v is 0, 1,2 or 3.

In another aspect, the present disclosure provides a method of treating pain in a subject, comprising administering to a subject in need of such treatment a therapeutically effective amount of a compound of Formula (IIIa):

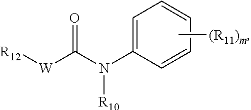

or a pharmaceutically acceptable salt, a racemic mixture, an enantiomer and/or optical isomer (stereoisomer), a tautomer, a prodrug, or a solvate thereof,

wherein:

R₁₀ is hydrogen or lower alkyl;

m is 1 or 2;

R₁₁ each independently is —(CH₂)ₖ—(O)ⱼ-heterocycloalkyl optionally substituted by lower alkyl, hydroxy, halogen, or —(CH₂)_{b}-aryl; or two R₁₁, together with the ring atoms they are attached to, forms heterocycloalkyl optionally substituted by amino;
k is 0, 1, or 2;
j is 0 or 1;
b is 0, 1, or 2;

R₁₂ is cycloalkyl, heterocycloalkyl, aryl, or heteroaryl, wherein the aromatic rings within the aryl or heteroaryl group are optionally substituted by one or two substituents independently selected from the group of lower alkyl, halogen, heteroaryl, CF₃, —OCF₃, —OCH₂CF₃, lower alkoxy, —CH₂-lower alkoxy, lower alkynyl, and cyano;

W is a bond, —N(R₁₃)—, —CH₂NH—, —CH(R₁₄)—, —(CH₂X—O—, or

—(CH₂)₂—;

R₁₃ is hydrogen or lower alkyl;

R₁₄ is hydrogen, lower alkyl, or lower alkoxy; and

v is 0, 1, or 2.

In one embodiment in the compound of Formula (III) or Formula (IIIa), R₁₂ is aryl or heteroaryl, wherein the aromatic rings are optionally substituted by one or two substituents, selected from lower alkyl, halogen, heteroaryl, CF₃, OCF₃, OCH₂CF₃, lower alkoxy, CH₂-lower alkoxy, lower alkynyl, or cyano.

In one embodiment in the compound of Formula (III) or Formula (IIIa), W is a bond or —NR₁₃—.

In one embodiment in the compound of Formula (III) or Formula (IIIa), R₁₂ is aryl or heteroaryl, wherein the aromatic rings are optionally substituted by one or two substituents, selected from lower alkyl, halogen, heteroaryl, CF₃, OCF₃, OCH₂CF₃, lower alkoxy, CH₂-lower alkoxy, lower alkynyl, or cyano; and W is a bond or —NR₁₃—.

In one embodiment in the compound of Formula (III) or Formula (IIIa), R₁₂ is phenyl or pyridinyl, wherein the aromatic rings in the phenyl or pyridinyl group are optionally substituted by one or two substituents independently selected from the group of lower alkyl, halogen, heteroaryl, CF₃, OCF₃, OCH₂CF₃, lower alkoxy, —CH₂-lower alkoxy, lower alkynyl, and cyano.

In one embodiment in the compound of Formula (III) or Formula (IIIa), R₁₂ is phenyl or pyridinyl, wherein the aromatic rings in the phenyl or pyridinyl group are optionally substituted by one or two substituents independently selected from the group of lower alkyl, halogen, heteroaryl, CF₃, OCF₃, OCH₂CF₃, lower alkoxy, —CH₂-lower alkoxy, lower alkynyl, and cyano; and W is a bond or —N(R₁₃)—.

In one embodiment in the compound of Formula (III) or Formula (IIIa), two R₁₁, together with the ring atoms they are attached to, forms heterocycloalkyl optionally substituted by amino.

Suitable compounds of Formula (III) or Formula (IIIa) also include the following compounds:
(RS)-1-(4-Butyl-2-methyl-phenyl)-3-(4-pyrrolidin-3-yl-phenyl)-urea;
1-(3,4-Dichloro-phenyl)-3-[4-(4-methyl-piperazin-1-yl)-phenyl]-urea;
(RS)-1-(3,4-Dichloro-phenyl)-3-(4-pyrrolidin-3-yl-phenyl)-urea;
(RS)-1-(4-Chloro-phenyl)-3-(4-pyrrolidin-3-yl-phenyl)-urea;
(RS)-1-Phenyl-3-(4-pyrrolidin-3-yl-phenyl)-urea;
(RS)-1-(2,4-Dichloro-phenyl)-3-(4-pyrrolidin-3-yl-phenyl)-urea;
(RS)-1-(3-Chloro-phenyl)-3-(4-pyrrolidin-3-yl-phenyl)-urea;
(RS)-1-(4-Pyrrolidin-3-yl-phenyl)-3-(4-trifluoromethyl-phenyl)-urea;
(RS)-1-(5-Chloro-pyridin-2-yl)-3-(4-pyrrolidin-3-yl-phenyl)-urea;
(RS)-1-(6-Chloro-pyridin-3-yl)-3-(4-piperidin-3-yl-phenyl)-urea;
(RS)-1-(5-Chloro-pyridin-2-yl)-3-(4-piperidin-3-yl-phenyl)-urea;
(RS)-1-(5-Chloro-pyridin-2-yl)-3-[4-(2-pyrrolidin-3-yl-ethyl)-phenyl]-urea;
(RS)-1-(4-Chloro-phenyl)-3-[4-(2-pyrrolidin-3-yl-ethyl)-phenyl]-urea;
(RS)-1-(4-Chloro-phenyl)-3-[4-(2-piperidin-3-yl-ethyl)-phenyl]-urea;
(RS)-1-(4-(Morpholin-2-yl)phenyl)-3-(4-(trifluoromethyl)phenyl)urea;
(RS)-1-(4-Chlorophenyl)-3-(4-(morpholin-2-yl)phenyl)urea;
(RS)-1-(4-(Morpholin-2-yl)phenyl)-3-p-tolylurea;
(RS)-1-(6-Chloropyridin-3-yl)-3-(4-(morpholin-2-yl)phenyl)urea;
(RS)-1-(3-Chlorophenyl)-3-(4-(morpholin-2-yl)phenyl)urea;
(RS)-1-(4-(Morpholin-2-yl)phenyl)-3-m-tolylurea;
(RS)-1-(2-Chlorophenyl)-3-(4-(morpholin-2-yl)phenyl)urea;
(RS)-1-(4-Methylbenzyl)-3-(4-(morpholin-2-yl)phenyl)urea;
(R)-1-(4-(Morpholin-2-yl)phenyl)-3-(4-(trifluoromethyl)phenyl)urea;
(S)-1-(4-(Morpholin-2-yl)phenyl)-3-(4-(trifluoromethyl)phenyl)urea;
(RS)—N-(4-Pyrrolidin-3-yl-phenyl)-benzamide;
(RS)-4-Chloro-N-(4-pyrrolidin-3-yl-phenyl)-benzamide;
(RS)—N-(4-Pyrrolidin-3-yl-phenyl)-4-trifluoromethyl-benzamide;
(RS)-2,4-Dichloro-N-(4-pyrrolidin-3-yl-phenyl)-benzamide;
(RS)-3-Chloro-N-(4-pyrrolidin-3-yl-phenyl)-benzamide;
(RS)-4-Chloro-N-[4-(1-methyl-pyrrolidin-3-yl)-phenyl]-benzamide;
(RS)-4-Chloro-N-[4-(1-ethyl-pyrrolidin-3-yl)-phenyl]-benzamide;
(RS)-4-Chloro-N-[4-(pyrrolidin-3-yloxy)-phenyl]-benzamide;
(RS)-5-Chloro-pyridine-2-carboxylic acid (4-pyrrolidin-3-yl-phenyl)-amide;
(RS)-6-Chloro-pyridine-3-carboxylic acid (4-pyrrolidin-3-yl-phenyl)-amide;
(RS)-4-Ethoxy-N-(4-pyrrolidin-3-yl-phenyl)-benzamide;
(RS)-4-Propyl-N-(4-pyrrolidin-3-yl-phenyl)-benzamide;
(RS)-4-Ethynyl-N-(4-pyrrolidin-3-yl-phenyl)-benzamide;
(RS)-4-Cyano-N-(4-pyrrolidin-3-yl-phenyl)-benzamide;
(RS)-3,4-Dichloro-N-(4-pyrrolidin-3-yl-phenyl)-benzamide;
4-Chloro-N-(4-piperidin-4-yl-phenyl)-benzamide;
(RS)-4-Chloro-N-(4-piperidin-3-yl-phenyl)-benzamide;
4-Chloro-N-(4-piperazin-1-yl-phenyl)-benzamide;
4-Chloro-N-[4-((3RS,4RS)-4-fluoro-pyrrolidin-3-yl)-phenyl]-benzamide;
(RS)-4-Chloro-N-[3-(pyrrolidin-3-yloxy)-phenyl]-benzamide;
(RS)-6-Pyrazol-1-yl-N-(4-pyrrolidin-3-yl-phenyl)-nicotinamide;
(RS)-6-Chloro-N-(4-piperidin-3-yl-phenyl)-nicotinamide;
(RS)-4-Chloro-2-fluoro-N-(4-pyrrolidin-3-yl-phenyl)-benzamide;
(RS)-5-Chloro-pyridine-2-carboxylic acid (4-piperidin-3-yl-phenyl)-amide;
(RS)-4-Chloro-N-[4-(4-methyl-morpholin-2-yl)-phenyl]-benzamide;
(RS)-Quinoline-2-carboxylic acid (4-pyrrolidin-3-yl-phenyl)-amide;
(RS)-Isoquinoline-1-carboxylic acid (4-pyrrolidin-3-yl-phenyl)-amide;
(RS)-4-Chloro-pyridine-2-carboxylic acid (4-pyrrolidin-3-yl-phenyl)-amide;
(RS)-5-Bromo-pyridine-2-carboxylic acid (4-pyrrolidin-3-yl-phenyl)-amide;
(RS)-2-Fluoro-4-methoxy-N-(4-pyrrolidin-3-yl-phenyl)-benzamide;
(RS)—N-(4-Pyrrolidin-3-yl-phenyl)-6-(2,2,2-trifluoro-ethoxy)-nicotinamide;
(RS)-6-Methoxy-quinoline-2-carboxylic acid (4-pyrrolidin-3-yl-phenyl)-amide;
(RS)-3-Chloro-N-(4-piperidin-3-yl-phenyl)-benzamide;
(RS)-3,4-Dichloro-N-(4-piperidin-3-yl-phenyl)-benzamide;
(RS)-4-Ethoxy-N-(4-piperidin-3-yl-phenyl)-benzamide;
(RS)—N-(4-Piperidin-3-yl-phenyl)-4-trifluoromethyl-benzamide;
(RS)-4-Chloro-2-fluoro-N-(4-piperidin-3-yl-phenyl)-benzamide;
(RS)-4-Chloro-N-(4-pyrrolidin-2-ylmethyl-phenyl)-benzamide;
(RS)-1-Chloro-isoquinoline-3-carboxylic acid (4-pyrrolidin-3-yl-phenyl)-amide;
(RS)-4-Chloro-N-[4-(2-pyrrolidin-3-yl-ethyl)-phenyl]-benzamide;
(RS)-4-Chloro-N-[4-(2-piperidin-3-yl-ethyl)-phenyl]-benzamide;
4-Chloro-N—((R)-4-piperidin-3-yl-phenyl)-benzamide;
(RS)-5-Chloro-pyridine-2-carboxylic acid [4-(2-pyrrolidin-3-yl-ethyl)-phenyl]-amide;
(RS)—N-(4-Piperidin-3-yl-phenyl)-4-propyl-benzamide;
(RS)-5-Trifluoromethyl-pyridine-2-carboxylic acid (4-piperidin-3-yl-phenyl)-amide;
(RS)-5-Trifluoromethyl-pyridine-2-carboxylic acid (4-pyrrolidin-3-yl-phenyl)-amide;
(RS)-5-Trifluoromethyl-pyridine-2-carboxylic acid (4-morpholin-2-yl-phenyl)-amide;
(RS)-4-Chloro-N-[4-(2-pyrrolidin-2-yl-ethyl)-phenyl]-benzamide;
4-Chloro-N—((R)-4-morpholin-2-yl-phenyl)-benzamide;
4-Chloro-N—((S)-4-morpholin-2-yl-phenyl)-benzamide;
(RS)-4-Chloro-N-[4-(pyrrolidin-3-yloxymethyl)-phenyl]-benzamide;
(RS)-4-Chloro-2-fluoro-N-(4-morpholin-2-yl-phenyl)-benzamide;
(RS)-3,4-Dichloro-N-(4-morpholin-2-yl-phenyl)-benzamide;
(RS)-5-Chloro-pyridine-2-carboxylic acid (4-morpholin-2-yl-phenyl)-amide;
(RS)-4-Chloro-N-(4-pyrrolidin-3-ylmethyl-phenyl)-benzamide;
3,4-Dichloro-N—(R)-(4-piperidin-3-yl-phenyl)-benzamide;
(R)-3-Chloro-N-(4-(piperidin-3-yl)phenyl)benzamide;
3,4-Dichloro-N—((S)-4-piperidin-3-yl-phenyl)-benzamide;
(S)-3-Chloro-N-(4-(piperidin-3-yl)phenyl)benzamide;
(RS)-3,4-Dichloro-N-[4-(2-pyrrolidin-2-yl-ethyl)-phenyl]-benzamide;
(RS)—N-[4-(2-Pyrrolidin-2-yl-ethyl)-phenyl]-4-trifluoromethyl-benzamide;
(RS)-4-Fluoro-N-[4-(2-pyrrolidin-2-yl-ethyl)-phenyl]-benzamide;
(RS)-3-Chloro-N-[4-(2-pyrrolidin-2-yl-ethyl)-phenyl]-benzamide;
(RS)-4-Ethoxy-N-[4-(2-pyrrolidin-2-yl-ethyl)-phenyl]-benzamide;
(RS)-5-Chloro-pyrazine-2-carboxylic acid (4-piperidin-3-yl-phenyl)-amide;
(S)-6-Chloro-N-(4-(piperidin-3-yl)phenyl)nicotinamide;
(R)-5-Chloro-N-(4-(piperidin-3-yl)phenyl)picolinamide;
(S)-5-Chloro-N-(4-(piperidin-3-yl)phenyl)picolinamide;
(RS)-4-Chloro-N-(4-(2-(piperidin-2-yl)ethyl)phenyl)benzamide;
(RS)-5-Ethoxy-N-(4-(2-(pyrrolidin-2-yl)ethyl)phenyl)picolinamide;
(RS)—N-(4-(2-(Piperidin-2-yl)ethyl)phenyl)-4-(trifluoromethyl)benzamide;
(RS)-3,4-Dichloro-N-(4-(2-(piperidin-2-yl)ethyl)phenyl)benzamide;
(RS)-4-Ethynyl-N-(4-(2-(piperidin-2-yl)ethyl)phenyl)benzamide;
(RS)—N-(4-(Piperidin-3-yl)phenyl)-6-(2,2,2-trifluoroethoxy)nicotinamide;
(RS)-5-Ethoxy-pyridine-2-carboxylic acid (4-piperidin-3-yl-phenyl)-amide;
(RS)-4-Methyl-N-(4-(pyrrolidin-3-yl)phenyl)benzamide;
(RS)-4-Methyl-N-(4-(piperidin-3-yl)phenyl)benzamide;
(RS)-4-Methoxy-N-(4-(piperidin-3-yl)phenyl)benzamide;
(RS)—N-(4-(Morpholin-2-yl)phenyl)benzamide;
(RS)-4-Methyl-N-(4-(morpholin-2-yl)phenyl)benzamide;
(RS)-4-Methoxy-N-(4-(morpholin-2-yl)phenyl)benzamide;
(RS)—N-(4-(Piperidin-3-yl)phenyl)-5-(2,2,2-trifluoroethoxy)picolinamide;
(RS)-4-(benzyloxy)-N-(4-(morpholin-2-yl)phenyl)benzamide;
(RS)-6-chloro-N-(4-(morpholin-2-yl)phenyl)nicotinamide;
(RS)-2-(4-(6-cyanonicotinamido)phenyl)morpholin-4-ium chloride;
(R)-4-Methyl-N-(4-(morpholin-2-yl)phenyl)benzamide;
(S)-4-Methyl-N-(4-(morpholin-2-yl)phenyl)benzamide;
(RS)-4-Ethoxy-N-(4-(morpholin-2-yl)phenyl)benzamide;
(RS)-4-Ethyl-N-(4-(morpholin-2-yl)phenyl)benzamide;
(R)-4-Chloro-3-methoxy-N-(4-(piperidin-3-yl)phenyl)benzamide;
(S)-4-Chloro-3-methoxy-N-(4-(piperidin-3-yl)phenyl)benzamide;
(R)-3-Chloro-4-methoxy-N-(4-(piperidin-3-yl)phenyl)benzamide;
(S)-3-Chloro-4-methoxy-N-(4-(piperidin-3-yl)phenyl)benzamide;
(R)—N-(4-(Pyrrolidin-3-yl)phenyl)-6-(2,2,2-trifluoroethoxy)nicotinamide;
(S)—N-(4-(Pyrrolidin-3-yl)phenyl)-6-(2,2,2-trifluoroethoxy)nicotinamide;
(RS)-4-(4-Chlorophenoxy)-N-(4-(morpholin-2-yl)phenyl)benzamide;
(R)-6-Chloro-N-(4-(morpholin-2-yl)phenyl)nicotinamide;
(S)-6-Chloro-N-(4-(morpholin-2-yl)phenyl)nicotinamide;
(RS)-3-chloro-N-(4-(morpholin-2-yl)phenyl)benzamide;
(RS)-5-chloro-N-(4-(morpholin-2-yl)phenyl)nicotinamide;
(RS)-Methyl 4-((4-(4-(morpholin-2-yl)phenylcarbamoyl)phenoxy)methyl)benzoate;
(RS)-Methyl 2-chloro-4-(4-(4-(morpholin-2-yl)phenylcarbamoyl)phenoxy)benzoate;
(RS)-4-Cyclopropylmethoxy-N-(4-morpholin-2-yl-phenyl)-benzamide;
(RS)-4-(Methylthio)-N-(4-(morpholin-2-yl)phenyl)benzamide;
(RS)-2-Chloro-N-(4-(morpholin-2-yl)phenyl)isonicotinamide;
(RS)-5,6-Dichloro-N-(4-(morpholin-2-yl)phenyl)nicotinamide;
(RS)-4-(2-Chloromethyl-3-hydroxy-2-methyl-propoxy)-N-(4-morpholin-2-yl-phenyl)-benzamide;
(RS)-2,6-Dichloro-N-(4-(morpholin-2-yl)phenyl)isonicotinamide;
(RS)—N-(4-(Morpholin-2-yl)phenyl)-6-(2,2,2-trifluoroethoxy)nicotinamide;
(R)—N-(4-(Piperidin-3-yl)phenyl)-6-(2,2,2-trifluoroethoxy)nicotinamide;
(S)—N-(4-(Piperidin-3-yl)phenyl)-6-(2,2,2-trifluoroethoxy)nicotinamide;
(S)-3-Chloro-4-methyl-N-(4-(piperidin-3-yl)phenyl)benzamide;
(S)-4-Chloro-3-methyl-N-(4-(piperidin-3-yl)phenyl)benzamide;
(S)-3,4-Dimethyl-N-(4-(piperidin-3-yl)phenyl)benzamide;
(R)-4-Chloro-2-fluoro-N-(4-(morpholin-2-yl)phenyl)benzamide;
(S)-4-Chloro-2-fluoro-N-(4-(morpholin-2-yl)phenyl)benzamide;
(RS)—N-(4-(Morpholin-2-yl)phenyl)-2-phenylthiazole-5-carboxamide;
(RS)—N-(4-(Morpholin-2-yl)phenyl)-2-phenylthiazole-4-carboxamide;
(S)—N-(4-(Piperidin-3-yl)phenyl)-3-(trifluoromethyl)benzamide;
(S)-4-(Methylthio)-N-(4-(piperidin-3-yl)phenyl)benzamide;
(S)-4-(Ethylthio)-N-(4-(piperidin-3-yl)phenyl)benzamide;
5-Chloro-pyrazine-2-carboxylic acid ((S)-4-piperidin-3-yl-phenyl)-amide;
5-Chloro-pyrazine-2-carboxylic acid ((R)-4-piperidin-3-yl-phenyl)-amide;
(S)—N-(4-(Piperidin-3-yl)phenyl)-6-(trifluoromethyl)nicotinamide;
(S)-6-Methyl-N-(4-(piperidin-3-yl)phenyl)nicotinamide;
(S)-6-(Methylthio)-N-(4-(piperidin-3-yl)phenyl)nicotinamide;
(RS)-6-Ethoxy-N-(4-(morpholin-2-yl)phenyl)nicotinamide;
(RS)—N-(4-(Morpholin-2-yl)phenyl)-2-phenyloxazole-4-carboxamide;
(S)—N-(4-(Piperidin-3-yl)phenyl)-5-(2,2,2-trifluoroethoxy)pyrazine-2-carboxamide;
(S)-5-Bromo-N-(4-(piperidin-3-yl)phenyl)pyrazine-2-carboxamide 2,2,2-trifluoroacetate;
(S)-6-Bromo-N-(4-(piperidin-3-yl)phenyl)nicotinamide 2,2,2-trifluoroacetate;
(S)-3-Methyl-N-(4-(piperidin-3-yl)phenyl)benzamide;
(S)-5-(Methylthio)-N-(4-(piperidin-3-yl)phenyl)pyrazine-2-carboxamide;
(S)-3-(Methylthio)-N-(4-(piperidin-3-yl)phenyl)benzamide;
(R)-3,4-Dimethyl-N-(4-(piperidin-3-yl)phenyl)benzamide;
(R)—N-(4-(Piperidin-3-yl)phenyl)-3-(trifluoromethyl)benzamide;
(R)-3-Chloro-N-(4-(morpholin-2-yl)phenyl)benzamide;
(S)-3-Chloro-N-(4-(morpholin-2-yl)phenyl)benzamide;
(R)—N-(4-(Piperidin-3-yl)phenyl)-6-(trifluoromethyl)nicotinamide;
(R)-4-(Methylthio)-N-(4-(morpholin-2-yl)phenyl)benzamide;
(S)-4-(Methylthio)-N-(4-(morpholin-2-yl)phenyl)benzamide;
(R)—N-(4-(Morpholin-2-yl)phenyl)-6-(2,2,2-trifluoroethoxy)nicotinamide;
(S)—N-(4-(Morpholin-2-yl)phenyl)-6-(2,2,2-trifluoroethoxy)nicotinamide;
(R)-2,6-Dichloro-N-(4-(morpholin-2-yl)phenyl)isonicotinamide;
(S)-2,6-Dichloro-N-(4-(morpholin-2-yl)phenyl)isonicotinamide;
(RS)-2-Chloro-6-methyl-N-(4-(morpholin-2-yl)phenyl)isonicotinamide;
(R)-4-Ethoxy-N-(4-(morpholin-2-yl)phenyl)benzamide;
(S)-4-Ethoxy-N-(4-(morpholin-2-yl)phenyl)benzamide;
(RS)-3-Methyl-N-(4-(morpholin-2-yl)phenyl)benzamide;
(RS)—N-(4-(Morpholin-2-yl)phenyl)-6-(pyrrolidin-1-yl)nicotinamide;
(RS)—N-(4-(Morpholin-2-yl)phenyl)-2-(trifluoromethyl)isonicotinamide;
(S)-2,6-Dichloro-N-(4-(piperidin-3-yl)phenyl)isonicotinamide;
(S)-2-Chloro-6-methyl-N-(4-(piperidin-3-yl)phenyl)isonicotinamide;
(R)—N-(4-(Morpholin-2-yl)phenyl)-6-(trifluoromethyl)nicotinamide;
(S)—N-(4-(Morpholin-2-yl)phenyl)-6-(trifluoromethyl)nicotinamide;
(R)-2-Chloro-6-methyl-N-(4-(morpholin-2-yl)phenyl)isonicotinamide;
(S)-2-Chloro-6-methyl-N-(4-(morpholin-2-yl)phenyl)isonicotinamide;
(RS)—N-(4-(Morpholin-2-yl)phenyl)-2-(pyrazin-2-yl)thiazole-4-carboxamide;
(S)—N-(4-(Piperidin-3-yl)phenyl)-6-propylnicotinamide;
(S)-6-Ethyl-N-(4-(piperidin-3-yl)phenyl)nicotinamide;
(RS)—N-(4-(Morpholin-2-yl)phenyl)-1-phenyl-1H-pyrazole-3-carboxamide;
(RS)-2-Ethoxy-N-(4-(morpholin-2-yl)phenyl)isonicotinamide;
(S)-4-Chloro-2-iodo-N-(4-(morpholin-2-yl)phenyl)benzamide;
(S)—N-(4-(1,4-Oxazepan-2-yl)phenyl)-3-chlorobenzamide;
3-Chloro-N-(4-((2S,6S)-6-methylmorpholin-2-yl)phenyl)benzamide;
(R)-4-Chloro-N-(4-(morpholin-2-yl)benzyl)benzamide;
(R)-6-Chloro-N-(4-(morpholin-2-yl)benzyl)nicotinamide;
3-Chloro-N-[4-((2S,5 S)-5-methyl-morpholin-2-yl)-phenyl]-benzamide;
3-Chloro-N-[4-((2S,5R)-5-methyl-morpholin-2-yl)-phenyl]-benzamide;
(RS)-(4-Pyrrolidin-3-yl-phenyl)-carbamic acid phenyl ester;
(RS)-(4-Pyrrolidin-3-yl-phenyl)-carbamic acid 4-fluoro-phenyl ester;
(RS)-(4-Pyrrolidin-3-yl-phenyl)-carbamic acid 4-chloro-phenyl ester;
(RS)-(4-Pyrrolidin-3-yl-phenyl)-carbamic acid 2-(4-fluoro-phenyl)-ethyl ester;
(RS)-(4-Pyrrolidin-3-yl-phenyl)-carbamic acid 4-fluoro-benzyl ester;
(RS)-(4-Pyrrolidin-3-yl-phenyl)-carbamic acid 2-(4-chloro-phenyl)-ethyl ester;
(RS)-(4-Pyrrolidin-3-yl-phenyl)-carbamic acid 2-(3-chloro-phenyl)-ethyl ester;
(RS)-(4-Pyrrolidin-3-yl-phenyl)-carbamic acid 2-(4-trifluoromethyl-phenyl)-ethyl ester;
(RS)-(4-Pyrrolidin-3-yl-phenyl)-carbamic acid 2-(3-trifluoromethyl-phenyl)-ethyl ester;
(RS)-(4-Pyrrolidin-3-yl-phenyl)-carbamic acid 2-(2,5-difluoro-phenyl)-ethyl ester;
(RS)-(4-Pyrrolidin-3-yl-phenyl)-carbamic acid 2-(4-trifluoromethoxy-phenyl)-ethyl ester;
(RS)-(4-Pyrrolidin-3-yl-phenyl)-carbamic acid 2-(3,4-dichloro-phenyl)-ethyl ester;
(RS)-(4-Pyrrolidin-3-yl-phenyl)-carbamic acid (RS)-1-(4-chloro-phenyl)-ethyl ester;
(RS)-(4-Pyrrolidin-3-yl-phenyl)-carbamic acid 3-(4-chloro-phenyl)-propyl ester;
(RS)-(4-Pyrrolidin-3-yl-phenyl)-carbamic acid indan-2-yl ester;
(RS)-(4-Pyrrolidin-3-yl-phenyl)-carbamic acid (RS)-1-(4-chloro-phenyl)-2,2,2-trifluoro-ethyl ester;
(S)-2,3-Dihydro-1H-inden-2-yl-4-(piperidin-3-yl)phenylcarbamate 2,2,2-trifluoroacetate;
(S)-2-(4-Chlorophenoxy)-N-(4-(piperidin-3-yl)phenyl)acetamide;
(S)-4-chlorobenzyl 4-(piperidin-3-yl)phenylcarbamate;
(RS)-2-(4-Chloro-phenyl)-N-(4-pyrrolidin-3-yl-phenyl)-acetamide;
(RS)—N—((RS)-4-Pyrrolidin-3-yl-phenyl)-2-(3-trifluoromethyl-phenyl)-propionamide;
(RS)—N-(4-Pyrrolidin-3-yl-phenyl)-2-(3-trifluoromethoxy-phenyl)-propionamide;
(RS)-3-(2-Chloro-phenyl)-N-(4-pyrrolidin-3-yl-phenyl)-propionamide;
   and
(RS)-3-(4-Chloro-phenyl)-N-(4-piperidin-3-yl-phenyl)-propionamide;
   or a pharmaceutically acceptable salt, a racemic mixture, an enantiomer and/or optical isomer (stereoisomer), a tautomer, a prodrug, or a solvate thereof.

In another aspect, useful compounds in the method of treating or preventing pain in a subject include:

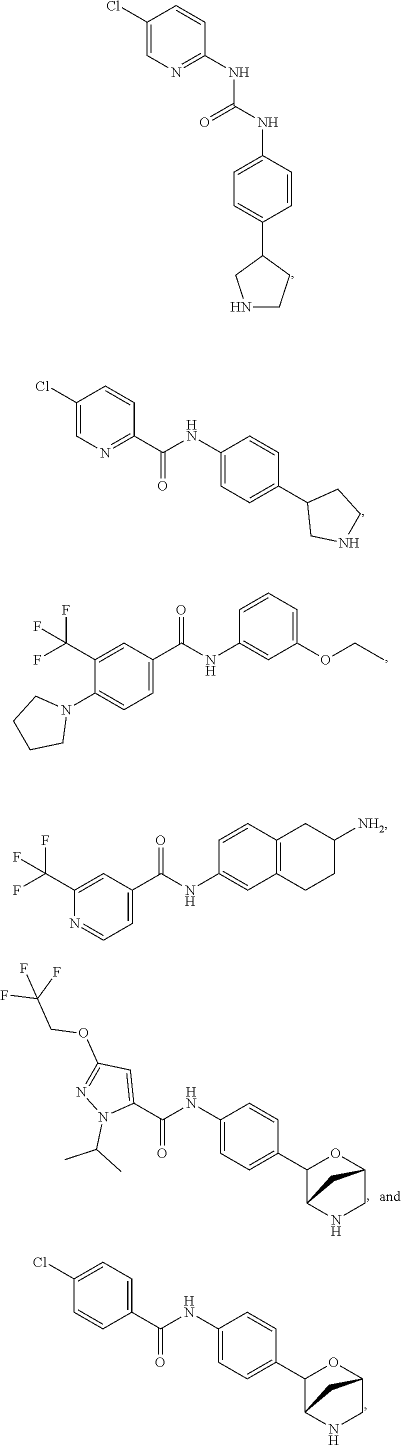 [SMILES: O=C(Nc1ccc(C2CCNC2)cc1)Nc1ccc(Cl)cn1 ; O=C(Nc1ccc(C2CCNC2)cc1)c1ccc(Cl)cn1 ; CCOc1cccc(NC(=O)c2ccc(N3CCCC3)c(C(F)(F)F)c2)c1 ; NC1CCc2cc(NC(=O)c3ccnc(C(F)(F)F)c3)ccc2C1 ; CC(C)n1nc(OCC(F)(F)F)cc1C(=O)Nc1ccc(C2O[C@@H]3CN[C@H]2C3)cc1 ; O=C(Nc1ccc(C2O[C@@H]3CN[C@H]2C3)cc1)c1ccc(Cl)cc1]

or a pharmaceutically acceptable salt, a racemic mixture, an enantiomer and/or optical isomer (stereoisomer), a tautomer, a prodrug, or a solvate thereof.

The compounds described herein can be prepared according to methods known in the art, for example, the methods as described in U.S. Patent Application Publication No. 2011/0152245 A1 and International Patent Application Publication No. 2012/016879 A1. These patent documents are incorporated herein in their entireties.

In another aspect, the present disclosure provides a method of treating pain in a subject, comprising administering to a subject in need of such treatment a therapeutically effective amount of a compound of Formula (IV):

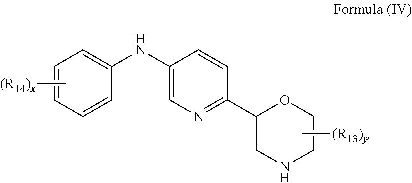

or a pharmaceutically acceptable salt, a racemic mixture, an enantiomer and/or optical isomer (stereoisomer), a tautomer, a prodrug, or a solvate thereof,

wherein:

R₁₃ is independently hydrogen, halogen, alkyl, alkoxy, haloalkyl, haloalkoxy, hydroxyl, cyano, amino, cycloalkyl, heterocycloalkyl, aryl, or heteroaryl;

R₁₄ is independently hydrogen, halogen, alkyl, alkoxy, haloalkyl, haloalkoxy, hydroxyl, cyano, amino, cycloalkyl, heterocycloalkyl, aryl, or heteroaryl;

x is 0, 1, or 2; and

y is 0, 1, or 2.

In one embodiment in the compound of Formula (IV), R₁₄ is hydrogen, halogen, alkyl, or haloalkyl.

In one embodiment in the compound of Formula (IV), R₁₃ is hydrogen, halogen, alkyl, or haloalkyl.

In one embodiment in the compound of Formula (IV), R₁₄ is halogen.

In one embodiment in the compound of Formula (IV), R₁₄ is chlorine.

In one embodiment, the compound of Formula (IV) is of the structure:

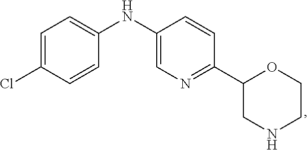 [SMILES: Clc1ccc(Nc2ccc(C3CNCCO3)nc2)cc1]

or a pharmaceutically acceptable salt, a racemic mixture, an enantiomer and/or optical isomer (stereoisomer), a tautomer, a prodrug, or a solvate thereof.

### VI. Testing TAAR1 Agonists or Partial Agonists for their Therapeutic Effects

TAAR1 agonists or partial agonists of the present disclosure can be tested for their therapeutic effect of treatment, amelioration or prevention of pain in animal models.

Test Animals:

Each experiment uses rats weighing between 200-410 g at the start of the experiment. The rats are group-housed and have free access to food and water at all times, except prior to oral administration of a compound of the present disclosure when food is removed for about 16 hours before dosing. A control group acts as a comparison to rats treated with a compound of the present disclosure. The control group is administered the vehicle for the compound of the present disclosure. The volume of vehicle administered to the control group is the same as the volume of carrier and compound of the present disclosure administered to the test group.

Acute Pain Tests

Tail Flick Tests:

To assess the actions of a compound of the present disclosure for the treatment or prevention of acute pain, the rat tail flick can be used. Rats are gently restrained by hand and the tail exposed to a focused beam of radiant heat at a point 5 cm from the tip using a tail flick unit (Model 7360, commercially available from Ugo Basile of Italy). Tail flick latencies are defined as the interval between the onset of the thermal stimulus and the flick of the tail. Animals not responding within 20 seconds are removed from the tail flick unit and assigned a withdrawal latency of 20 seconds. Tail flick latencies are measured immediately before (pre-treatment) and 1, 3, and 5 hours following administration of a compound of the present disclosure. Data are expressed as tail flick latency(s) and the percentage of the maximal possible effect (% MPE), i.e., 20 seconds, is calculated as follows:

$$\% MPE = \frac{\begin{matrix}[ ( post administration latency ) - \\ ( pre \text{-} administration latency ) ]\end{matrix}}{( 20 s - pre \text{-} administration latency )} \times 100 \qquad$$

The rat tail flick test is described in F. E. D'Amour et al., “A Method for Determining Loss of Pain Sensation,”. Pharmacol. Exp. Ther. 72:74-79 (1941).

Hot Plate Tests:

To assess the actions of a compound of the present disclosure for the treatment or prevention of acute pain, the rat hot plate test can also be used. Rats are tested using a hot plate apparatus consisting of a clear plexiglass cylinder with a heated metal floor maintained at a temperature of 48-52° C. (Model 7280, commercially available from Ugo Basile of Italy). A rat is placed into the cylinder on the hot plate apparatus for a maximum duration of 30 seconds, or until it exhibits a nocifensive behavior (behavioral endpoint), at which time it is removed from the hot plate, and the response latency recorded. Hot plate latencies are measured immediately before (pre-treatment) and 1, 3, and 5 hours following administration of a compound of the present disclosure. The nocifensive behavioral endpoint is defined as any of the following: 1) paw withdrawal, either as a sustained lift or with shaking or licking; 2) alternating foot lifting; 3) escape or attempted escape from the testing device; or 4) vocalization. Data are expressed as response latency(s) and the percentage of the maximal possible effect is calculated as described above for the tail flick test with the exception of 30 seconds being used for the cut-off value in the denominator. The hot plate test is described in G. Woolfe and A. D. MacDonald, J. Pharmacol. Exp. Ther. 80:300-307 (1944).

Inflammatory Pain:

To assess the actions of a compound of the present disclosure for the treatment or prevention of inflammatory pain, the Freund's complete adjuvant (“FCA”) model of inflammatory pain can be used. FCA-induced inflammation of the rat hind paw is associated with the development of persistent inflammatory mechanical hyperalgesia and provides reliable prediction of the anti-hyperalgesic action of clinically useful analgesic drugs (L. Bartho et al., “Involvement of Capsaicin-sensitive Neurones in Hyperalgesia and Enhanced Opioid Antinociception in Inflammation,” Naunyn-Schmiedeberg's Archives of Pharmacol. 342:666-670 (1990)). The left hind paw of each animal is administered a 50 μL intraplantar injection of 50% FCA. Prior to injection of FCA (baseline) and 24 hour post injection, the animal is assessed for response to noxious mechanical stimuli by determining the PWT, as described below. Rats are then administered compounds orally or intraperitoneally an amount between 3-30 mg/kg in the present disclosure, 30 mg/kg of Celebrex, indomethacin or naproxen (positive control), or vehicle. Responses to noxious mechanical stimuli are determined 1, 3, 5 and 24 hours post administration. Percentage reversal of hyperalgesia for each animal is defined as:

$$\% Reversal = \frac{\begin{matrix}[ ( post administration PWT ) - \\ ( pre \text{-} administration PWT ) ]\end{matrix}}{[ ( baseline PWT - pre \text{-} administration PWT ) ]} \times 100$$

Neuropathic Pain:

To assess the actions of a compound of the present disclosure for the treatment or prevention of neuropathic pain, the Chung or Spinal Nerve Ligation (SNL) model will be used.

In the spinal nerve ligation (“SNL”) model, neuropathic pain is demonstrated by producing mechanical hyperalgesia, thermal hyperalgesia and tactile allodynia in rats. Surgery is performed under isoflurane/O₂ inhalation anesthesia. Following induction of anesthesia, a 3 centimeter incision is made and the left paraspinal muscles are separated from the spinous process at the L₄-S₂ levels. The L₆ transverse process is carefully removed with a pair of small rongeurs to identify visually the L₄-L₆ spinal nerves. The left L₅ (or L₅ and L₆) spinal nerve(s) is isolated and tightly ligated with silk thread. A complete hemostasis is confirmed and the wound is sutured using non-absorbable sutures, such as nylon sutures or stainless steel staples. Sham-rats undergo an identical surgical procedure except that the spinal nerve(s) is not manipulated. Following surgery animals are weighed, administered a subcutaneous (SC) injection of saline or ringers lactate, the wound area is dusted with antibiotic powder and they are kept on a warm pad until they recover from the anesthesia. Animals are then returned to their home cages until behavioral testing begins (i.e., 3-4 weeks post-surgery). The animals are assessed for their response to either a noxious or non-noxious stimuli, described below, prior to surgery (baseline), then 1, 3, and 5 hours after being administered a compound of the present disclosure 3 or 4 weeks post-surgery.

Nociceptive Pain:

To assess the actions of compounds of the present disclosure for the treatment or prevention of nociceptive pain, the hot plate and tail flick assays will be used as described previously. Nociceptive pain assessment can be used to distinguish compounds of the present disclosure as being analgesics or neuropathics.

Response to Mechanical Stimuli as an Assessment of Mechanical Hyperalgesia:

The paw pressure assay can be used to assess mechanical hyperalgesia. Sensitivity to noxious mechanical stimuli is measured in animals using the paw pressure test to assess mechanical hyperalgesia. In rats, hind paw withdrawal thresholds (“PWT”), measured in grams, in response to a noxious mechanical stimulus are determined using an analgesymeter (Model 7200, commercially available from Ugo Basile of Italy), as described in C. Stein, “Unilateral Inflammation of the Hindpaw in Rats as a Model of Prolonged Noxious Stimulation: Alterations in Behavior and Nociceptive Thresholds,” Pharmacol. Biochem. and Behavior 31:451-455 (1988). The rat is gently restrained, its hindpaw is placed on a small round platform, and punctate pressure is applied to the dorsal surface of the hindpaw in a graded manner. The maximum weight that is applied to the hind paw is set at 250 grams and the end point is taken as complete withdrawal of the paw. PWT is determined once for each rat at each time point and either only the affected (ipsilateral; same side as the injury) rear paw is tested, or both the ipsilateral and contralateral (non-injured; opposite to the injury) rear paw are tested. Rats are tested prior to surgery or FCA administration to determine a baseline, or normal, PWT. Rats are tested again 3-4 weeks post-surgery or 24 hours post-FCA injection and at different times after (e.g., 1, 3, and 5 hours) compound or vehicle administration. A sham control is also implemented to determine the PWT of a rat having neither surgery nor compound administration. An increase in PWT following drug administration indicates that the test compound reduces mechanical hyperalgesia.

Response to Thermal Stimuli as an Assessment of Thermal Hyperalgesia:

The plantar test can be used to assess thermal hyperalgesia of an injured (i.e., SNL) or injected paw (i.e., FCA). For this test, hind paw withdrawal latencies to a noxious thermal stimulus applied to the plantar surface of the hindpaw are determined using a plantar test apparatus (commercially available from Ugo Basile of Italy) following the technique described by K. Hargreaves et al., “A New and Sensitive Method for Measuring Thermal Nociception in Cutaneous Hyperalgesia,” Pain 32(1):77-88 (1988). The maximum exposure time is set at 32 seconds to avoid tissue damage and any directed paw withdrawal from the heat source is taken as the end point. Three latencies are determined at each time point and averaged. Only the affected (ipsilateral) paw is tested, or both the ipsilateral and contralateral (non-injured) paw are tested.

Assessment of Tactile Allodynia:

To assess tactile allodynia, rats are placed in clear, plexiglass compartments with a wire mesh floor and allowed to habituate for a period of at least 15 minutes. After habituation, a series of von Frey monofilaments are presented to the plantar surface of the affected (ipsilateral) foot of each rat. Briefly, calibrated filaments will be applied to the plantar surface of the left hind paw through wire screen mesh flooring just sufficient enough to bend the microfilament. Testing will begin with a 2.0 g (number 3.14) von Frey monofilament. If a positive withdrawal response (licking/grooming/flinching) is noted, the next finer microfilament will be tested. Conversely, if a response is not generated, then the next thicker monofilament will be used. Paw withdrawal thresholds will be calculated according to the methods of Dixon (1980) and Chaplan et al. (1994) and will be determined for the injured paw only to avoid the confound of the development of hypersensitivity in the contralateral limb as this has been described by a number of groups (Hatashita et al., 2008; von Banchet et al., 2009). Those animals exhibiting thresholds <10 g of force will be excluded from surgery. For those animals undergoing surgery, tactile thresholds will be re-assessed. Only those Sham animals displaying a threshold >10 g and SNL animals displaying sensitivities <5 g will be used. Flinching, paw withdrawal or licking of the paw are considered nociceptive behavioral responses. (See e.g., Chaplan S R, Bach F W, Pogrel J W, Chung J M, Yaksh T L. Quantitative assessment of tactile allodynia in the rat paw. J Neurosci Methods 53:55-63, 1994; Dixon W J. Efficient analysis of experimental observations. Annu Rev Pharmacol Toxicol 20: 441-462,1980; Hatashita S, Sekiguchi M, Kobayashi H, Konno S, Kikuchi S. Contralateral neuropathic pain and neuropathology in dorsal root ganaglion and spinal cord following hemilateral nerve injury in rats. Spine 33(12):1344-1351, 2008; Von Banchet G, Boettger M K, Fisher N, Gajda M, Brauer R, Schaible H G. Experimental arthritis causes tumor necrosis factor-alpha-dependent infiltration of macrophages into rat dorsal root ganglia which correlates with pain-related behavior. Pain 145:151-159, 2009.)

### VII. Methods of Treating or Preventing Acute-Chronic Neuropathic Pain

The TAAR1 agonists or partial agonists of the present disclosure can be used for treating acute, chronic, or neuropathic pain.

Acute pain includes, but is not limited to, perioperative pain, postoperative pain, post-traumatic pain, acute disease related pain, and pain related to diagnostic procedures, orthopedic manipulations, and myocardial infarction. Acute pain in the perioperative setting includes pain because of pre-existing disease, the surgical procedure, e.g., associated drains, chest or nasogastric tubes, or complications, or a combination of disease-related and procedure-related sources.

Neuropathic pain is a heterogeneous disease state with an unclear etiology. Neuropathic pain can be mediated by multiple mechanisms. This type of pain generally arises from injury to the peripheral or central nervous tissue. The syndromes include pain associated with spinal cord injury, multiple sclerosis, post-herpetic neuralgia, trigeminal neuralgia, phantom pain, causalgia, and reflex sympathetic dystrophy and lower back pain. The chronic neuropathic pain patients suffer the abnormal pain sensations that can be described as spontaneous pain, continuous superficial burning and/or deep aching pain. The pain can be evoked by heat-, cold-, and mechano-hyperalgesia or by heat-, cold-, or non-noxious stimuliallodynia (i.e., touch, clothing).

Neuropathic pain can be caused by injury or infection of peripheral sensory nerves. It includes, but is not limited to pain from peripheral nerve trauma, herpes virus infection, diabetes mellitus, causalgia, plexus avulsion, neuroma, limb amputation, and vasculitis. Neuropathic pain can also be caused by nerve damage from chronic alcoholism, human immunodeficiency virus infection, hypothyroidism, uremia, or vitamin deficiencies. Stroke (spinal or brain) and spinal cord injury can also induce neuropathic pain. Cancer-related neuropathic pain results from tumor growth compression of adjacent nerves, brain, or spinal cord. In addition, cancer treatments, including chemotherapy and radiation therapy, can cause nerve injury. Neuropathic pain includes but is not limited to pain caused by nerve injury such as, for example, the pain from which diabetics suffer.

The dosage amount of the TAAR1 agonists or partial agonists of the present disclosure that is effective for the treatment or prevention of pain can be determined by standard clinical techniques. In addition, in vitro and/or in vivo assays can optionally be employed to help identify optimal dosage ranges. The precise dose to be employed will also depend on, e.g., the route of administration and the extent of the condition to be treated, and can be decided according to the judgment of a practitioner and/or each subject's circumstances. Variations in dosing may occur depending upon typical factors such as the weight, age, gender and physical condition (e.g., hepatic and renal function) of the subject being treated, the affliction to be treated, the severity of the symptoms, the frequency of the dosage interval, and the presence of any deleterious side-effects, among other things.

In one embodiment, a TAAR1 agonist or partial agonist of the present disclosure is administered to a subject by any appropriate route, such as intradermal, intramuscular, intraperitoneal, parenteral, intravenous, subcutaneous, intranasal, epidural, oral, sublingual, buccal, intracerebral, intravaginal, transdermal, transmucosal, rectal, by inhalation, or topical (particularly to the ears, nose, eyes, or skin), at a dosage amount ranging from about 0.0025 to about 1500 mg/per kg body weight/day, from about 0.0025 to about 1000 mg/per kg body weight/day, from about 0.0025 to about 500 mg/per kg body weight/day, from about 0.0025 to about 100 mg/per kg body weight/day, from about 0.0025 to about 50 mg/per kg body weight/day, from about 0.01 to about 100 mg/per kg body weight/day, from about 0.01 to about 50 mg/per kg body weight/day, or from about 0.01 to about 10 mg/per kg body weight/day.

In one embodiment, a TAAR1 agonist or partial agonist of the present disclosure is administered to a subject by any appropriate route, such as intradermal, intramuscular, intraperitoneal, parenteral, intravenous, subcutaneous, intranasal, epidural, oral, sublingual, buccal, intracerebral, intravaginal, transdermal, transmucosal, rectal, by inhalation, or topical (particularly to the ears, nose, eyes, or skin), in a unit dose, containing from about 0.01 to about 1500 mg, from about 0.01 to about 1000 mg, from about 0.1 to about 500 mg, or from about 10 to about 500 mg of the TAAR1 agonist or partial agonist. The unit dose can be administered one or more times a day. In one embodiment, a TAAR1 agonist or partial agonist of the present disclosure is administered to a subject in an unit oral dose containing from about 0.01 to about 1500 mg of the TAAR1 agonist or antagonist.

### VIII. Pharmaceutical Compositions

TAAR1 agonists or partial agonists of the present disclosure are useful for treating or preventing acute-chronic neuropathic pain in a subject in need thereof. The subjects include humans or animals, although the invention is not intended to be so limited.

When administered to a human subject, the TAAR1 agonists or partial agonists of the present disclosure can be administered as a component of a composition that comprises a pharmaceutically acceptable carrier or excipient, by any appropriate route, such as intradermal, intramuscular, intraperitoneal, parenteral, intravenous, subcutaneous, intranasal, epidural, oral, sublingual, buccal, intracerebral, intravaginal, transdermal, transmucosal, rectal, by inhalation, or topical (particularly to the ears, nose, eyes, or skin). Delivery can be either local or systemic. In certain embodiments, administration will result in the release of the TAAR1 agonists or partial agonists of the present disclosure into the bloodstream.

Pharmaceutical compositions can take the form of solutions, suspensions, emulsions, tablets, pills, pellets, powders, multi-particulates, capsules, capsules containing liquids, capsules containing powders, capsules containing multi-particulates, lozenges, sustained-release formulations, suppositories, transdermal patches, transmucosal films, sub-lingual tablets or tabs, aerosols, sprays, or any other form suitable for use. In one embodiment, the composition is in the form of a tablet. In another embodiment, the composition is in the form of a capsule. Other examples of suitable pharmaceutical excipients are described in Remington's Pharmaceutical Sciences 1447-1676 (Alfonso R. Gennaro ed., 19th ed. 1995), incorporated herein by reference in its entirety.

Pharmaceutical compositions comprise a suitable amount of a pharmaceutically acceptable excipient so as to provide the form for proper administration to a subject. Such a pharmaceutical excipient can be a diluent, suspending agent, solubilizer, binder, disintegrant, preservative, coloring agent, lubricant, and the like. The pharmaceutical excipient can be a liquid, such as water or an oil, including those of petroleum, animal, vegetable, or synthetic origin, such as peanut oil, soybean oil, mineral oil, sesame oil, and the like. The pharmaceutical excipient can be saline, gum acacia, gelatin, starch paste, talc, keratin, colloidal silica, urea, and the like. In addition, auxiliary, stabilizing, thickening, lubricating, and coloring agents can be used. In one embodiment, the pharmaceutically acceptable excipient is sterile when administered to a human subject. In another embodiment, water, saline, and dextrose are excipients when TAAR1 agonists or partial agonists of the present disclosure are administered intravenously. Aqueous dextrose and glycerol solutions can also be employed as liquid excipients, particularly for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol, and the like. The pharmaceutical compositions, if desired, can also contain minor amounts of wetting or emulsifying agents, or pH buffering agents. Specific examples of pharmaceutically acceptable carriers and excipients that can be used to formulate oral dosage forms are described in the Handbook of Pharmaceutical Excipients, American Pharmaceutical Association (1986).

In certain embodiments, the TAAR1 agonists or partial agonists of the present disclosure are formulated for oral administration in the form of tablets, capsules, gelcaps, caplets, lozenges, aqueous or oily solutions, suspensions, granules, powders, emulsions, syrups, or elixirs, for example. The tablets can be compressed, enteric-coated, sugar-coated, film-coated, multiply compressed or multiply layered.

An orally administered TAAR1 agonist or partial agonist of present disclosure can contain one or more additional agents such as, for example, sweetening agents such as fructose, aspartame or saccharin; flavoring agents such as peppermint, oil of wintergreen, or cherry; coloring agents; and preserving agents, and stabilizers, to provide stable, pharmaceutically palatable dosage forms. Techniques and compositions for making solid oral dosage forms are described in Pharmaceutical Dosage Forms: Tablets (Lieberman, Lachman and Schwartz, eds., 2nd ed.) published by Marcel Dekker, Inc. Techniques and compositions for making tablets (compressed and molded), capsules (hard and soft gelatin) and pills are also described in Remington's Pharmaceutical Sciences 1553-1593 (Arthur Osol, ed., 16^{th} ed., Mack Publishing, Easton, Pa. 1980). Liquid oral dosage forms include aqueous and nonaqueous solutions, emulsions, suspensions, and solutions and/or suspensions reconstituted from non-effervescent granules, optionally containing one or more suitable solvents, preservatives, emulsifying agents, suspending agents, diluents, sweeteners, coloring agents, flavoring agents, and the like. Techniques and compositions for making liquid oral dosage forms are described in Pharmaceutical Dosage Forms: Disperse Systems, (Lieberman, Rieger and Banker, eds.) published by Marcel Dekker, Inc.

When the TAAR1 agonists or partial agonists of the present disclosure are formulated for parenteral administration by injection (e.g., continuous infusion or bolus injection), the formulation can be in the form of a suspension, solution, or emulsion in an oily or aqueous vehicle, and such formulations can further comprise pharmaceutically necessary additives such as one or more stabilizing agents, suspending agents, dispersing agents, and the like. When a TAAR1 agonist or partial agonist of the present disclosure is to be injected parenterally, it can be, e.g., in the form of an isotonic sterile solution. The TAAR1 agonists or partial agonists of the present disclosure can also be in the form of a powder for reconstitution as an injectable formulation.

In certain embodiments, the TAAR1 agonists or partial agonists of the present disclosure are formulated into a pharmaceutical composition for intravenous administration. Typically, such compositions comprise sterile isotonic aqueous buffer. Where necessary, the compositions can also include a solubilizing agent. Generally, the ingredients are supplied either separately or mixed together in unit dosage form, for example, as a dry lyophilized powder or water free concentrate in a hermetically sealed container such as an ampule or sachette indicating the quantity of active agent. Where administered by infusion, the compositions can be dispensed, for example, with an infusion bottle containing sterile pharmaceutical grade water or saline. Where administered by injection, an ampule of sterile water for injection or saline can be provided so that the ingredients can be mixed prior to administration.

When the TAAR1 agonists or partial agonists of the present disclosure are administered by inhalation, they can be formulated into a dry aerosol, or an aqueous or partially aqueous solution.

In one embodiment, the TAAR1 agonists or partial agonists of the present disclosure can be delivered in a vesicle, in particular a liposome (see Langer, Science 249:1527-1533 (1990); and Treat et al., Liposomes in the Therapy of Infectious Disease and Cancer 317-327 and 353-365 (1989)).

In certain embodiments, the TAAR1 agonists or partial agonists of the present disclosure are administered locally. This can be achieved, for example, by local infusion during surgery, topical application, e.g., in conjunction with a wound dressing after surgery, by injection, by means of a catheter, by means of a suppository or enema, or by means of an implant, said implant being of a porous, non-porous, or gelatinous material, including membranes, such as sialastic membranes, or fibers.

In certain embodiments, the TAAR1 agonists or partial agonists of the present disclosure are delivered in an immediate release form. In other embodiments, the TAAR1 agonists or partial agonists of the present disclosure are delivered in a controlled-release system or sustained-release system. Controlled- or sustained-release pharmaceutical compositions can have a common goal of improving drug therapy over the results achieved by their non-controlled or non-sustained-release counterparts. Advantages of controlled- or sustained-release compositions include extended activity of the drug, reduced dosage frequency, and increased compliance. In addition, controlled- or sustained-release compositions can favorably affect the time of onset of action or other characteristics, such as blood levels of the drug compound, and can thus reduce the occurrence of adverse side effects.

Controlled- or sustained-release compositions can initially immediately release an amount of a TAAR1 agonist or partial agonist of the present disclosure that promptly produces the desired therapeutic or prophylactic effect, and gradually and continually release the remaining amounts to maintain a level of therapeutic or prophylactic effect over an extended period of time. Controlled- or sustained-release of an active ingredient can be stimulated by various conditions, including but not limited to, changes in pH, changes in temperature, concentration or availability of enzymes, concentration or availability of water, or other physiological conditions or compounds.

Controlled-release and sustained-release means for use according to the present disclosure may be selected from those known in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,566, each of which is incorporated herein by reference. Such dosage forms can be used to provide controlled- or sustained-release of one or more active ingredients using, for example, hydroxypropylmethyl cellulose, other polymer matrices, gels, permeable membranes, osmotic systems, multilayer coatings, microparticles, multiparticulates, liposomes, microspheres, or a combination thereof to provide the desired release profile in varying proportions. Suitable controlled- or sustained-release formulations known in the art, including those described herein, can be readily selected for use with the active ingredients of the invention in view of this disclosure. See also Goodson, “Dental Applications” (pp. 115-138) in Medical Applications of Controlled Release, Vol. 2, Applications and Evaluation, R. S. Langer and D. L. Wise eds., CRC Press (1984). Other controlled- or sustained-release systems that are discussed in the review by Langer, Science 249:1527-1533 (1990) can be selected for use according to the present disclosure. In one embodiment, a pump can be used (Langer, Science 249:1527-1533 (1990); Sefton, CRC Crit. Ref Biomed. Eng. 14:201 (1987); Buchwald et al., Surgery 88:507 (1980); and Saudek et al., N. Engl. J. Med. 321:574 (1989)). In another embodiment, polymeric materials can be used (see Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 23:61 (1983); Levy et al., Science 228:190 (1985); During et al., Ann. Neurol. 25:351 (1989); and Howard et al., J. Neurosurg. 71:105 (1989)). In yet another embodiment, a controlled- or sustained-release system can be placed in proximity of a treatment target, e.g., the spinal column, or brain, and thus requiring only a fraction of the systemic dose.

When the TAAR1 agonists or partial agonists of the present disclosure are in a tablet or a pill form, the tablet or pill can be coated to delay disintegration and absorption in the gastrointestinal tract, thereby providing a sustained action over an extended period of time. Selectively permeable membranes surrounding an osmotically active driving compound are also suitable for orally administered compositions. In these latter platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be used. Oral compositions can include standard excipients such as mannitol, lactose, starch, magnesium stearate, sodium saccharin, cellulose, and magnesium carbonate. In one embodiment, the excipients are of pharmaceutical grade.

Pharmaceutical compositions of the present disclosure include single unit dosage forms suitable for oral administration such as, but not limited to, tablets, capsules, gelcaps, and caplets that are adapted for controlled- or sustained-release.

The following examples are illustrative, but not limiting, of the methods of the present disclosure. Suitable modifications and adaptations of the variety of conditions and parameters normally encountered in clinical therapy and which are obvious to those skilled in the art in view of this disclosure are within the spirit and scope of the disclosure.

### EXAMPLES

### Example 1. SNL-Induced Mechanical Hyperalgesia

Representative agonists and partial agonists of the present disclosure are tested in the spinal nerve ligation (“SNL”)-induced mechanical hyperalgesia model in male, Sprague-Dawley rats. Sensitivity to noxious mechanical stimuli is measured in animals using the paw pressure test to assess mechanical hyperalgesia. In rats, hind paw withdrawal thresholds (“PWT”), measured in grams, in response to a noxious mechanical stimulus are determined using an analgesymeter (Model 7200, commercially available from Ugo Basile of Italy), as described in C. Stein, “Unilateral Inflammation of the Hindpaw in Rats as a Model of Prolonged Noxious Stimulation: Alterations in Behavior and Nociceptive Thresholds,” Pharmacol. Biochem. & Behavior 31:451-455 (1988). Briefly, the rat's paw is placed on a small platform, and weight is applied in a graded manner up to a maximum of 250 grams. The endpoint is taken as the weight at which the paw is completely withdrawn. PWT is determined once for each rat at each time point. PWT is measured only in the injured paw. Rats are tested prior to surgery to determine a baseline, or normal, PWT. Rats are tested again 3-4 weeks post-surgery and at different times after (e.g., 1, 3, and 5 hours) compound administration. Gabapentin is used as the positive control. A SNL control is implemented to determine the PWT of a rat having no drug administration. A sham control is implemented to determine the PWT of a rat having neither surgery nor drug administration (i.e., vehicle administration). An increase in PWT following drug administration indicates that the test compound reduces mechanical hyperalgesia. Data are analyzed by a two-way analysis of variance (“ANOVA”) followed by a Bonferroni multiples comparisons test.

### Example 2. The Effect of TAAR1 Partial Agonist Compound 1 on SNL-Induced Mechanical Hyperalgesia in Rats Following Oral (“PO”) Dosing

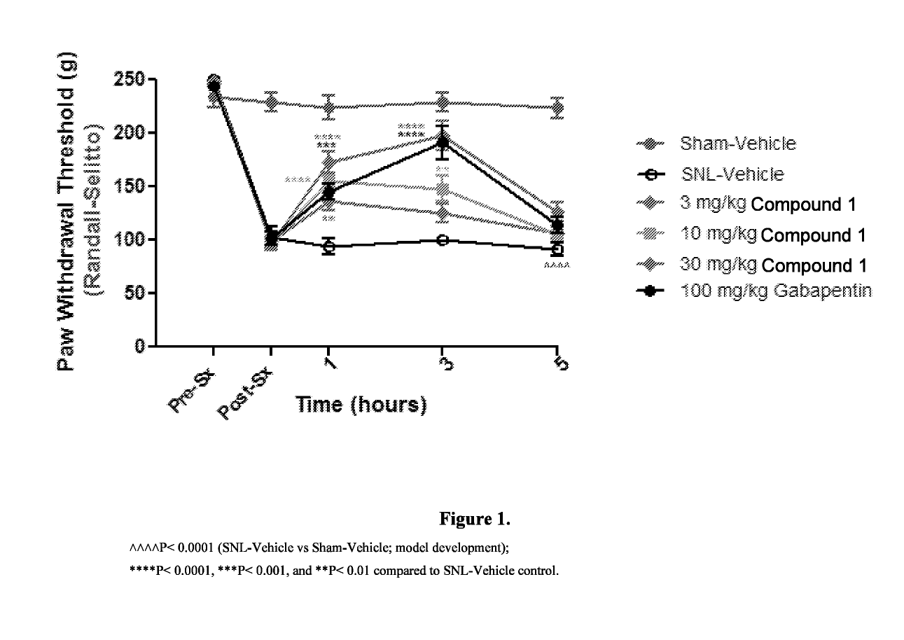

Compound 1 was tested using the procedures described in Example 1. Compound 1 reduced SNL-induced mechanical hyperalgesia in rats when dosed orally at ≤3 mg/kg one hour before testing. Compound 1 showed robust efficacy in the SNL-induced mechanical hyperalgesia model of neuropathic pain in rats, with 69% maximum reversal achieved at 3 hours following PO dosing (see FIG. 1). Efficacy was most notable at a dosing of 30 mg/kg; the minimum effective dose (“MED”) was ≤3 mg/kg. In the experiment, male, Sprague-Dawley rats, 207-331 g (n=8-10/group) were baselined for left hind paw threshold before (Pre-Sx) and then 3 weeks post-SNL surgery. Compound 1 (PO) and gabapentin (intraperitoneal), the positive control, were administered 1 hour prior to testing. Compound 1 was formulated in 0.5% methyl-cellulose while gabapentin was dissolved in 0.9% normal saline.

### Example 3. The Effect of TAAR1 Partial Agonist Compound 1 on SNL-Induced Mechanical Hyperalgesia in Rats Following Intraperitoneal (“IP”) Dosing

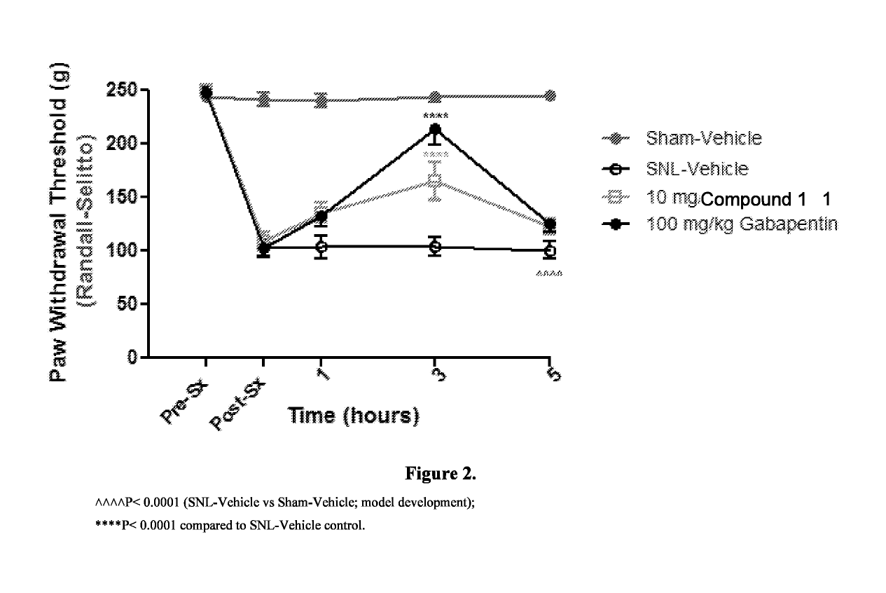

Compound 1 was tested using the procedures described in Example 1. Compound 1 reduced SNL-induced mechanical hyperalgesia in rats when dosed intraperitoneally at 10 mg/kg one hour before testing. Compound 1 showed robust efficacy in the SNL-induced mechanical hyperalgesia model of neuropathic pain in rats, with 41% maximum reversal achieved at 3 hours following IP dosing (see FIG. 2). In the experiment, male, Sprague-Dawley rats, 270-360 g (n=8-9/group) were baselined for left hind paw threshold before (Pre-Sx) and then 4 weeks post-SNL surgery. Compound 1 (IP) and gabapentin (IP), the positive control, were administered 1 hour prior to testing. Compound 1 was formulated in 25% hydroxypropyl-β-cyclodextrin while gabapentin was dissolved in 0.9% normal saline.

### Example 4. The Effect of TAAR1 Agonist Compound 2 on SNL-Induced Mechanical Hyperalgesia in Rats Following IP Dosing

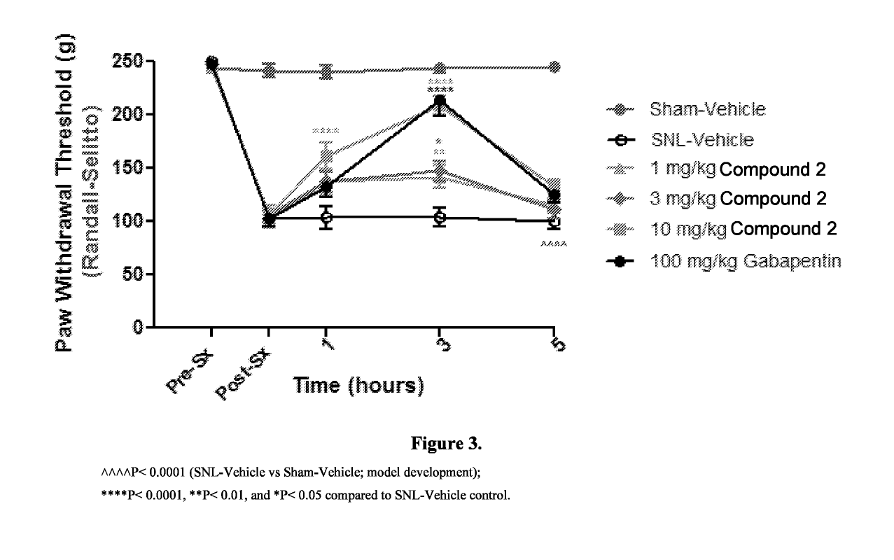

Compound 2 was tested using the procedures described in Example 1. Compound 2 reduced SNL-induced mechanical hyperalgesia in rats when dosed IP at ≤1 mg/kg one hour before testing. Compound 2 showed robust efficacy in the SNL-induced mechanical hyperalgesia model of neuropathic pain in rats, with 76% maximum reversal achieved at 3 hours following IP dosing (see FIG. 3). Efficacy was most notable at a dosing of 10 mg/kg; the MED was ≤1 mg/kg. In the experiment, male, Sprague-Dawley rats, 270-360 g (n=8-9/group) were baselined for left hind paw threshold before (Pre-Sx) and then 4 weeks post-SNL surgery. Compound 2 (IP) and gabapentin (IP), the positive control, were administered 1 hour prior to testing. Compound 2 was formulated in 25% hydroxypropyl-β-cyclodextrin while gabapentin was dissolved in 0.9% normal saline.

### Example 5. The Effect of TAAR1 Partial Agonist Compound 3 on SNL-Induced Mechanical Hyperalgesia in Rats Following PO Dosing

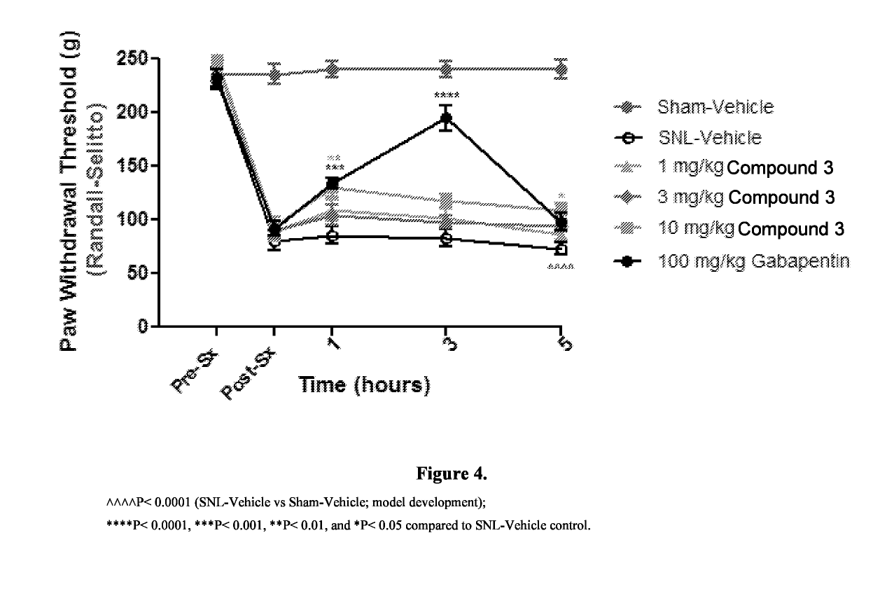

Compound 3 was tested using the procedures described in Example 1. Compound 3 reduced SNL-induced mechanical hyperalgesia in rats when dosed orally at 10 mg/kg one hour before testing. Compound 3 showed robust efficacy in the SNL-induced mechanical hyperalgesia model of neuropathic pain in rats, with 23% maximum reversal achieved at 1 hour following PO dosing (see FIG. 4). Efficacy was most notable at a dosing of 10 mg/kg; the MED was 10 mg/kg. In the experiment, male, Sprague-Dawley rats, 289-359 g (n=6-10/group) were baselined for left hind paw threshold before (Pre-Sx) and then 4 weeks post-SNL surgery. Compound 3 (PO) and gabapentin (IP), the positive control, were administered 1 hour prior to testing. Compound 3 was formulated in 0.5% methyl-cellulose while gabapentin was dissolved in 0.9% normal saline.

### Example 6. The Effect of TAAR1 Partial Agonist Compound 3 on SNL-Induced Mechanical Hyperalgesia in Rats Following IP Dosing

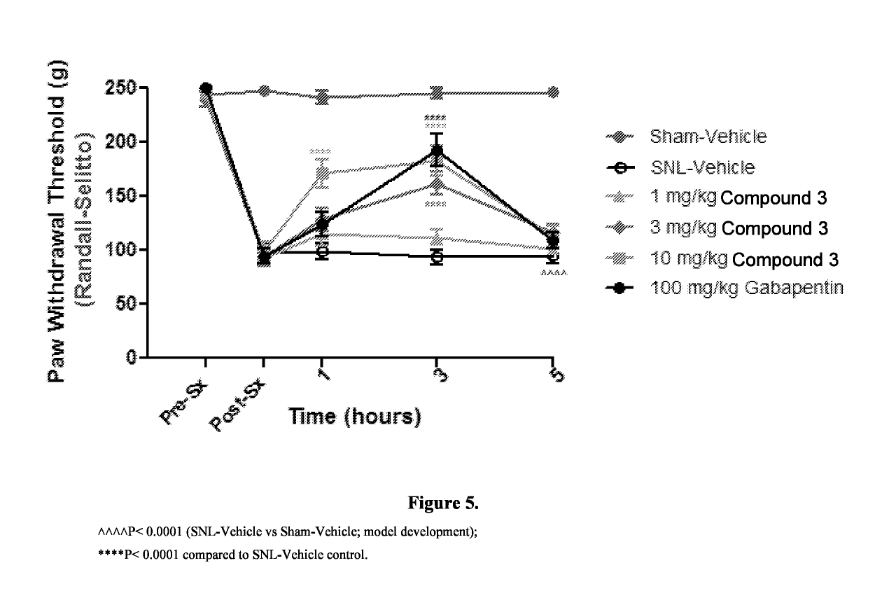

Compound 3 was tested using the procedures described in Example 1. Compound 3 reduced SNL-induced mechanical hyperalgesia in rats when dosed orally at ≥3 mg/kg one hour before testing. Compound 3 showed robust efficacy in the SNL-induced mechanical hyperalgesia model of neuropathic pain in rats, with 56% maximum reversal achieved at 3 hours following IP dosing (see FIG. 5). Efficacy was most notable at a dosing of 10 mg/kg; the MED was 3 mg/kg. In the experiment, male, Sprague-Dawley rats, 261-328 g (n=8-11/group) were baselined for left hind paw threshold before (Pre-Sx) and then 3 weeks post-SNL surgery. Compound 3 and gabapentin, the positive control, were administered IP 1 hour prior to testing. Compound 3 was formulated in 25% HPBCD while gabapentin was dissolved in 0.9% normal saline.

### Example 7. The Effect of TAAR1 Agonist Compound 2 on SNL-Induced Mechanical Hyperalgesia in Rats Following PO Dosing

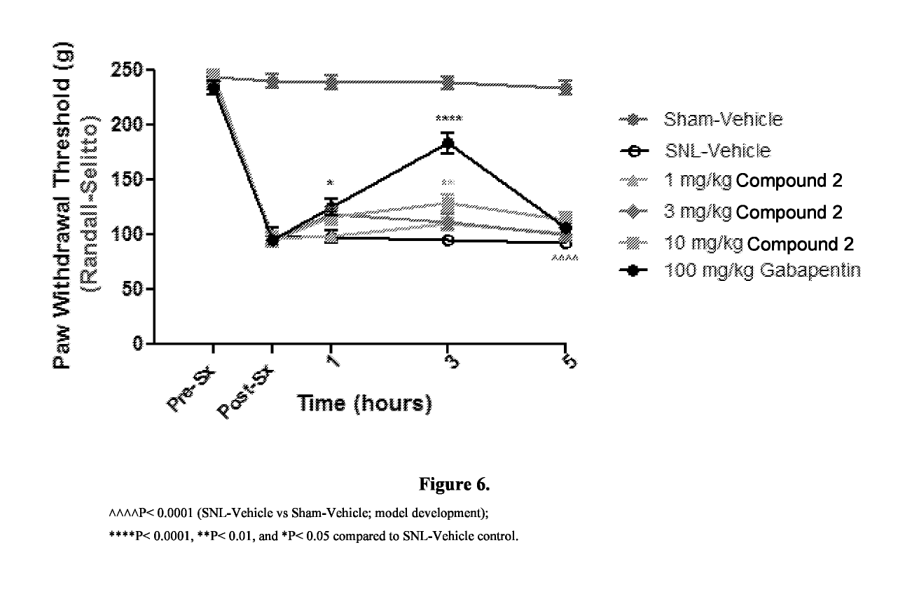

Compound 2 was tested using the procedures described in Example 1. Compound 2 reduced SNL-induced mechanical hyperalgesia in rats when dosed orally at 10 mg/kg one hour before testing. Compound 2 showed efficacy in the SNL-induced mechanical hyperalgesia model of neuropathic pain in rats, with 22% maximum reversal achieved at 3 hours following PO dosing (see FIG. 6). Efficacy was most notable at a dosing of 10 mg/kg; the MED was 10 mg/kg. In the experiment, male, Sprague-Dawley rats, 266-334 g (n=8-11/group) were baselined for left hind paw threshold before (Pre-Sx) and then 3 weeks post-SNL surgery. Compound 2 (PO) and gabapentin (IP), the positive control, were administered 1 hour prior to testing. Compound 2 was formulated in 0.5% methyl-cellulose while gabapentin was dissolved in 0.9% normal saline.

### Example 8. The Effect of TAAR1 Agonist Compound 2 on SNL-Induced Mechanical Hyperalgesia in 254 bp β-Arrestin₂ KO Rats Following IP Dosing

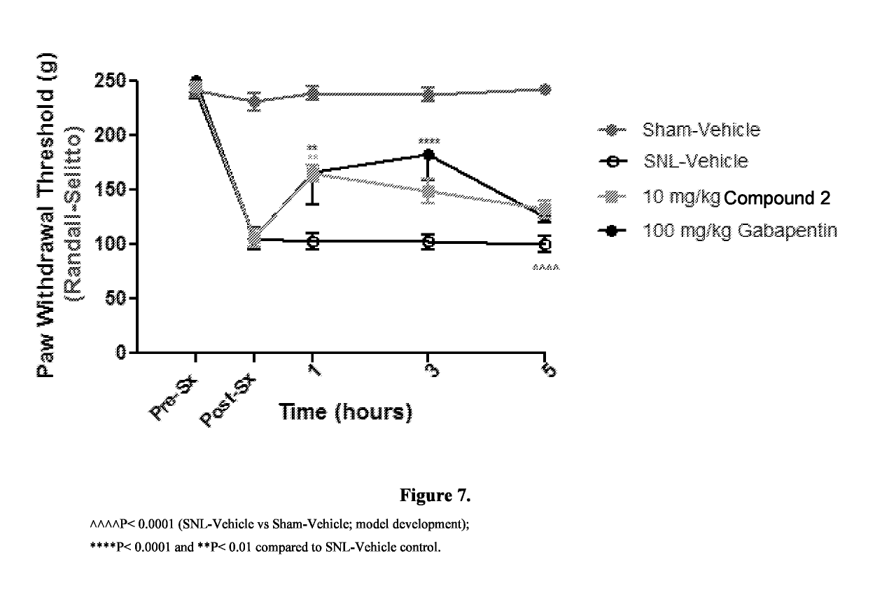

Compound 2 was tested using the procedures described in Example 1. Compound 2 reduced SNL-induced mechanical hyperalgesia in rats when dosed IP one hour before testing. Compound 2 showed robust efficacy in the SNL-induced mechanical hyperalgesia model of neuropathic pain in rats, with 43% maximum reversal achieved at 1 hour following IP dosing (see FIG. 7). In the experiment, male, 254 bp β-arrestin₂ KO rats, 282-388 g (n=4-11/group) were baselined for left hind paw threshold before (Pre-Sx) and then 4 weeks post-SNL surgery. Compound 2 (IP) and gabapentin (subcutaneous, SC), the positive control, were administered 1 hour prior to testing. Compound 2 was formulated in 25% hydroxypropyl-β-cyclodextrin while gabapentin was dissolved in 0.9% normal saline.

### Example 9. The Effect of TAAR1 Agonist Compound 2 on SNL-Induced Mechanical Hyperalgesia in 1276 bp GRK5 KO Rats Following IP Dosing

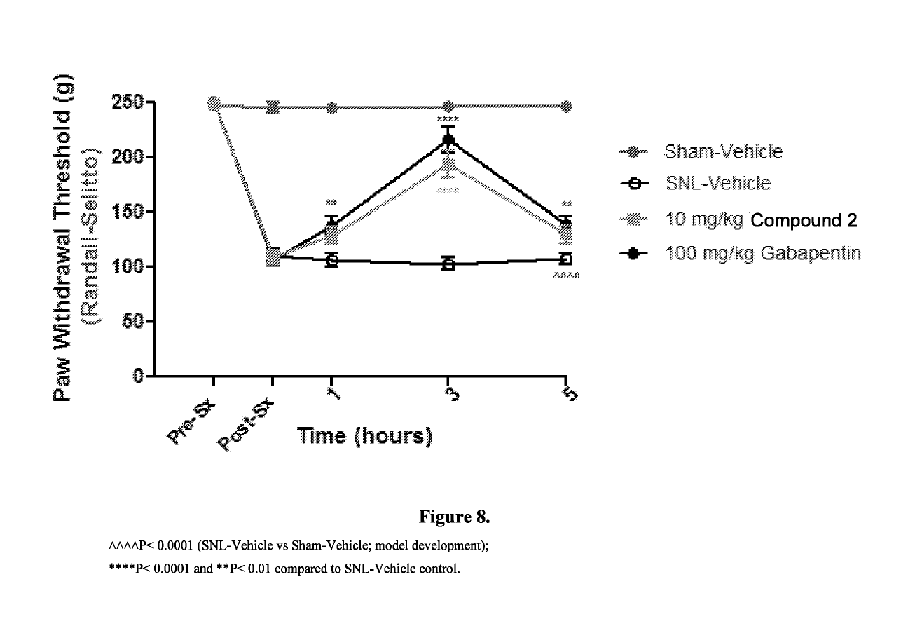

Compound 2 was tested using the procedures described in Example 1. Compound 2 reduced SNL-induced mechanical hyperalgesia in rats when dosed IP one hour before testing. Compound 2 showed robust efficacy in the SNL-induced mechanical hyperalgesia model of neuropathic pain in rats, with 61% maximum reversal achieved at 3 hours following IP dosing (see FIG. 8). In the experiment, male, 1276 bp GRK5 KO rats, 305-410 g (n=8-11/group) were baselined for left hind paw threshold before (Pre-Sx) and then 3 weeks post-SNL surgery. Compound 2 (IP) and gabapentin (IP), the positive control, were administered 1 hour prior to testing. Compound 2 was formulated in 25% hydroxypropyl-β-cyclodextrin while gabapentin was dissolved in 0.9% normal saline.

### Example 10. Rotarod Performance

The Rotarod test is used to evaluate motor coordination, strength, and balance. The animals are placed on the rotating rod of the device. The time during which an animal can maintain itself on said bar at an accelerating speed of 5-40 rpm is recorded. 300 seconds is the assay cut-off. Data are analyzed by a two-way ANOVA using a Bonferroni Multiple Comparisons test.

### Example 11. The Effect of TAAR1 Partial Agonist Compound 1 on Rotarod Performance in Rats

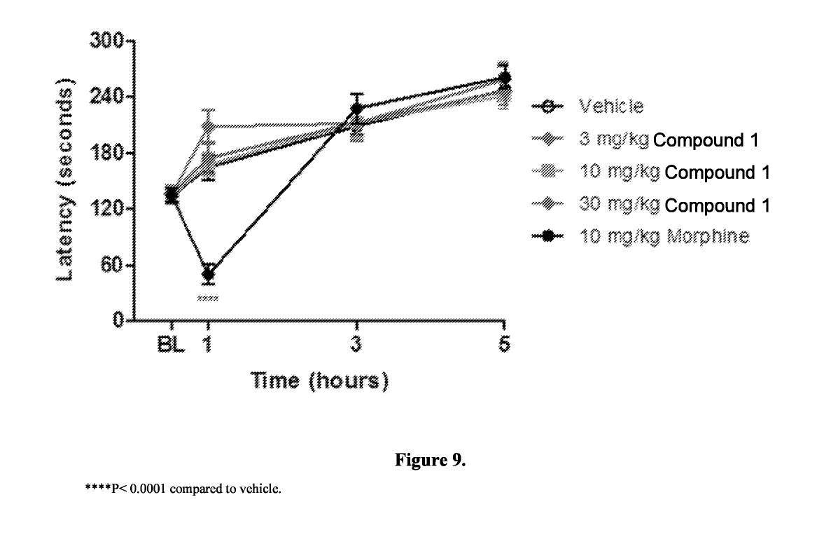

Compound 1 was tested using the procedures as described in Example 10. Compound 1 did not produce ataxia when dosed orally at 3 mg/kg, 10 mg/kg, and 30 mg/kg (see FIG. 9). In the experiment, male, Sprague-Dawley rats, 211-241 g (n=10/group) were tested on an accelerating rotarod (AccuScan; manual 5-40 rpm). Latency to fall off was assessed over 5 minutes the day prior to the experiment (avg. baseline (BL)×2) and then 1, 3, and 5 hours post-compound administration. Compound 1 (PO) was formulated in 0.5% methyl-cellulose, while morphine sulfate (SC), the positive control, was dissolved in 0.9% NaCl.

### Example 12. The Effect of TAAR1 Agonist Compound 2 on Rotarod Performance in Rats

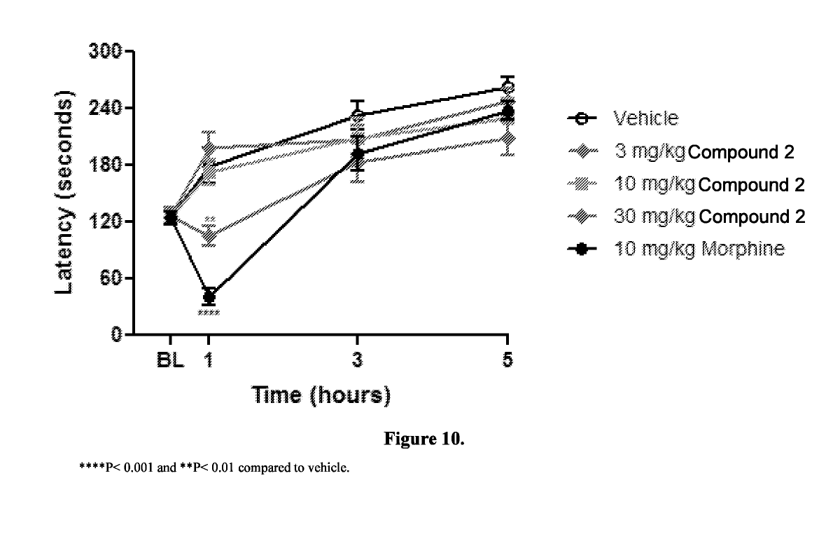

Compound 2 was tested using the procedures as described in Example 10. Compound 2 produced deficits in motor performance 1 hour post dosing when dosed IP at 30 mg/kg (see FIG. 10). In the experiment, male, Sprague-Dawley rats, 216-238 g (n=10/group) were tested on an accelerating rotarod (AccuScan; manual 5-40 rpm). Latency to fall off was assessed over 5 minutes the day prior to the experiment (avg. baseline (BL)×2) and then 1, 3, and 5 hours post-compound administration. Compound 2 (IP) was formulated in 25% hydroxypropyl-β-cyclodextrin, while morphine sulfate (SC), the positive control, was dissolved in 0.9% NaCl.

### Example 13. The Effect of TAAR1 Agonist Compound 2 on Nociceptive Pain or Analgesia

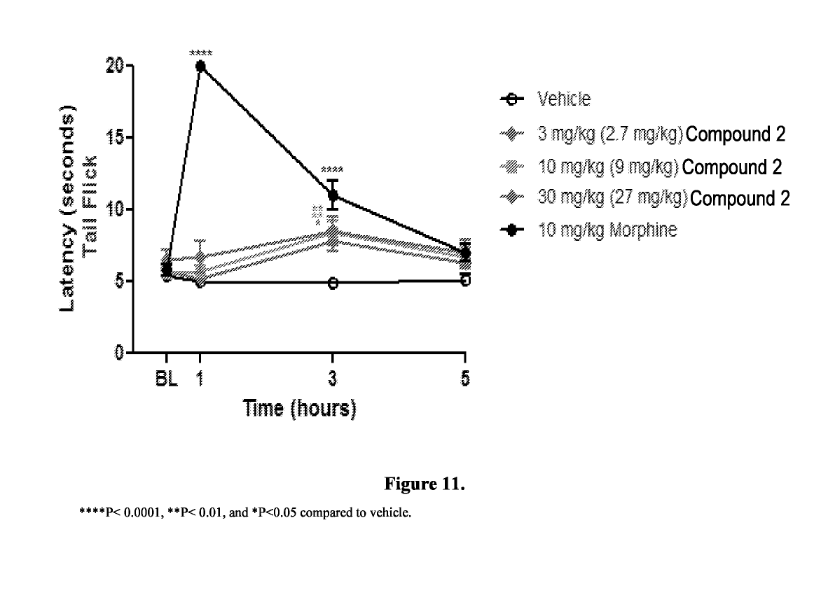

To assess the actions of a TAAR1 agonist or partial agonist of the present disclosure for the treatment or prevention of acute pain, the rat tail flick can be used. Compound 2 was tested using the procedures described above. Compound 2 only demonstrated weak to marginal analgesic efficacy in the tail flick assay at tested doses of 3 mg/kg, 10 mg/kg, and 30 mg/kg, as compared to the positive control morphine, which produced robust analgesic efficacy (see FIG. 11). In the experiment, male, Sprague-Dawley rats, 224-255 g (n=8-11/group) were baselined for tail flick latency using a radiant heat tail flick apparatus (intensity set at 40) the day prior to dosing; cutoff was set to 20 seconds. Compound 2 was formulated in 25% hydroxypropyl-β-cyclodextrin and administered IP, while morphine sulfate, the positive control, was dissolved in saline and administered subcutaneously. All compounds were administered one hour prior to testing. Doses were 10% less than represented due to salt factor.

### Example 14. The Effect of TAAR1 Agonist Compound 2 on Nociceptive Pain or Analgesia

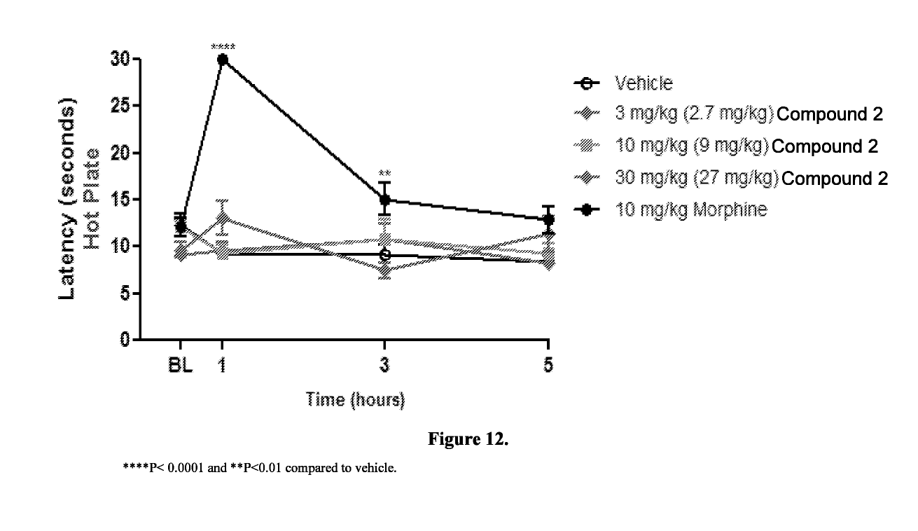

Compound 2 was tested using the hot plate test procedures as described above. Compound 2 was not efficacious as an analgesic in the hot plate assay at tested doses of 3 mg/kg, 10 mg/kg, and 30 mg/kg, as compared to the positive control morphine (see FIG. 12). In the experiment, male, Sprague-Dawley rats, 224-255 g (n=8-11/group) were baselined for paw latency on a hot plate which is set to 52° C. one day prior to dosing; cutoff was set to 30 seconds. Compound 2 was formulated in 25% hydroxypropyl-β-cyclodextrin while morphine sulfate, the positive control, was dissolved in 0.9% NaCl. Compound 2 was administered IP while morphine was administered SC one hour prior to testing. Doses were 10% less than represented due to salt factor.

The results of the Examples indicate that compounds of the present disclosure demonstrate analgesic activity. Compounds 1, 2, and 3 of the present disclosure demonstrate analgesic activity.

### Example 15. TAAR1 Biodata

Useful compounds of the disclosure were screened in a hTAAR1 functional assay. EC₅₀ and Eₘₐₓ values are reported for these compounds in Table 1.

TABLE 1
TAAR1 Biodata

| | hTAAR1 EC₅₀ | hTAAR1 Eₘₐₓ |
|---|---|---|
| Compound | (mean ± SEM(n)) | (mean ± SEM(n)) |
| 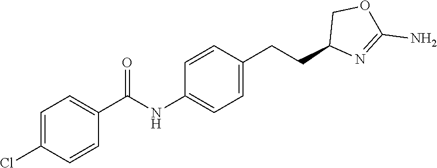 [SMILES: NC1=N[C@@H](CCc2ccc(NC(=O)c3ccc(Cl)cc3)cc2)CO1] | 8.530 ± 0.250 nM(3) | 42.7 ± 3.0% (9.03) |
| 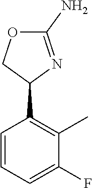 | 13.720 ± 5.010 nM(3) | 68.7 ± 3.0% (6.82) |
| 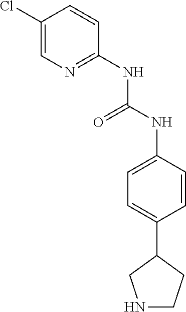 [SMILES: O=C(Nc1ccc(C2CCNC2)cc1)Nc1ccc(Cl)cn1] | 54.510 ± 18.650 nM(3) | 80.9 ± 3.0% (1.98) |
| 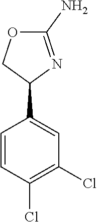 [SMILES: NC1=N[C@@H](c2ccc(Cl)c(Cl)c2)CO1] | 83.450 ± 23.870 nM(3) | 35.5 ± 3.0% (10.27) |
| 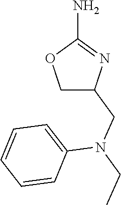 [SMILES: CCN(CC1COC(N)=N1)c1ccccc1] | 92.010 ± 10.900 nM(3) | 81.7 ± 3.0% (10.49) |
| 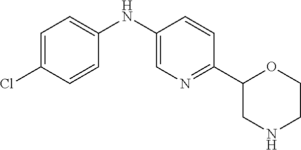 [SMILES: Clc1ccc(Nc2ccc(C3CNCCO3)nc2)cc1] | 123.350 ± 9.986 nM(3) | 56.1 ± 7.5% (3) |
| 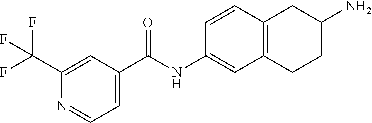 [SMILES: NC1CCc2cc(NC(=O)c3ccnc(C(F)(F)F)c3)ccc2C1] | 162.540 ± 35.490 nM(3) | 85.8 ± 3.0% (2.8) |
| 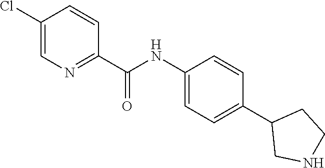 [SMILES: O=C(Nc1ccc(C2CCNC2)cc1)c1ccc(Cl)cn1] | 475.200 ± 50.100 nM(3) | 73.5 ± 3.0% (7.47) |
| 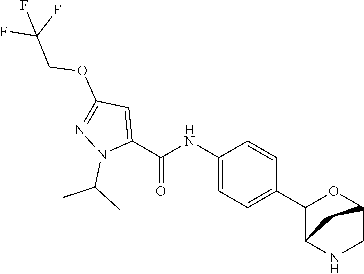 | 518.870 ± 106.585 nm(3) | 66.6 ± 14.9% (3) |
| 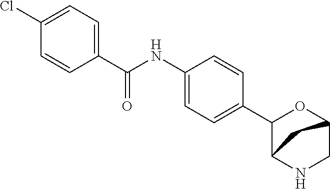 | 7209.870 ± 2790.130 nm(3) | 29.7 ± 14.2% (3) |
| 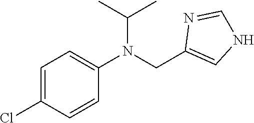 [SMILES: CC(C)N(Cc1c[nH]cn1)c1ccc(Cl)cc1] | >10 μM(3) | 10.0 ± 3.0% (3.45) |
| 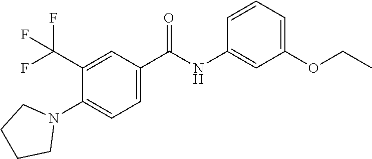 [SMILES: CCOc1cccc(NC(=O)c2ccc(N3CCCC3)c(C(F)(F)F)c2)c1] | >10 μM(3) | 6.1 ± 3.0% (1.84) |

### Example 16. GPCR cAMP Modulation Assay

A GPCR cAMP Modulation Assay can be used to determine whether a compound can be considered an agonist or antagonist.

### Cell Handling

cAMP Hunter cell lines were expanded from freezer stocks according to standard procedures. Cells were seeded into white walled, 384-well microplates in a total volume of 20 μL and incubated at 37° C. prior to testing. cAMP modulation was determined using a DiscoverX HitHunter cAMP XS+ assay.

### Gs Agonist Format

To determine whether a compound would be considered an agonist, cells were incubated with a test compound to induce a response. Media was aspirated from cells and replaced with 15 μL 2:1 HBSS/10 mM Hepes: cAMP XS+Ab reagent. Sample stocks were diluted immediately to generate a 4× sample in assay buffer. 5 μL of the 4× sample was added to cells and incubated at room temperature or 37° C. for 30 or 60 minutes. Final assay vehicle concentration was 1%.

### Gi Agonist Format

To determine whether a compound would be considered an agonist, cells were incubated with a test compound in the presence of EC₈₀ forskolin to induce a response. Media was aspirated from cells and replaced with 15 μL 2:1 HBSS/10 mM Hepes: cAMP XS+Ab reagent. Sample stocks were diluted immediately to generate a 4× sample in assay buffer containing 4× EC₈₀ forskolin. 5 μL of 4× sample was added to cells and incubated at room temperature or 37° C. for 30 or 60 minutes. Final assay vehicle concentration was 1%.

### Allosteric Modulation Format

To determine whether a compound would be considered allosteric, cells were pre-incubated with a test compound followed by agonist induction at the EC₂₀ concentration. Media was aspirated from cells and replaced with 10 μL 1:1 HBSS/10 mM Hepes: cAMP XS+Ab reagent. Sample stocks were diluted immediately to generate a 4× sample in assay buffer. 5 μL of 4× compound was added to the cells and incubated at room temperature or 37° C. for 30 minutes. 5 μL of 4× EC₂₀ agonist was added to the cells and incubated at room temperature or 37° C. for 30 or 60 minutes. For Gi-coupled GPCRs, EC₈₀ forskolin was included.

### Inverse Agonist Format (Gi Only)

To determine whether a compound would be considered an inverse agonist, cells were pre-incubated with sample in the presence of EC₂₀ forskolin. Media was aspirated from cells and replaced with 15 μL 2:1 HBSS/10 mM Hepes: cAMP XS+Ab reagent. Sample stocks were immediately diluted to generate a 4× sample in assay buffer containing 4× EC₂₀ forskolin. 5 μL of 4× sample was added to cells and incubated at room temperature or 37° C. for 30 or 60 minutes. Final assay vehicle concentration was 1%.

### Antagonist Format

To determine whether a compound would be considered an antagonist, cells were pre-incubated with a test compound followed by agonist challenge at the EC₈₀ concentration. Media was aspirated from cells and replaced with 10 μL 1:1 HBSS/Hepes: cAMP XS+Ab reagent. 5 μL of 4× compound was added to the cells and incubated at room temperature or 37° C. for 30 minutes. 5 μL of 4× EC₈₀ agonist was added to cells and incubated at room temperature or 37° C. for 30 or 60 minutes. For Gi coupled GPCRs, EC₈₀ forksolin was included.

### Signal Detection

After appropriate compound incubation, assay signal was generated by incubation with 20 μL cAMP XS+ED/CL lysis cocktail for one hour followed, which was followed with room temperature incubation with 20 μL cAMP XS+EA reagent for three hours. Microplates were read following signal generation with a PerkinElmer Envision™ instrument for chemiluminescent signal detection.

### Data Analysis

CBIS data analysis suite (ChemInnovation, CA) was used to analyze compound activity. RLU stands for relative light units.

For Gs agonist mode assays, percent activity was calculated using the following formula:

$$\% Activity = \frac{\begin{matrix}[ ( mean RLU of test sample - \\ mean RLU of vehicle control ) ]\end{matrix}}{\begin{matrix}[ ( mean RLU of MAX control - \\ mean RLU of vehicle control ) ]\end{matrix}} \times 100$$

For Gs positive allosteric mode assays, percentage modulation is calculated using the following formula:

$$\% Modulation = \frac{\begin{matrix}[ ( mean RLU of test sample - \\ mean RLU of {EC}_{20} control ) ]\end{matrix}}{\begin{matrix}[ ( mean RLU of MAX control - \\ mean RLU of {EC}_{20} control ) ]\end{matrix}} \times 100$$

For Gs antagonist or negative allosteric mode assays, percentage inhibition is calculated using the following formula:

$$\% Inhibition = \frac{\begin{matrix}[ ( 1 - ( mean RLU of test sample - \\ mean RLU of vehicle control ) ) ]\end{matrix}}{\begin{matrix}[ ( mean RLU of {EC}_{80} control - \\ mean RLU of vehicle control ) ]\end{matrix}} \times 100$$

For Gi agonist mode assays, percentage activity is calculated using the following formula:

$$\% Activity = \frac{\begin{matrix}[ ( 1 - ( mean RLU of test sample - \\ mean RLU of MAX control ) ) ]\end{matrix}}{\begin{matrix}[ ( mean RLU of vehicle control - \\ mean RLU of MAX control ) ]\end{matrix}} \times 100$$

For Gi positive allosteric mode assays, percentage modulation is calculated using the following formula:

$$\% Modulation = \frac{\begin{matrix}[ ( 1 - ( mean RLU of test sample - \\ mean RLU of MAX control ) ]\end{matrix}}{\begin{matrix}[ ( mean RLU of {EC}_{20} control - \\ mean RLU of MAX control ) ]\end{matrix}} \times 100$$

For Gi inverse agonist mode assays, percentage activity is calculated using the following formula:

$$\begin{matrix}\% Inverse Agonist \\ Activity\end{matrix} = \frac{\begin{matrix}[ ( mean RLU of test sample - \\ mean RLU of {EC}_{20} forskolin ) ]\end{matrix}}{\begin{matrix}[ ( mean RLU of forskolin positive control - \\ mean RLU of {EC}_{20} control ) ]\end{matrix}} \times 100$$

For Gi antagonist or negative allosteric mode assays, percentage inhibition is calculated using the following formula:

$$\% Inhibition = \frac{\begin{matrix}[ ( mean RLU of test sample - \\ mean RLU of {EC}_{80} control ) ]\end{matrix}}{\begin{matrix}[ ( mean RLU of forskolin positive control - \\ mean RLU of {EC}_{80} control ) ]\end{matrix}} \times 100$$

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

Having now fully described this invention, it will be understood by those of ordinary skill in the art that the same can be performed within a wide and equivalent range of conditions, formulations and other parameters without affecting the scope of the invention or any embodiment thereof.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

All patents, patent applications, and other non-patent publications cited herein are fully incorporated by reference herein in their entirety.

1. A method of treating pain in a subject, comprising administering to a subject in need of such treating a therapeutically effective amount of a compound that is a TAAR1 agonist or partial agonist.

2. The method according to claim 1, wherein said compound is a compound of Formula (I): 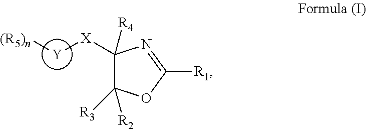
or a pharmaceutically acceptable salt, a racemic mixture, an enantiomer and/or optical isomer (stereoisomer), a tautomer, a prodrug, or a solvate thereof,
wherein:
R₁ is NH₂, NH(lower alkyl), or 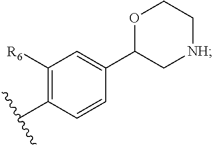
R₂ and R₃ are independently hydrogen or lower alkyl optionally substituted by halogen;
R₄ is hydrogen, phenyl, or lower alkyl;
R₅, each independently, is hydrogen, deuterium, tritium, cyano, halogen, lower alkyl optionally substituted by halogen, lower alkoxy optionally substituted by halogen, phenyl optionally substituted by halogen, phenyloxy, benzyl, benzyloxy,
—C(O)O-lower alkyl, —NHC(O)-aryl wherein aryl is optionally substituted by lower alkyl or halogen, —O—(CH₂)ₒ—O-lower alkyl, —NH-cycloalkyl, cycloalkyl, piperidin-1-yl, or tetrahydropyran-4-yloxy, wherein the optional substituents for each R₅, are the same or different;
R₆ is hydrogen or halogen;
X is a bond, —(CHR)ₘ, —O(CHR)ₘ, —NRCHR′—,
CHROCHR′—, —SCHR—, —S(O)₂CH₂—, —CH₂SCH₂—,
—CH₂N(R)CHR′—, -cycloalkyl-(CHR)ₘ, or —SiRR′—CH₂—;
R and R′ are each independently hydrogen, lower alkyl optionally substituted by halogen, or benzyl optionally substituted by alkoxy or halogen, and when m>1, each R is the same or different;
Y is aryl, heteroaryl, cycloalkyl, or heterocycloalkyl;
m is 1, 2, 3, or 4;
n is 0, 1, 2, or 3; and
o is 2 or 3.

3. The method according to claim 2, wherein in the compound of Formula (I), Y is phenyl, naphthyl, thiophenyl, pyridinyl, cyclohexyl, 1,2,3,4-tetrahydro-naphthalen-2-yl, 2,3-dihydrobenzo[1,4]dioxin-6-yl, benzo[1,3]dioxol-5-yl, pyrimidyl, indanyl, 2,3-dihydroindol-1-yl, or 3,4-dihydro-quinolin-1-yl.

4-6. (canceled)

7. The method according to claim 2, wherein in the compound of Formula (I),
R₁ is NH₂;
R₂ and R₃ are hydrogen or C₁-C₇ alkyl;
R₄ is hydrogen or C₁-C₇ alkyl;
R₅ is hydrogen or halogen;
X is a bond, —N(C₂H₅)CH₂—, or —CH(C₂H₅)CH₂—; and
Y is phenyl.

8. (canceled)

9. The method according to claim 2, wherein the compound is of the structure: 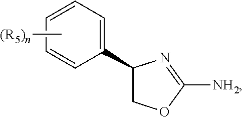
or a pharmaceutically acceptable salt, a racemic mixture, an enantiomer and/or optical isomer (stereoisomer), a tautomer, a prodrug, or a solvate thereof.
wherein
n is 1 or 2; and
R₅, each independently, is halogen or lower alkyl optionally substituted by halogen.

10. (canceled)

11. The method according to claim 9, wherein the compound is: 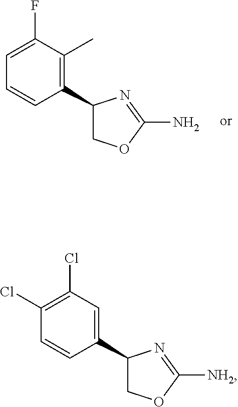
or a pharmaceutically acceptable salt, a racemic mixture, an enantiomer and/or optical isomer (stereoisomer), a tautomer, a prodrug, or a solvate thereof.

12. The method according to claim 2, wherein the compound is of the structure: 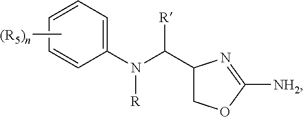
or a pharmaceutically acceptable salt, a racemic mixture, an enantiomer and/or optical isomer (stereoisomer), a tautomer, a prodrug, or a solvate thereof.
wherein R and R′ are each independently hydrogen or lower alkyl optionally substituted by halogen.

13. (canceled)

14. The method according to claim 12, wherein the compound is of the structure: 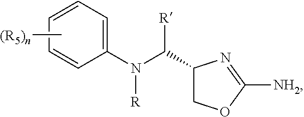
or a pharmaceutically acceptable salt, a racemic mixture, an enantiomer and/or optical isomer (stereoisomer), a tautomer, a prodrug, or a solvate thereof,
wherein R and R′ are each independently hydrogen or lower alkyl optionally substituted by halogen.

15. The method according to claim 14, wherein the compound is of the structure: 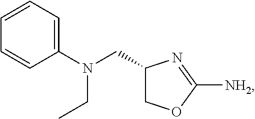 [SMILES: CCN(C[C@H]1COC(N)=N1)c1ccccc1]
or a pharmaceutically acceptable salt, a racemic mixture, an enantiomer and/or optical isomer (stereoisomer), a tautomer, a prodrug, or a solvate thereof.

16. The method according to claim 2, wherein the compound is of the structure: 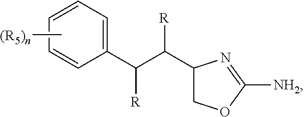
or a pharmaceutically acceptable salt, a racemic mixture, an enantiomer and/or optical isomer (stereoisomer), a tautomer, a prodrug, or a solvate thereof,
wherein each R is the same or different.

17. The method according to claim 16, wherein the compound is of the structure: 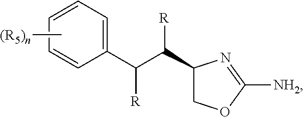
or a pharmaceutically acceptable salt, a racemic mixture, an enantiomer and/or optical isomer (stereoisomer), a tautomer, a prodrug, or a solvate thereof,
wherein each R is the same or different.

18. (canceled)

19. The method according to claim 16, wherein the compound is of the structure: 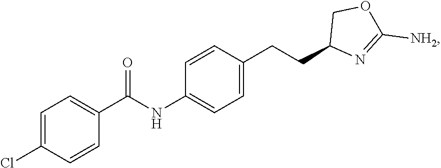 [SMILES: NC1=N[C@@H](CCc2ccc(NC(=O)c3ccc(Cl)cc3)cc2)CO1]
or a pharmaceutically acceptable salt, a racemic mixture, an enantiomer and/or optical isomer (stereoisomer), a tautomer, a prodrug, or a solvate thereof.

20. The method according to claim 1, wherein said compound is a compound of Formula (IIa) or (IIb): 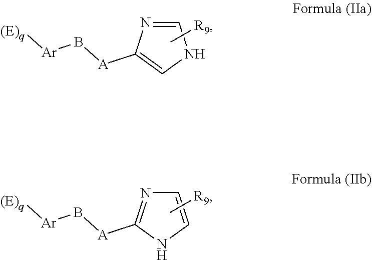
or a pharmaceutically acceptable salt, a racemic mixture, an enantiomer and/or optical isomer (stereoisomer), a tautomer, a prodrug, or a solvate thereof,
wherein:
—A—B— is —CH(R₈)—, —N(R₇)—CH(R₈)—, —CH(R₈)—N(R₇)—, —NH—NH—, —O—CH(R₈)—, —CH(R₈)—O—, —S—CH(R₈)—, —CH(R₈)—S—, or —CH(R₈)—CH(R₈)—;
R₇ and R₈ are each independently hydrogen, lower alkyl, lower alkenyl, cycloalkyl, lower alkyl substituted by hydroxy or halogen, —(CH₂)_{d}—S-lower alkyl, —(CH₂)_{d}—O-lower alkyl, —(CH₂)_{d}—NHC(O)O-lower alkyl, —(CH₂)_{d}-aryl, or —(CH₂)_{d}-heteroaryl;
R₉ is hydrogen, halogen, lower alkyl, or amino;
Ar is phenyl, naphthyl, benzofuranyl, benzo[1,3]dioxolyl, pyrimidin-2-yl, pyrimidin-4-yl, or pyridin-3-yl;
E is hydrogen, halogen, lower alkyl optionally substituted by one or more halogens, lower alkoxy optionally substituted by one or more halogens,
—(CH₂)ₚ-aryl, —(CH₂)ₚ-heteroaryl, —O—(CH₂)ₚ-aryl, —O—(CH₂)ₚ-heteroaryl, —(CH₂)ᵣ-phenyl optionally substituted by lower alkoxy, —(CH₂)ᵣ C(O)-phenyl optionally substituted by lower alkoxy, —(CH₂)ᵣ—O-phenyl optionally substituted by lower alkoxy, cycloalkyl, morpholinyl, NO₂, amino, hydroxy, —CH(OH)-phenyl, or —NHC(O)aryl;
p is 0 or 1;
q is 0, 1, 2, 3, or 4;
r is 0, 1, 2, 3; and
d is 0, 1, 2, or 3.

21-24. (canceled)

25. The method according to claim 20, wherein the compound is of the structure: 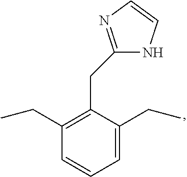 [SMILES: CCc1cccc(CC)c1Cc1ncc[nH]1]
or a pharmaceutically acceptable salt, a racemic mixture, an enantiomer and/or optical isomer (stereoisomer), a tautomer, a prodrug, or a solvate thereof.

26. The method according to claim 1, wherein said compound is a compound of Formula (III): 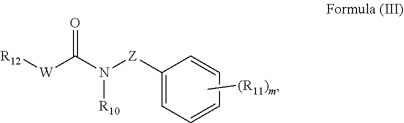
or a pharmaceutically acceptable salt, a racemic mixture, an enantiomer and/or optical isomer (stereoisomer), a tautomer, a prodrug, or a solvate thereof,
wherein:
R₁₀ is hydrogen or lower alkyl;
m is 1 or 2;
R₁₁ independently is —(CH₂)ₖ—(O)ⱼheterocycloalkyl, or —C(O)-heterocycloalkyl,
wherein the heterocycloalkyl group is optionally substituted by lower alkyl, hydroxy, halogen, or —(CH₂)_{b}-aryl; or two R₁₁, together with the ring atoms they are attached to, forms heterocycloalkyl optionally substituted by amino,
k is 0, 1 or 2;
j is 0 or 1;
b is 0, 1 or 2;
R₁₂ is (i) lower alkyl, optionally substituted by one or more (same or different) halogens, or cycloalkyl optionally substituted by lower alkoxy or halogen; orindan-2-yl; or
(ii) heterocycloalkyl, optionally substituted by heteroaryl; or
(iii) aryl or heteroaryl, wherein the aromatic rings in aryl and heteroaryl are optionally substituted by one or two substituents independently selected from the group of lower alkyl, halogen, heteroaryl, hydroxy, CF₃, OCF₃, OCH₂CF₃, OCH₂-cycloalkyl, OCH₂C(CH₂OH)(CH₂Cl)(CH₃), S-lower alkyl, lower alkoxy, CH₂-lower alkoxy, lower alkynyl, cyano, —C(O)-phenyl, —O-phenyl, —O—CH₂-phenyl, phenyl, and —CH₂-phenyl, and wherein the phenyl rings are optionally substituted by halogen, —C(O)-lower alkyl, —C(O)OH, or
—C(O)O-lower alkyl, or the aromatic rings are optionally substituted by heterocycloalkyl, OCH₂-oxetan-3-yl, or O-tetrahydropyran-4-yl, optionally substituted by lower alkyl;
W is a bond, —N(R₁₃)—, —CH₂NH—, —CH(R₁₄)—, —(CHR₁₄)ᵥ—O—, —O—(CHR₁₄)ᵥ, or —(CH₂)₂—;
Z is a bond or —CH₂—,
R₁₃ is hydrogen or lower alkyl;
R₁₄ is hydrogen, lower alkyl, optionally substituted by one or more (same or different) halogens, or lower alkoxy; and
v is 0, 1,2 or 3.

27. The method according to claim 26, wherein said compound is a compound of Formula (IIIa), 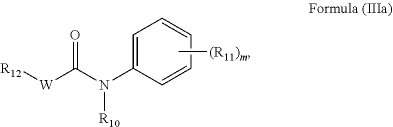
or a pharmaceutically acceptable salt, a racemic mixture, an enantiomer and/or optical isomer (stereoisomer), a tautomer, a prodrug, or a solvate thereof,
wherein:
R₁₀ is hydrogen or lower alkyl;
m is 1 or 2;
R₁₁ each independently is —(CH₂)ₖ—(O)ⱼ-heterocycloalkyl optionally substituted by lower alkyl, hydroxy, halogen, or —(CH₂)_{b}-aryl; or two R₁₁, together with the ring atoms they are attached to, forms heterocycloalkyl optionally substituted by amino,
k is 0, 1 or 2;
j is 0 or 1;
b is 0, 1 or 2;
R₁₂ is cycloalkyl, heterocycloalkyl, aryl, or heteroaryl, wherein the aromatic rings within the aryl or heteroaryl group are optionally substituted by one or two substituents independently selected from the group of lower alkyl, halogen, heteroaryl, CF₃, —OCF₃, —OCH₂CF₃, lower alkoxy, —CH₂-lower alkoxy, lower alkynyl, and cyano;
W is a bond, —N(R₁₃)—, —CH₂NH—, —CH(R₁₄)—, —(CH₂)ᵥ,—O—, or —(CH₂)₂—;
R₁₃ is hydrogen or lower alkyl;
R₁₄ is hydrogen, lower alkyl, or lower alkoxy; and
v is 0, 1, or 2.

28-29. (canceled)

30. The method according to claim 26, wherein the compound is of the structure: 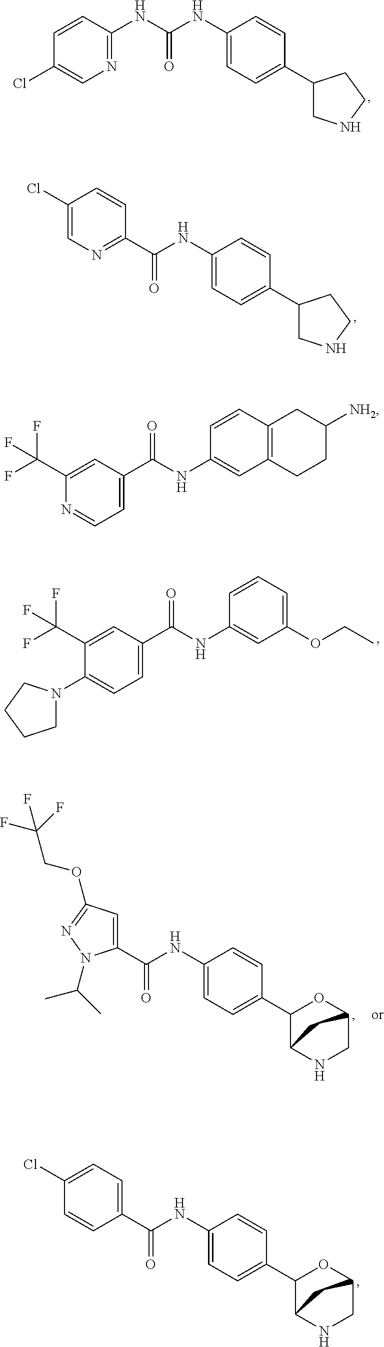
or a pharmaceutically acceptable salt, a racemic mixture, an enantiomer and/or optical isomer (stereoisomer), a tautomer, a prodrug, or a solvate thereof.

31. The method according to claim 1, wherein said compound is a compound of Formula (IV): 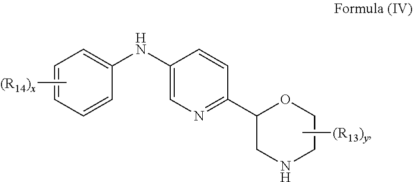
or a pharmaceutically acceptable salt, a racemic mixture, an enantiomer and/or optical isomer (stereoisomer), a tautomer, a prodrug, or a solvate thereof,
wherein:
R₁₃ is independently hydrogen, halogen, alkyl, alkoxy, haloalkyl, haloalkoxy, hydroxyl, cyano, amino, cycloalkyl, heterocycloalkyl, aryl, or heteroaryl;
R₁₄ is independently hydrogen, halogen, alkyl, alkoxy, haloalkyl, haloalkoxy, hydroxyl, cyano, amino, cycloalkyl, heterocycloalkyl, aryl, or heteroaryl;
x is 0, 1, or 2; and
y is 0, 1, or 2.

32-35. (canceled)

36. The method according to claim 31, wherein the compound is of the structure: 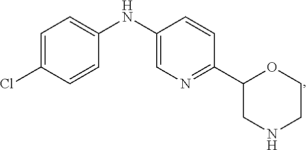 [SMILES: Clc1ccc(Nc2ccc(C3CNCCO3)nc2)cc1]
or a pharmaceutically acceptable salt, a racemic mixture, an enantiomer and/or optical isomer (stereoisomer), a tautomer, a prodrug, or a solvate thereof.

37-43. (canceled)

44. The method according to claim 1, wherein the method is for treating one or more of acute, chronic, or neuropathic pain.

45-47. (canceled)
